SEQUENCE LISTING (20 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 537 bp, Homo sapiens
  source 1..537: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 3 - protein, 280 aa, synthetic construct
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNRGIL

SEQ ID NO: 4 - DNA, 843 bp, synthetic construct
  source 1..843: mol_type = other DNA
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagggggatcctctag

SEQ ID NO: 5 - DNA, 8475 bp, synthetic construct
  source 1..8475: mol_type = other DNA
tggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcttaggttggagtgatacatttttatcacttttacccgtctttggattaggcagtagctctgacggccctcctgtcttaggttagtgaaaaatgtcactctcttacccgtcattggctgtccagcttagctcgcaggggaggtggtctggatccaccatgtctagaggatccccctgttccacctcttgaaggctatgtaggccacaaggcccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcggggcactcctcgcacacggtgttctgcttgtcctggcaggagaacacgaggcccgagcccgcctcgcacacgcggcacgcctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatggcgcggccggtggcacctgcccccatcgcccgcctcccgcggcagcgctcgacttccagctcggtccgctttgcggactgatggggctgcgctgcgctgcgctccagcgccccccctgcccgccggagctggccgcggcccgaattcctgcaggaattcgatggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccccggtctgcaggaattcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagtcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaagcggccgcacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaacattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgactatgtctttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttgtatgcttccggctcgtatgttgtgtggaattgtgagcggataacaatttcacacaggaaacagctatgaccatgattacgccaagccgaattaaccctcactaaagggaacagctagc

SEQ ID NO: 6 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 7 - DNA, 513 bp, synthetic construct
  source 1..513: mol_type = other DNA
atggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtga

SEQ ID NO: 8 - protein, 178 aa, synthetic construct
MHSSALLCCLVLLTGVRASPGQGTQSENSCTHFPGNLPNMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKIRN

SEQ ID NO: 9 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDAKAHVNSLGENLKTLRLRLRRCHRFLPCENKSKAVEQVKNAFNKLQEKGIYKAMSEFDIFINYIEAYMTMKIRN

SEQ ID NO: 10 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 11 - protein, 178 aa, Rattus norvegicus
MPGSALLCCLLLLAGVKTSKGHSIRGDNNCTHFPVSQTHMLRELRAAFSQVKTFFQKKDQLDNILLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 12 - protein, 178 aa, Macaca mulatta
MHSSALLCCLVLLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 13 - protein, 178 aa, Gorilla gorilla
MHSSALLCCLVLLTGVRASPGHGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGVYKAMSEFDIFINYIEAYMTMKIRN

SEQ ID NO: 14 - protein, 178 aa, Macaca fascicularis; identical to SEQ ID NO: 12

SEQ ID NO: 15 - protein, 178 aa, Papio anubis
MHSSALLCCLVVLTGVRASPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 16 - protein, 178 aa, Pan paniscus
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKVHVNSLGENLKTLRLRLRRCHRFLPCENKSKAVEQVKNAFNKLQEKGIYKAMSEFDIFINYIEAYMTMKIRN

SEQ ID NO: 17 - protein, 178 aa, Pan troglodytes
MHSSALLCCLVLLTGVRASPGQGTQSENSCTHFPGNLPNMLRDLRDAFSRVKTFFQMKDQLDNLLLKESLLEDFKGYLGCQALXEMIQFYLEEVMPQAENQDPDIKVHVNSLGENLKTLRLRLRRCHRFLPCENKSKAVEQVKNAFNKLQEKGIVKAMSEFDIFINYIEAYMTMKIRN

SEQ ID NO: 18 - protein, 170 aa, Synthetic construct; identical to SEQ ID NO: 6

SEQ ID NO: 19 - protein, 177 aa, synthetic construct
MHSSALLCCLVLLTGRASPGQGTQSENSCTHFPGNIPNMLRDIRDAFSRVKTEFQTKDEVDNLLLKESLLEDEKGYLGCQALSEMIQFYLEEVMPQAENQDPEAKDHVNSLGENLKTLRLRIRRCHRFLPCENKSKAVEQIKNAFNKLQEKGIYKAMSEFDIFINYIEAYMTMKIRN

SEQ ID NO: 20 - protein, 177 aa, synthetic construct
MHSSALLCCLVLLTGRASPGQGTQSENSCTHFPGNIPNMLRDIRDAFSRVKTEFQMKDQLDNLLLKESLLEDEKGYLGCQALSEMIQFYLEEVMPQAENQDPDAKAHVNSLGENLKTLRLRIRRCHRFLPCENKSKAVEQVKNAFNKLQEKGIYKAMSEFDIFINYIEAYMTMKIRN

(19) United States
(12) Patent Application Publication
(10) Pub. No.: US 2025/0177525 A1
(43) Pub. Date: Jun. 5, 2025

(54) POLYDONOR CD4+ T CELLS EXPRESSING IL-10 AND USES THEREOF

(71) Applicant: TR1X, INC., La Jolla, CA (US)

(72) Inventors: Jan Egbert DE VRIES, La Jolla, CA (US); Maria Grazia RONCAROLO, La Jolla, CA (US); Xavier PALIARD, La Jolla, CA (US); David DE VRIES, La Jolla, CA (US)

(21) Appl. No.: 18/725,659

(22) PCT Filed: Dec. 27, 2022
(86) PCT No.: PCT/US2022/082422

§ 371 Date: Jun. 28, 2024

Related U.S. Application Data
(60) Provisional application No. 63/295,389, filed on Dec. 30, 2021.

(51) Int. Cl. A61K 40/11; A61K 40/22; A61K 40/41; A61P 35/00; C07K 14/54; C07K 14/705; C12N 5/0783

(52) U.S. Cl. CPC A61K 40/11; A61K 40/22; A61K 40/418; A61P 35/00; C07K 14/5428; C07K 14/70578; C12N 5/0636; A61K 2239/48; C12N 2501/2302; C12N 2501/51; C12N 2501/515

(57) ABSTRACT

The present disclosure provides a population of polydonor CD4^{IL-10} cells generated by genetically modifying CD4⁺ T cells from at least two different T cell donors. Further provided are methods of generating the polydonor CD4^{IL-10} cells and methods of using the polydonor CD4^{IL-10} cells for immune tolerization, treating GvHD, cell and organ transplantation, cancer, autoimmune and inflammatory diseases and other immune disorders.

### 1. CROSS-REFERENCE TO RELATED APPLICATIONS

This application claims the benefit of U.S. Provisional Application No. 63/295,389, filed Dec. 30, 2021, which is hereby incorporated in its entirety by reference.

### 2. SEQUENCE LISTING

The instant application contains a Sequence Listing, which has been submitted XML format and is hereby incorporated herein by reference in its entirety. Said XML copy, created on Dec. 23, 2022, is named 37104-49835Sequence-Listing.xml, and is 30.7 kilobytes (KB) in size.

### 3. BACKGROUND

Regulatory T cells belong to a small but important subset of T cells which maintain immunological tolerance to self and non-pathogenic antigens and maintain immune homeostasis. There are two major populations of regulatory T cells—CD4⁺, FOXP3⁺CD25⁺ T cells (FOXP3⁺ cells) and type 1 regulatory T (Tr1) cells. Both FOXP3⁺ and Tr1 cells downregulate pathogenic T-cell responses in various preclinical models for organ and pancreatic islet transplantation, graft-versus-host disease (GvHD) and various autoimmune and inflammatory diseases.

Tr1 cells have been shown to be effective in clinical studies. Administration of cloned, antigen-specific, autologous Tr1 cells to patients with ongoing moderate to severe Crohn's disease resulted in objective, transient remissions (Desreumaux et al., Gastroenterology. 2012; 143(5):1207-1217.e2.). In addition, adoptive transfer of donor-derived allo-specific CD4⁺ T cell populations enriched for Tr1 cells to leukemia patients following allogeneic hematopoietic stem cell transplantation (allo-HSCT) resulted in a rapid reconstitution of the immune system and protection against microbial and viral infections, without severe GvHD. In the responder patients, long term remissions and tolerance (>7 years) resulting in cures were achieved (Bacchetta et al., Front Immunol. 2014; 5:16).

Despite these encouraging results, the production of donor-derived or autologous Tr1 cells for large scale therapy for patients with high unmet medical needs is not always feasible, is very cumbersome, and also does not allow for the generation of large quantities of pure Tr1 cells.

Recently, Locafaro and colleagues circumvented some of these problems by transducing purified CD4⁺ T cells from a single donor with a bidirectional lentiviral vector containing a human IL-10 gene. The resulting single-donor CD4^{IL-10} populations shared the major functions of naturally occurring Tr1 cells. Like Tr1 cells, single-donor CD4^{IL-10} cells produce high levels of IL-10 and downregulate the proliferation of both allogeneic CD4⁺ T cells and allogeneic CD8⁺ T cells. In addition, they are cytotoxic for both normal myeloid cells (including antigen presenting cells, APC) and myeloid leukemia cells. In a humanized xeno-GvHD model, these single-donor CD4^{IL-10} cells were shown to be effective in reducing GvHD in a humanized xeno-GvHD model while retaining graft-versus-leukemia (GvL) activity. See Locafaro et al. Mol Ther. 2017; 25(10):2254-2269 and WO 2016/146,542.

Although it is possible to produce highly purified single-donor CD4^{IL-10} cells for therapeutic use, there are still significant limitations, because of qualitative and quantitative differences between the various individual batches, which most likely are related to intrinsic differences between the various donors in addition to variations in the quality of buffy coats.

### 4. SUMMARY

The present disclosure provides a new Tr1-based therapy using a population of polydonor CD4^{IL-10} cells. Polydonor CD4^{IL-10} cells refer to CD4⁺ T cells obtained from at least two different T cell donors and then genetically modified to comprise an exogenous polynucleotide encoding IL-10. The T cell donors are third party donors who are neither a host to be treated with the polydonor CD4^{IL-10} cells nor an HSC or organ transplant donor. The polydonor CD4^{IL-10} cells are not alloantigen-specific, i.e., they have not been primed or stimulated with cells from the host before administration.

Applicant demonstrated that the polydonor CD4^{IL-10} cells have cytokine production profiles, immune suppressive- and cytotoxic capabilities comparable to those of single-donor CD4^{IL-10} cells. In addition, in vivo, they are more effective in preventing xeno GvHD mediated by CD4+ T cells than single-donor CD4^{IL-10} cells, while they do not induce GvHD by themselves. Overall, the functional properties of these polydonor CD4^{IL-10} cells both in vitro and in vivo were comparable to or better than those of single donor CD4^{IL-10} cells.

Based on these results, Applicant claims that polydonor allogeneic CD4^{IL-10} cells can be used for therapeutic purposes in GvHD, cell and organ transplantation, autoimmune- and inflammatory diseases.

Further, by using third party T cells and eliminating the requirement of allo-specificity, polydonor CD4^{IL-10} cells makes the Tr1-based cell therapy available to a larger population of patients with various genetic backgrounds.

Accordingly, in a first aspect, the present disclosure provides a population of CD4⁺ T cells that have been genetically modified to comprise an exogenous polynucleotide encoding IL-10, wherein the CD4⁺ T cells were obtained from at least two different T cell donors (polydonor CD4^{IL-10} cells).

In some embodiments, the CD4⁺ T cells were obtained from two, three, four, five, six, seven, eight, nine, or ten different T cell donors. In some embodiments, the CD4⁺ T cells in the population collectively have six, seven, eight, nine, ten, eleven, twelve, or more different HLA haplotypes.

In some embodiments, all the CD4⁺ T cells in the population have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other. In some embodiments, all the CD4⁺ T cells in the population have at least 1/8, 2/8, 3/8, 4/8, 5/8, 6/8, 7/8, or 8/8 match at the HLA-A, HLA-B, HLA-C, and HLA-DRB1 loci to each other. In some embodiments, all the CD4⁺ T cells in the population have 2/2 match at the HLA-A locus to each other. In some embodiments, all the CD4⁺ T cells in the population have 2/2 match at the HLA-B locus to each other. In some embodiments, all the CD4⁺ T cells in the population have 2/2 match at the HLA-C locus to each other. In some embodiments, all the CD4⁺ T cells in the population have at least 3/4 or 4/4 match at the HLA-DRB1 and HLA-DQB1 loci with each other. In some embodiments, all the CD4⁺ T cells in the population have an A*02 or A*24 allele.

In some embodiments, all the CD4⁺ T cells in the population have less than 5/10, 6/10, 7/10, 8/10, or 9/10 match at the HLA-A, HLA-B, HLA-C, HLA-DRB1, and HLA-DQB1 loci to each other. In some embodiments, all the CD4⁺ T cells in the population have less than 4/8, 5/8, 6/8, 7/8, or 8/8 match at the HLA-A, HLA-B, HLA-C, and HLA-DRB1 loci to each other. In some embodiments, all the CD4⁺ T cells in the population have less than 2/2 match at the HLA-A locus to each other. In some embodiments, all the CD4⁺ T cells in the population have less than 2/2 match at the HLA-B locus to each other. In some embodiments, all the CD4⁺ T cells in the population have less than 2/2 match at the HLA-C locus to each other. In some embodiments, all the CD4⁺ T cells in the population have less than 3/4 or 4/4 match at the HLA-DRB1 and HLA-DQB1 loci with each other.

In some embodiments, all the CD4⁺ T cells in the population have no match at the HLA-A, HLA-B, HLA-C, HLA-DRB1, and HLA-DQB1 loci to each other. In some embodiments, all the CD4⁺ T cells in the population have no match at the HLA-A, HLA-B, HLA-C, and HLA-DRB1 loci to each other. In some embodiments, all the CD4⁺ T cells in the population have no match at the HLA-A locus to each other. In some embodiments, all the CD4⁺ T cells in the population have no match at the HLA-B locus to each other. In some embodiments, all the CD4⁺ T cells in the population have no match at the HLA-C locus to each other. In some embodiments, all the CD4⁺ T cells in the population have no match at the HLA-DRB1 and HLA-DQB1 loci with each other.

In some embodiments, none of the CD4⁺ T cells is immortalized. In some embodiments, the exogenous polynucleotide comprises an IL-10-encoding polynucleotide segment operably linked to expression control elements. In some embodiments, the IL-10 is a human IL-10. In some embodiments, the IL-10 is a viral IL-10. In some embodiments, the IL-10 is a variant of human IL-10 having the sequence of human IL-10 with one, two, three, four, five, six, seven, eight, nine or ten amino acid modifications. In some embodiments, the one, two, three, four, five, six, seven, eight, nine or ten amino acid modifications are substitution with amino acid(s) of viral IL-10 at corresponding amino acid position(s). In some embodiment, the variant of human IL-10 has the sequence of SEQ ID NO: 8 or 9.

In some embodiments, the IL-10-encoding polynucleotide segment encodes a protein having the sequence of SEQ ID NO:1. In some embodiments, the IL-10-encoding polynucleotide segment has the sequence of SEQ ID NO:2. In some embodiments, the expression control elements drive constitutive expression of the encoded IL-10. In some embodiments, the expression control elements drive expression of IL-10 in activated CD4⁺ T cells. In some embodiments, the expression control elements drive tissue-specific or CD4⁺ T cell-specific expression.

In some embodiments, the exogenous polynucleotide further comprises a sequence encoding a selection marker. In some embodiments, the selection marker is ΔNGFR. In some embodiments, the ΔNGFR has the sequence of SEQ ID NO: 3. In some embodiments, the exogenous polynucleotide comprises a sequence of SEQ ID NO:4. In some embodiments, the exogenous polynucleotide having a sequence of SEQ ID NO: 5.

In some embodiments, the selection marker is a truncated EGFR polypeptide. In some embodiments, the selection marker is a truncated human EGFR polypeptide.

In some embodiments, the exogenous polynucleotide is integrated into the T cell nuclear genome. In some embodiments, the exogenous polynucleotide is not integrated into the T cell nuclear genome. In some embodiments, the exogenous polynucleotide further comprises lentiviral vector sequences. In some embodiments, the exogenous polynucleotide is not integrated into the T cell nuclear genome.

In some embodiments, at least 70% of the CD4⁺ T cells within the population express IL-10. In some embodiments, at least 90% of the CD4⁺ T cells within the population express IL-10. In some embodiments, at least 95%, 98% or 99% of the CD4⁺ T cells within the population express IL-10. In some embodiments the selection marker is ΔNGFR. In some embodiments, expression level of IL-10 linearly correlates with expression level of the selection marker. In the case, IL-10 expression level can be determined by the expression level of the selection marker.

In some embodiments, the genetically modified CD4⁺ T cells constitutively express at least 100 pg IL-10 per 10⁶ of the CD4⁺ T cells/mL of culture medium. In some embodiments, the genetically modified CD4⁺ T cells constitutively express at least 200 pg, 500 pg, 1 ng, 5 ng, 10 ng, or 50 ng IL-10 per 10⁶ of the CD4⁺ T cells/mL. In some embodiments, the genetically modified CD4⁺ T cells express at least 1 or 2 ng IL-10 per 10⁶ of the CD4⁺ T cells/mL after activation with anti-CD3 and anti-CD28 antibodies. In some embodiments, the genetically modified CD4⁺ T cells express at least 2 ng, 5 ng, 10 ng, 100 ng, 200 ng, or 500 ng IL-10 per 10⁶ of the CD4⁺ T cells/mL after activation with anti-CD3 and anti-CD28 antibodies. In some embodiments, the genetically modified CD4⁺ T cells express IL-10 at a level at least 5-fold higher than unmodified CD4⁺ T cells. In some embodiments, the genetically modified CD4⁺ T cells express IL-10 at a level at least 10-fold higher than unmodified CD4⁺ T cells.

In some embodiments, at least 70% of the CD4⁺ T cells within the population express the selection marker from the exogenous polynucleotide. In some embodiments, at least 90% of the CD4⁺ T cells within the population express the selection marker from the exogenous polynucleotide. In some embodiments, at least 95%, 98% or 99% of the CD4⁺ T cells within the population express the selection marker from the exogenous polynucleotide.

In some embodiments, the genetically modified CD4⁺ T cells express CD49b. In some embodiments, the genetically modified CD4⁺ T cells express LAG-3. In some embodiments, the genetically modified CD4⁺ T cells express TGF-β. In some embodiments, the genetically modified CD4⁺ T cells express IFN-γ. In some embodiments, the genetically modified CD4⁺ T cells express granzyme B (GzB). In some embodiments, the genetically modified CD4⁺ T cells express perforin. In some embodiments, the genetically modified CD4⁺ T cells express CD18. In some embodiments, the genetically modified CD4⁺ T cells express CD2. In some embodiments, the genetically modified CD4⁺ T cells express CD226. In some embodiments, the genetically modified CD4⁺ T cells express IL-22.

In some embodiments, the CD4⁺ T cells have not been anergized in the presence of peripheral blood mononuclear cells (PBMCs) from a host. In some embodiments, the CD4⁺ T cells have not been anergized in the presence of recombinant IL-10 protein, wherein the recombinant IL-10 protein is not expressed from the CD4⁺ T cells. In some embodiments, the CD4⁺ T cells have not been anergized in the presence of DC10 cells from a host.

In some embodiments, the CD4⁺ T cells are in a frozen suspension. In some embodiments, the CD4⁺ T cells are in a liquid suspension. In some embodiments, the liquid suspension has previously been frozen.

In another aspect of the present disclosure provides a pharmaceutical composition comprising:
(i) the population of CD4⁺ T cells described herein; suspended in
(ii) a pharmaceutically acceptable carrier.

In yet another aspect, the present disclosure provides a method of making polydonor CD4^{IL-10} cells, comprising the steps of:
(i) pooling primary CD4⁺ T cells obtained from at least two different T cell donors; and
(ii) modifying the pooled CD4⁺ T cells by introducing an exogenous polynucleotide encoding IL-10,
thereby obtaining the genetically-modified CD4⁺ T cells.

In one aspect, the present disclosure provides a method of making polydonor CD4^{IL-10} cells, comprising the steps of:
(i) obtaining primary CD4⁺ T cells from at least two different T cell donors; and
(ii) separately modifying each donor's CD4⁺ T cells by introducing an exogenous polynucleotide encoding IL-10, and then
(iii) pooling the genetically modified CD4⁺ T cells, thereby obtaining the genetically-modified CD4⁺ T cells.

In some embodiments, the method further comprises the step, after step (i) and before step (ii), after step (ii), after step (ii) and before step (iii), or after step (iii), of:
incubating the primary CD4⁺ T cells in the presence of an anti-CD3 antibody, and anti-CD28 antibody or anti-CD3 antibody and CD28 antibody coated beads. In some embodiments, polydonor CD4^{IL-10} T cells have been cultured in the presence of T Cell TransAct™ from Miltenyi Biotec. In some embodiments, polydonor CD4^{IL-10} T cells have been cultured in the presence of ImmunoCult Human T Cell Activator™ from STEMCELL Technologies.

In some embodiments, the method comprises incubating the primary CD4⁺ T cells further in the presence of IL-2. In some embodiments, the exogenous polynucleotide is introduced into the primary CD4⁺ T cells using a viral vector. In some embodiments, the viral vector is a lentiviral vector. In some embodiments, the viral vector is a chimeric viral vector. In some embodiments, the viral vector is an adeno-associated viral vector. In some embodiments, the exogenous polynucleotide comprises a segment encoding IL-10 having the sequence of SEQ ID NO:1. In some embodiments, the IL-10-encoding polynucleotide segment has the sequence of SEQ ID NO:2.

In some embodiments, the exogenous polynucleotide further comprises a segment encoding a selection marker. In some embodiments, the encoded selection marker is ΔNGFR. In some embodiments, the encoded selection marker has the sequence of SEQ ID NO:3. In some embodiments, the encoded selection marker is a truncated EGFR polypeptide. In some embodiments, the encoded selection marker is a truncated human EGFR polypeptide.

In some embodiments, the method further comprises the step, after step (ii), of:
isolating the genetically-modified CD4⁺ T cells expressing the selection marker, thereby generating an enriched population of genetically-modified CD4⁺ T cells.

In some embodiments, at least 70% of the genetically-modified CD4⁺ T cells in the enriched population express IL-10. In some embodiments, at least 90%, 95%, or 98% of the genetically-modified CD4⁺ T cells in the enriched population express IL-10. In some embodiments, at least 70% of the genetically-modified CD4⁺ T cells in the enriched population express the selection marker. In some embodiments, at least 90%, 95%, or 98% of the genetically-modified CD4⁺ T cells in the enriched population express the selection marker.

In some embodiments, the method further comprises the step of incubating the enriched population of genetically-modified CD4⁺ T cells. In some embodiments, the step of incubating the enriched population of genetically-modified CD4⁺ T cells is performed in the presence of anti-CD3 antibody and anti-CD28 antibody or CD3 antibody and CD28 antibody coated beads in the presence of IL-2.

In some embodiments, the method further comprises the later step of freezing the genetically-modified CD4⁺ T cells. In some embodiments, in step (i), the primary CD4⁺ T cells are obtained from two, three, four, five, six, seven, eight, nine, or ten different T cell donors. In some embodiments, the at least two T cell donors have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other. In some embodiments, the at least two T cell donors have at least 1/8, 2/8, 3/8, 4/8, 5/8, 6/8, 7/8, or 8/8 match at the HLA-A, HLA-B, HLA-C, and HLA-DRB1 loci to each other. In some embodiments, the at least two T cell donors have 2/2 match at the HLA-A locus to each other. In some embodiments, the at least two T cell donors have 2/2 match at the HLA-B locus to each other. In some embodiments, the at least two T cell donors have 2/2 match at the HLA-C locus to each other. In some embodiments, the at least two T cell donors have at least 3/4 or 4/4 match at the HLA-DRB1 and HLA-DQB1 loci to each other. In some embodiments, each of the at least two T cell donors has an A*02 or A*24 allele.

In some embodiments, the at least two T cell donors have less than 5/10, 6/10, 7/10, 8/10, or 9/10 match at the HLA-A, HLA-B, HLA-C, HLA-DRB1, and HLA-DQB1 loci to each other. In some embodiments, the at least two T cell donors have less than 4/8, 5/8, 6/8, 7/8, or 8/8 match at the HLA-A, HLA-B, HLA-C, and HLA-DRB1 loci to each other. In some embodiments, the at least two T cell donors have less than 2/2 match at the HLA-A locus to each other. In some embodiments, the at least two T cell donors have less than 2/2 match at the HLA-B locus to each other. In some embodiments, the at least two T cell donors have less than 2/2 match at the HLA-C locus to each other. In some embodiments, the at least two T cell donors have less than 3/4 or 4/4 match at the HLA-DRB1 and HLA-DQB1 loci to each other.

In some embodiments, in step (i), the primary CD4⁺ T cells are obtained from one or more frozen stocks. In some embodiments, in step (i), the primary CD4⁺ T cells are obtained from unfrozen peripheral blood mononuclear cells of the at least two different T cell donors.

In some embodiments, the method further comprises the step of isolating CD4⁺ T cells from the peripheral blood mononuclear cells. In some embodiments, the peripheral blood mononuclear cells are obtained from buffy coat or apheresis.

In another aspect, the present disclosure provides method of treating a patient, comprising the step of:
administering the polydonor CD4^{IL-10} cells or the pharmaceutical composition of the present disclosure to a patient in need of immune tolerization.

In some embodiments, the method further comprises the preceding step of thawing a frozen suspension of polydonor CD4^{IL-10} cells.

In some embodiments, the polydonor CD4^{IL-10} cells or the pharmaceutical composition prevents or reduces severity of pathogenic T cell response in the patient.

In some embodiments, the polydonor CD4^{IL-10} cells or the pharmaceutical composition prevents or reduces the severity of an inflammatory or autoimmune response.

In some embodiments, the method further comprises the step of administering mononuclear cells from a hematopoietic stem cells (HSC) donor to the patient. In some embodiments, the polydonor CD4^{IL-10} cells or the pharmaceutical composition and the mononuclear cells from a HSC donor are administered concurrently. In some embodiments, the mononuclear cells from a HSC donor are administered either prior to or subsequent to administration of the polydonor CD4^{IL-10} cells or the pharmaceutical composition. In some embodiments, the mononuclear cells are in the PBMC. In some embodiments, the mononuclear cells are in the bone marrow. In some embodiments, the mononuclear cells are in the cord blood. In some embodiments, the mononuclear cells have been isolated from the PBMC, bone marrow or cord blood.

In some embodiments, the method further comprises the step of:
administering hematopoietic stem cells (HSC) of an HSC donor to the patient either prior to or subsequent to administration of the polydonor CD4^{IL-10} cells or pharmaceutical composition.

In some embodiments, the HSC donor is partially HLA-mismatched to the patient. In some embodiments, the HSC donor has less than 5/10, 6/10, 7/10, 8/10, 9/10 or 10/10 match at the HLA-A, HLA-B, HLA-C, HLA-DRB1, and HLA-DQB1 loci to the patient. In some embodiments, the HSC donor has less than 4/8, 5/8, 6/8, 7/8, or 8/8 match at the HLA-A, HLA-B, HLA-C, and HLA-DRB1 loci to the patient. In some embodiments, the HSC donor has less than 2/2 match at the HLA-A, HLA-B, or HLA-C locus to the patient. In some embodiments, the HSC donor has less than 3/4 or 4/4 match at the HLA-DRB1 and HLA-DQB1 loci to the patient.

In some embodiments, one or more of the T cell donors are HLA-mismatched or partially HLA-mismatched to the patient. In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the patient. In some embodiments, one or more of the T cell donors have less than 4/8, 5/8, 6/8, 7/8, or 8/8 match at the HLA-A, HLA-B, HLA-C, and HLA-DRB1 loci to the patient. In some embodiments, one or more of the T cell donors have less than 2/2 match at the HLA-A, HLA-B, or HLA-C locus to the patient. In some embodiments, one or more of the T cell donors have less than 2/4, 3/4 or 4/4 match at the HLA-DRB1 and HLA-DQB1 loci to the patient. In some embodiments, one or more of the T cell donors are HLA-mismatched or partially HLA-mismatched with the HSC donor. In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the HSC donor. In some embodiments, one or more of the T cell donors have less than 4/8, 5/8, 6/8, 7/8, or 8/8 match at the HLA-A, HLA-B, HLA-C, and HLA-DRB1 loci to the HSC donor. In some embodiments, one or more of the T cell donors have less than 2/2 match at the HLA-A, HLA-B, or HLA-C locus to the HSC donor. In some embodiments, one or more of the T cell donors have less than 3/4 or 4/4 match at the HLA-DRB1 and HLA-DQB1 loci to the HSC donor.

In some embodiments, the polydonor CD4^{IL-10} cells or the pharmaceutical composition prevents or reduces severity of GvHD by the transplanted hematopoietic stem cells.

In some embodiments, the polydonor CD4^{IL-10} cells or the pharmaceutical composition prevents or reduces severity of pathogenic response of lymphoid cells present in the transplanted hematopoietic stem cells population.

In some embodiments, the patient has a cancer. In some embodiments, the patient has neoplastic cells. In some embodiments, the neoplastic cells express CD13, HLA-class I and CD54. In some embodiments, the neoplastic cells express CD112, CD58, or CD155.

In some embodiments, the patient has a cancer. In some embodiments, the cancer is a solid or hematological neoplasm. In some embodiments, the patient has a cancer selected from the group consisting of: Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Acute Lymphocytic (ALL), Acute Myeloid (AML, including myeloid sarcoma and leukemia cutis), Chronic Lymphocytic (CLL), Chronic Myeloid (CML) Leukemia, Chronic Myelomonocytic (CMML), Leukemia in Children, Liver Cancer, Lung Cancer, Lung Cancer with Non-Small Cell, Lung Cancer with Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

In some embodiments, the cancer is a myeloid cancer. In some embodiments, the cancer is AML or CML.

In some embodiments, the patient has an inflammatory or autoimmune disease. In some embodiments, the inflammatory or autoimmune disease is selected from the group consisting of: type-1 diabetes, autoimmune uveitis, autoimmune hepatitis, vitiligo, alopecia areata, rheumatoid arthritis, psoriasis, psoriatic arthritis, multiple sclerosis, systemic lupus, inflammatory bowel disease, Addison's disease, Graves' disease, Sjögren's syndrome, Hashimoto's thyroiditis, myasthenia gravis, autoimmune vasculitis, pernicious anemia, ulcerative colitis, bullous diseases, scleroderma, Crohn's disease, celiac disease and celiac disease.

In some embodiments, the inflammatory or autoimmune disease is Crohn's disease, ulcerative colitis, celiac disease, type-1 diabetes, lupus, psoriasis, psoriatic arthritis, or rheumatoid arthritis.

In some embodiments, the patient has a disease or disorder involving hyperactivity of NLPR3 inflammasome. In some embodiments, the patient has type 2 diabetes, neurodegenerative diseases, cardiovascular—or inflammatory bowel disease.

In some embodiments, the patient has a disease or disorder involving increased IL-1 production by activated monocytes, macrophages or dendritic cells.

In some embodiments, the patient has a disease or disorder involving increased IL-18 production by activated monocytes, macrophages or dendritic cells.

In some embodiments, the patient has a disease or disorder involving increased mature caspase 1 production by activated monocytes, macrophages or dendritic cells.

In some embodiments, the patient has an allergic or atopic disease. In some embodiments, the allergic or atopic disease is selected from the group consisting of: asthma, atopic dermatitis, and rhinitis. In some embodiments, the patient has a food allergy.

In some embodiments, the method further comprises the step of cell and organ transplantation to the patient, either prior to or subsequent to administration of the population of CD4⁺ T cells or the pharmaceutical composition. In some embodiments, the polydonor CD4^{IL-10} cells or the pharmaceutical composition prevents or reduces severity of host rejection of the cell and organ transplants.

In some embodiments, the method further comprises the step of transplanting iPS cell-derived cells or tissues to the patient, either prior to or subsequent to administration of the population of CD4⁺ T cells or the pharmaceutical composition.

In some embodiments, polydonor CD4^{IL-10} cells or the pharmaceutical composition prevents or reduces severity of host rejection of the transplantation.

In some embodiments, the method further comprises the step of administering a recombinant Adenovirus, Adeno-Associated Virus (AAV), Herpes simplex virus (HSV), Retrovirus, Lentivirus, Alphavirus, Flavivirus, Rhabdovirus, Measles virus, Newcastle disease Virus, Poxvirus, or Picornavirus to the patient, either prior to or subsequent to administration of the polydonor CD4^{IL-10} cells or the pharmaceutical composition. In some embodiments, the polydonor CD4^{IL-10} cells or the pharmaceutical composition reduces immune responses against the recombinant Adenovirus, Adeno-Associated Virus (AAV), Herpes simplex virus (HSV), Retrovirus, Lentivirus, Alphavirus, Flavivirus, Rhabdovirus, Measles virus, Newcastle disease Virus, Poxvirus, or Picornavirus.

In some embodiments, the patient has an excessive immune response against viral or bacterial infection. In some embodiments, the patient has a coronavirus infection. In some embodiments, the patient has organ and/or tissue damage.

In some embodiments, the method further comprises the step of administering an immunogenic therapeutic protein to the patient, either prior to or subsequent to administration of the population of polydonor CD4^{IL-10} cells or the pharmaceutical composition. In some embodiments, the population of polydonor CD4^{IL-10} cells, or the pharmaceutical composition reduces immune responses against the immunogenic therapeutic protein. In some embodiments, the immunogenic therapeutic protein is selected from a therapeutic antibody, a factor VIII replacement, a cytokine, and a cytokine mutein.

In some embodiments, the method further comprises the step of detecting the selection marker in a biological sample obtained from the patient, thereby detecting presence or absence of polydonor CD4^{IL-10} T cells. In some embodiments, the biological sample is a biopsy or blood from the patient.

In one aspect, the present disclosure provides a method of treating a patient with a malignancy, comprising: administering an allo-HSCT to the patient, and administering a therapeutically effective amount of polydonor CD4^{IL-10} cells. In some embodiments, the allo-HSCT is administered prior to administration of the polydonor CD4^{IL-10} cells. In some embodiments, the allo-HSCT is administered after administration of the polydonor CD4^{IL-10} cells.

In some embodiments, none of the donors of the CD4^{IL-10} cells in the polydonor CD4^{IL-10} cells is the donor of the HSCT.

In another aspect, the present disclosure provides a method of treating a hematological cancer, comprising: administering to a hematological cancer patient an amount of polydonor CD4^{IL-10} cells sufficient to induce anti-cancer effects, wherein the polydonor CD4^{IL-10} cells comprise CD4⁺ T cells that have been obtained from at least two different T cell donors and then genetically modified by vector-mediated gene transfer of the coding sequence of human IL-10 under control of a constitutive or inducible promoter.

In some embodiments, the method of treating a hematological cancer comprises the step wherein the administered polydonor CD4⁺ T cells that are sufficient to induce anti-cancer effects have been obtained from the individual donors and are first separately genetically modified by vector-mediated gene transfer of the coding sequence of human IL-10 under the control of a constitutive or inducible promoter and then pooled.

In some embodiments, the method of treating a hematological cancer comprises the step wherein the administered polydonor CD4⁺ T cells that are sufficient to induce anti-cancer effects have been obtained from the individual donors are first pooled and then the pool is genetically modified by vector-mediated gene transfer of the coding sequence of human IL-10 under the control of a constitutive or inducible promoter.

In some embodiments, the method further comprises the step of administering allo HSCT to the patient prior to or subsequence to administration of the polydonor CD4^{IL-10} cells.

In some embodiments, the amount of polydonor CD4^{IL-10} cells is further sufficient to suppress or prevent graft-versus-host disease (GvHD) without suppressing graft-versus-leukemia (GvL) or graft-versus-tumor (GvT) efficacy of the allo HSCT.

In some embodiments, the hematological cancer is a myeloid leukemia.

In some embodiments, the polydonor CD4^{IL-10} cells target and kill cancer cells that express CD13. In some embodiments, the polydonor CD4^{IL-10} cells target and kill cancer cells that express HLA-class I. In some embodiments, the myeloid leukemia is acute myeloid leukemia (AML).

In some embodiments, the allo-HSCT is obtained from a related or unrelated donor with respect to the recipient. In some embodiments, the polydonor CD4^{IL-10} cells are non-autologous to the recipient. In some embodiments, the polydonor CD4^{IL-10} cells are allogeneic to the recipient. In some embodiments, the polydonor CD4^{IL-10} cells are not anergized to host allo-antigens prior to administration to the host.

In some embodiments, the polydonor CD4^{IL-10} cells are Tr1-like cells.

In some embodiments, the polydonor CD4^{IL-10} cells are polyclonal. In some embodiments, the polydonor CD4^{IL-10} cells are polyclonal and non-autologous to the recipient.

In some embodiments, the polydonor CD4^{IL-10} cells are isolated from at least two donors prior to being genetically modified. In some embodiments, none of the at least two donors is the same donor as the allo-HSCT donor. In some embodiments, the allo-HSCT is obtained from a matched or mismatched donor with respect to the recipient.

In some embodiments, the polydonor CD4^{IL-10} cells target and kill cells that express CD54. In some embodiments, the polydonor CD4^{IL-10} cells target and kill cancer cells that express HLA-class I and CD54. In some embodiments, the polydonor CD4^{IL-10} cells target and kill cancer cells that express CD112. In some embodiments, the polydonor CD4^{IL-10} cells target and kill cancer cells that express CD58. In some embodiments, the polydonor CD4^{IL-10} cells target and kill cancer cells in the host.

One aspect of the present disclosure provides a method of treating a hematological cancer by allogeneic hematopoietic stem cell transplant (allo-HSCT), comprising:
administering allo-HSCT to a subject (host);
administering to the host an amount of polydonor CD4^{IL-10} cells sufficient to suppress or prevent graft-versus-host disease (GvHD) without suppressing graft-versus-leukemia (GvL) or graft-versus-tumor (GvT) efficacy of the allo-HSCT graft;
wherein the polydonor CD4^{IL-10} cells comprise CD4⁺ T cells obtained from at least two different T cell donors and that are genetically modified by vector-mediated gene transfer of the coding sequence of human IL-10 under control of a constitutive or inducible promoter;
wherein the polydonor CD4^{IL-10} cells are non-autologous to the host and non-autologous to the allo-HSCT donor;
wherein the polydonor CD4^{IL-10} cells are not anergized to host allo-antigens prior to administration to the host; and
wherein the polydonor CD4^{IL-10} cells are polyclonal and Tr1-like.

In some embodiments, the allo-HSCT is administered prior to administration of the polydonor CD4^{IL-10} cells. In some embodiments, the allo-HSCT is administered after administration of the polydonor CD4^{IL-10} cells.

Another aspect of the present disclosure provides a method of treating a hematological cancer by allogeneic hematopoietic stem cell transplant (allo-HSCT), comprising:
administering allo-HSCT to a subject (host);
administering to the host an amount of polydonor CD4^{IL-10} cells sufficient to suppress or prevent graft-versus-host disease (GvHD) without suppressing graft-versus-leukemia (GvL) or graft-versus-tumor (GvT) efficacy of the allo-HSCT;
wherein the polydonor CD4^{IL-10} cells comprise CD4⁺ T cells obtained from at least two different T cell donors and genetically modified by vector-mediated gene transfer of the coding sequence of human IL-10 under control of a constitutive promoter;
wherein the polydonor CD4^{IL-10} cells target and kill cancer cells in the host;
wherein the polydonor CD4^{IL-10} cells are not anergized to host allo-antigens prior to administration to the host; and
wherein all of the polydonor CD4^{IL-10} cells are non-autologous to the host, and polyclonal, and are Tr1-like.

Another aspect of the present disclosure provides CD4^{IL-10} cells from a single donor or multiple donors, where the IL-10 is viral IL-10. The viral IL-10 having the sequence of SEQ ID NO: 6, 19, 20, or 21. In some embodiments, the viral IL-10 is encoded by a polynucleotide having the sequence of SEQ ID NO: 7. In some embodiments, the IL-10 is human IL-10 where one, two, three, four, five, six, seven, eight, nine or ten amino-acid from human IL-10 are replaced by the corresponding amino-acid sequence from viral IL-10. In some embodiments, the CD4⁺ T cells are transduced with exogenous viral IL-10 under the control of constitutive promoter. In some embodiments, the expression control elements drive expression of viral IL-10 in activated CD4⁺ T cells. In some embodiments, the exogenous polynucleotide encoding viral IL-10 is integrated into the T cell nuclear genome. In some embodiments, the exogenous polynucleotide encoding viral IL-10 is not integrated into the T cell nuclear genome. In some embodiments, the exogenous polynucleotide encoding viral IL-10 has the sequence of SEQ ID NO: 7.

Another aspect of the present disclosure provides CD4^{IL-10} cells from a single donor or multiple donors, where the IL-10 is IL-10 of a mouse (SEQ ID NO: 10), rat (SEQ ID NO: 11), Macaca mulatta (MACMU) (SEQ ID NO: 12), gorilla (SEQ ID NO: 13), cynomolgus monkey (CYNO) (SEQ ID NO: 14), olive baboon (SEQ ID NO: 15), bonobo (SEQ ID NO: 16), chimpanzee (SEQ ID NO: 17), or EBVB9 (SEQ ID NO: 18). In some embodiments, the IL-10 is a protein having at least 90%, 95%, 98%, or 99% sequence identity to IL-10 of a mouse (SEQ ID NO: 10), rat (SEQ ID NO: 11), macaca mulatta (MACMU) (SEQ ID NO: 12), gorilla (SEQ ID NO: 13), cynomolgus monkey (CYNO) (SEQ ID NO: 14), olive baboon (SEQ ID NO: 15), bonobo (SEQ ID NO: 16), chimpanzee (SEQ ID NO: 17), or EBVB9 (SEQ ID NO: 18).

Another aspect of the present disclosure provides CD4^{IL-10} cells from a single donor or multiple donors, where the IL-10 is a variant of human IL-10. The variant of human IL-10 having the sequence of SEQ ID NO: 19 or SEQ ID NO: 20. In some embodiments, the IL-10 is human IL-10 where one, two, three, four, five, six, seven, eight, nine or ten amino-acid from human IL-10 are replaced by the corresponding amino-acid sequence from IL-10 of another species (e.g., IL-10 of a mouse (SEQ ID NO: 10), rat (SEQ ID NO: 11), macaca mulatta (MACMU) (SEQ ID NO: 12), gorilla (SEQ ID NO: 13), cynomolgus monkey (CYNO) (SEQ ID NO: 14), olive baboon (SEQ ID NO: 15), bonobo (SEQ ID NO: 16), chimpanzee (SEQ ID NO: 17), or EBVB9 (SEQ ID NO: 18). In some embodiments, the CD4⁺ T cells are transduced with exogenous the IL-10 variant under the control of constitutive promoter. In some embodiments, the expression control elements drive expression of the IL-10 variant in activated CD4⁺ T cells. In some embodiments, the exogenous polynucleotide encoding the IL-10 variant is integrated into the T cell nuclear genome. In some embodiments, the exogenous polynucleotide encoding the IL-10 variant is not integrated into the T cell nuclear genome.

In yet another aspect, the present disclosure provides a method of making viral IL-10 CD4^{IL-10}, comprising the steps of:
(i) obtaining primary CD4⁺ T cells from a single T cell donor; and
(ii) modifying the donor CD4⁺ T cells by introducing an exogenous polynucleotide encoding viral IL-10,
thereby obtaining the genetically-modified CD4⁺ T cells.

In some embodiments, the method further comprises the step, after step (i), or after step (ii), of: incubating the primary CD4⁺ T cells in the presence of an anti-CD3 antibody, and anti-CD28 antibody or anti-CD3 antibody and CD28 antibody coated beads.

In some embodiments, the method comprises incubating the primary CD4⁺ T cells further in the presence of IL-2. In some embodiments, the exogenous polynucleotide encoding viral IL-10 using a vector.

In some embodiments, the exogenous polynucleotide encoding viral IL-10 comprises a segment encoding a selection marker. In some embodiments, the encoded selection marker is ΔNGFR. In some embodiments, the encoded selection marker has the sequence of SEQ ID NO:3. In some embodiments, the encoded selection marker is a truncated EGFR polypeptide. In some embodiments, the encoded selection marker is a truncated human EGFR polypeptide.

In some embodiments, the method further comprises the step, after step (ii), of: isolating the genetically-modified CD4⁺ T cells expressing the selection marker, thereby generating an enriched population of genetically-modified CD4⁺ T cells.

In some embodiments, the method further comprises the step of incubating the enriched population of genetically-modified CD4⁺ T cells. In some embodiments, the step of incubating the enriched population of genetically-modified CD4⁺ T cells is performed in the presence of anti-CD3 antibody and anti-CD28 antibody or CD3 antibody and CD28 antibody coated beads in the presence of IL-2.

In some embodiments, in step (i), the primary CD4⁺ T cells are obtained from frozen stock. In some embodiments, in step (i), the primary CD4⁺ T cells are obtained from unfrozen peripheral blood mononuclear cells of the single T cell donor.

In some embodiments, the method further comprises the step of isolating CD4⁺ T cells from the peripheral blood mononuclear cells. In some embodiments, the peripheral blood mononuclear cells are obtained from buffy coat or apheresis.

### 5. BRIEF DESCRIPTION OF THE DRAWINGS

FIG. 1 is a non-limiting illustration of the structure of a bidirectional lentiviral vector for delivering human IL-10 and ΔNGFR coding sequences into CD4⁺ T cells from multiple donors to produce polydonor CD4^{IL-10} cells.

FIG. 2 illustrates the complete and circular structure of a bidirectional lentiviral vector for generating the lentiviral vector to deliver human IL-10 and ΔNGFR coding sequences into CD4⁺ T cells from multiple donors to produce polydonor CD4^{IL-10} cells.

FIG. 3 illustrates an exemplary protocol for generating CD4^{IL-10} cells.

FIG. 4A shows percentages of CD4⁺ΔNGFR*cells (mean±SD, n=10 left grey bar) and vector copy numbers (VCN, mean±SD, n=10 right grey bar) in human CD4⁺ T cells transduced with LV-IL-10/ΔNGFR (a bidirectional lentiviral vector encoding for human IL-10 and a truncated form the human NGF receptor). FIG. 4B shows FACS analysis of expression of CD4 and ΔNGFR in human CD4⁺ T cells from two representative donors (Donor B and Donor C) transduced with LV-IL-10/ΔNGFR and purified using anti-CD271 Microbeads.

FIG. 5 shows cytokine production profile of single donor CD4^{IL-10} cells after the second (TF2) and third (TF3) restimulation. The TF2 (left panel) and TF3 (right panel) CD4^{IL-10} cells were left unstimulated (as indicated by arrow) or stimulated with immobilized CD3 (10 μg/mL) and soluble CD28 mAb (1 μg/mL) for 48 hours. Culture supernatants were collected and levels of IL-10, IL-4, IL-5, IFN-γ and IL-22 were determined by ELISA. All samples were tested in triplicate. Mean±SD, n=8 donors tested are presented.

FIG. 6A shows the percentage of CD4^{IL-10} cells expressing granzyme B (GzB) after 2^{nd} round of stimulation (TF2) analyzed by FACS. Box and whiskers of n=7 different single donors are presented. FIG. 6B shows % dead cells when CD4^{IL-10} cells (10⁵/well) were co-cultured with K562 and ALL-CM cells (10⁵/well) at 1:1 ratio for 3 days. Box and whiskers represent data from n=4 donors and dots represent data from single donors.

FIGS. 7A and 7B show that single donor CD4^{IL-10} cells can suppress the proliferation of allogeneic CD4⁺ T cells. Allogeneic PBMC cells were labeled with eFluor® 670 (5×10⁴ cells/well) and stimulated with allogeneic mature dendritic (DC) cells (5×10³ cells/well) and soluble anti-CD3 mAbs in the absence or presence of CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD4⁺ΔNGFR⁻ T cells. FIG. 7A show results from Donor-C, Donor-E, and Donor-F and FIG. 7B show results from Donor-H, Donor-I and Donor-L. Percentages of proliferation and suppression are indicated. The suppression mediated by CD4^{IL-10} cells was calculated as follows: 100−([proliferation of responders in the presence of CD4^{IL-10} cells/proliferation of responders alone]×100).

FIGS. 8A and 8B show that single donor CD4^{IL-10} cells can suppress the proliferation of allogeneic CD8⁺ T cells. Allogeneic PBMC cells were labeled with eFluor® 670 (5×10⁴ cells/well) and stimulated with allogeneic mature dendritic (DC) cells (5×10³ cells/well) and soluble anti-CD3 mAbs in the absence or presence of CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD8⁺ΔNGFR⁻ T cells. FIG. 8A show results from Donor-C, Donor-E, and Donor-F and FIG. 8B show results from Donor-H, Donor-I and Donor-L. Percentages of proliferation and suppression are indicated. The suppression mediated by CD4^{IL-10} cells was calculated as follows: 100−([proliferation of responders in the presence of CD4^{IL-10} cells/proliferation of responders alone]×100).

FIG. 9 shows cytokine production profile of polydonor CD4^{IL-10} cells after third (TF3) restimulation, compared to mean levels (+/−SD) produced by CD4^{IL-10} cells from 8 individual donors. The TF3 CD4^{IL-10} cells from three donors were pooled at a 1:1:1 ratio and stimulated with immobilized CD3 (10 μg/mL) and soluble CD28 mAb (1 μg/mL) for 48 hours. Culture supernatants were collected and levels of IL-10, IL-4, IL-5, IFN-γ and IL-22 were determined by ELISA. Dots are results of polydonor CD4^{IL-10} cells; gray bars represent mean±SD, n=8 single donors.

FIG. 10A shows the percentage of polydonor CD4^{IL-10} cells expressing granzyme B (GzB) compared to mean % levels (+/−SD) of granzyme B expression by CD4^{IL-10} cells of n=3 single donors used to generate the pool. Cells were analyzed by FACS after the 3^{rd} round of stimulation (TF3). FIG. 10B shows % dead cells when polydonor CD4^{IL-10} cells (10⁵/well) were co-cultured with K562 and ALL-CM cells (10⁵/well) at 1:1 ratio for 3 days. Residual leukemic cells (CD45+, CD3−) were counted by FACS for each target cell. Dots are results of polydonor CD4^{IL-10} and gray bars represent mean±SD of n=3 single donors used to generate the pool.

FIGS. 11A and 11B show that polydonor CD4^{IL-10} cells can suppress the proliferation of allogeneic CD4⁺ T cells and CD8⁺ T cells. Allogeneic PBMC cells were labeled with eFluor® 670 (5×10⁴ cells/well) and stimulated with allogeneic mature dendritic (DC) cells (1×10⁴ cells/well) and soluble anti-CD3 mAbs in the absence or presence of polydonor CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD4⁺ΔNGFR⁻ T cells and CD8⁺ΔNGFR⁻ T cells. FIG. 11A shows results from polydonor CD4^{IL-10} cells containing CD4⁺ cells pooled from Donor-C, Donor-E, and Donor-F. FIG. 11B shows results from polydonor CD4^{IL-10} cells containing CD4⁺ cells pooled from Donor-H, Donor-I, and Donor-L. The suppression mediated by CD4^{IL-10} cells was calculated as follows: 100−([proliferation of responders in the presence of CD4^{IL-10} cells/proliferation of responders alone]×100).

FIG. 12 illustrates a protocol for testing induction of GvHD by human PBMC and/or polydonor CD4^{IL-10} (BC-C/E/F) cells injected on day 0 post-irradiation.

FIG. 13 shows % of NSG mice free of GvHD on each day after injection of PBMC (5×10⁶ cells/mouse), polydonor (three donors; BC-C/E/F) CD4^{IL-10} cells (5×10⁶ cells/mouse), or PBMC (5×10⁶ cells/mouse) in combination with polydonor CD4^{IL-10} cells (three donors; BC-C/E/F) (5×10⁶ cells/mouse).

FIG. 14 shows migration of CD4^{IL-10} cells to spleen (left panel) and bone marrow (right panel) in NSG mice injected with PBMC (5×10⁶ cells/mouse), polydonor (three donors; (BC-C/E/F)) CD4^{IL-10} cells (5×10⁶ cells/mouse), or PBMC (5×10⁶ cells/mouse) in combination with polydonor CD4^{IL-10} cells (three donors; (BC-C/E/F)) (5×10⁶ cells/mouse). Box and whiskers on n=8 tested animals are presented.

FIG. 15 illustrates a protocol for testing induction of GvHD by CD4⁺ T cells and polydonor (BC-H/I/L) or single-donor (BC-H) CD4^{IL-10} cells injected on day 3 post-irradiation.

FIG. 16 shows % of NSG mice free of GvHD on each day after injection.

FIGS. 17A-17C shows graft-versus-leukemia (GvL) effect tested based on reduction of circulating leukemia cells and long-term leukemia free survival. Leukemia was measured as previously described (Locafaro G. et al Molecular Therapy 2017). NSG mice were sub-lethally irradiated and intravenously injected with myeloid leukemia cells (ALL-CM) (2.5×10⁶) on day 0. FIG. 17A is an illustration of the experiment. FIG. 17B shows leukemia free survival rate in the animals injected with PBMC (2.5×10⁶) or single donor (from donor BC-I and donor BC-H) CD4^{IL-10} cells (2.5×10⁶) on day 3. FIG. 17C shows leukemia free survival rate in the animals injected with PBMC (2.5×10⁶) or polydonor CD4^{IL-10} cells (from donor BC-I and donor BC-H) (2.5×10⁶) on day 3.

FIG. 18A-18C show long-term leukemia free survival rate measured in NSG mice sub-lethally irradiated and intravenously injected with ALL-CM cells (2.5×10⁶) at day 0. FIG. 18A is an illustration of the experiment. FIG. 18B shows data from animals injected with mononuclear cells (PBMC) (2.5×10⁶) alone or mononuclear cells (PBMC) (2.5×10⁶)+single donor (from donor BC-H and donor BC-I) CD4^{IL-10} cells (2.5×10⁶) at day 3. FIG. 18C shows data from animals injected with mononuclear cells (PBMC) (2.5×10⁶) alone or mononuclear cells (PBMC) (2.5×10⁶)+polydonor CD4^{IL-10} cells (BC-I/H) (2.5×10⁶) at day 3.

FIG. 19A-19G show inhibition of NLPR3 inflammasome activation by CD4^{IL-10} cells. FIG. 19A shows the effect of CD4^{IL-10} cell supernatant from a single donor (#1) on the production of IL-1β by LPS activated monocytes. FIG. 19B shows the effect of CD4^{IL-10} cell supernatant from another single donor (#2) on the production of IL-1β by LPS activated monocytes. FIG. 19C shows the effect of CD4^{IL-10} cell supernatant from a single donor (#1) on the inhibition of LPS induced IL-1β production enhanced by NLPR3 inflammasome activator nigericine (NIG). FIG. 19D shows the effect of CD4^{IL-10} cell supernatant from a single donor (#2) on the inhibition of LPS induced IL-1β production enhanced by NLPR3 inflammasome activator nigericine (NIG). FIG. 19E is a bar graph that shows the effect of CD4^{IL-10} cell supernatant from a single donor (BC-E) and pooled cells from 2 different donors (BC-C/E) on LPS induced IL-1β production by monocytes in the presence or absence of anti-IL-10 receptor (anti-IL-10R) mAb. FIG. 19F shows the effect of polydonor CD4^{IL-10} cell (BC-T/U/V) supernatants on IL-1β production by monocytes in the presence or absence of anti-IL-10R mAb. FIG. 19G shows the effects of polydonor CD4^{IL-10} cell (BC-T/U/V) supernatants on IL-18 production induced by LPS in combination with nigericin in the presence or absence of anti-IL-10R mAb.

FIG. 20 illustrates an experimental protocol for testing graft versus myeloid leukemia and xeno-GvHD effects. NSG-mice were intravenously injected with ALL-CM cells (2.5×10⁶) on day 0. At day 3 the mice were divided into five groups and each group was treated with (i) none as a control, (ii) allogeneic mononuclear cells (PBMC); (iii) allogeneic PBMC and polydonor CD4^{IL-10} cells (BC-V/T/E, pooled 1:1:1); (iv) allogeneic PBMC and single-donor CD4^{IL-10} cells (BC-E); or (v) polydonor CD4^{IL-10} cells (BC-V/T/E) at concentrations as indicated in FIG. 20.

FIG. 21 is a bar graph depicting the cytokine secretion profiles of single-donor (BC-V, BC-T, BC-V, and BC-E) and polydonor CD4^{IL-10} cells (POOL: BC-E, BC-V and BC-T pooled 1:1:1).

FIG. 22 show suppressive effects of single-donor (BC-V and BC-E) and polydonor CD4^{IL-10} cells (pool of BC-V/T/E) on in vitro proliferation of allogeneic CD4+ and CD8+ T cells. Allogeneic PBMC cells were labeled with eFluor® 670 (5×10⁴ cells/well) and stimulated with allogenic mature dendritic (DC) cells (1×10⁴ cells/well) and soluble anti-CD3 mAbs in the absence or presence of CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD4⁺ΔNGFR⁻ (top) or CD8⁺ΔNGFR⁻ (bottom) T cells. FIG. 22 shows results from single donors BC-V and BC-E and pooled cells from donors BC-V/T/E. Percentages of proliferation and suppression are indicated. The suppression mediated by CD4^{IL-10} cells was calculated as follows: 100−([proliferation of responders in the presence of CD4^{IL-10} cells/proliferation of responders alone]×100).

FIG. 23 shows % of alive cells in a co-culture of single (BC-E and BC-V) or polydonor CD4^{IL-10} cells (BC-V/T/E) with ALL-CM myeloid tumor cells or K562 cells. The results show selective cytotoxic effect of single-donor and polydonor CD4^{IL-10} cells on ALL-CM myeloid tumor cells, but not on K562 cells which lack Class I MHC expression.

FIG. 24 shows leukemia-free survival rate measured in NSG-mice intravenously injected with ALL-CM cells (2.5×10⁶) on day 0. At day 3 the mice were divided into five groups and each group was treated with (i) none as a control; (ii) allogeneic mononuclear cells (PBMC); (iii) allogeneic PBMC and polydonor CD4^{IL-10} cells (BC-V/T/ET); (iv) allogeneic PBMC and single-donor CD4^{IL-10} cells (BC-E) or (v) polydonor CD4^{IL-10} cells (BC-V/T/E). The graph shows leukemia-free survival rate of animals in each group.

FIG. 25 shows % of NSG mice free of GvHD on each day following injection with ALL-CM cells (2.5×10⁶) and subsequent administration of (i) none as a control; (ii) allogeneic mononuclear cells (PBMC); (iii) allogeneic PBMC and polydonor CD4^{IL-10} cells (BC-V/T/E); (iv) allogeneic PBMC and single-donor CD4^{IL-10} cells (BC-E) or (v) polydonor CD4^{IL-10} cells were administered at day 3.

FIG. 26 shows that NSG mice dosed with 2.5E+06 of PBMC (allogeneic to the donors C, E, F and H) all succumbed to acute, and lethal xeno-GvHD at day 22. Administration of single-donor CD4^{IL-10} cells (lot C) or polydonor CD4^{IL-10} cells (lot CEFH) in combination with the PBMCs prevented the development of lethal xeno-GvHD in 75% (3/4 mice) and 80% (4/5 mice) of the mice, respectively. In contrast, transfer of 2.5E+06 polydonor CD4^{IL-10} cells did not induce any sign of GvHD. Taken together these results indicate that polydonor CD4^{IL-10} cells from 4 different donors suppress pathogenic human T cell responses as potently, or slightly more potently than single-donor CD4^{IL-10} cells. PBMC: peripheral mononuclear cells; GvHD: graft vs. host disease.

FIG. 27A shows alignment of IL-10 protein sequences of various species, including human (SEQ ID NO: 1), Mus musculus, “MOUSE” (SEQ ID NO: 10); Rattus norvegicus, “RAT” (SEQ ID NO: 11); Macaca mulatta, “MACMU” (SEQ ID NO: 12); Gorilla gorilla, “GORILLA” (SEQ ID NO: 13); Macaca fascicularis, “CYNO” (SEQ ID NO: 14); Papio anubis, “OLIVE BABOON” (SEQ ID NO: 15); Pan paniscus, “BONOBO” (SEQ ID NO: 16); Pan troglodytes, “CHIMP” (SEQ ID NO: 17); or EBVB9 (SEQ ID NO: 18).

FIG. 27B provides sequences of IL-10 variants generated by substituting one or more amino acids of human IL-10 by amino acids of viral IL-10 (EBVB9) at the corresponding positions. Also provided are sequences of the exemplary variants, possible huIL-10 hybrid #1 (SEQ ID NO: 19) and possible huIL-10 hybrid #2 (SEQ ID NO: 20). “*” indicates the one or more amino acid positions that are substituted. “#” indicates the preferred I105 to A105 amino acid substitution for IL-10 hybrid #2 (SEQ ID NO: 20).

FIG. 27C shows alignment of human IL-10 (SEQ ID NO: 1) with IL10 EBVB9 (SEQ ID NO: 18). “*” indicates the one or more amino acid positions that are substituted in IL-10 hybrid #1. “#” indicates the preferred I105 to A105 amino acid substitution for IL-10 hybrid #2.

The figures depict various embodiments of the present invention for purposes of illustration only. One skilled in the art will readily recognize from the following discussion that alternative embodiments of the structures and methods illustrated herein may be employed without departing from the principles of the invention described herein.

### 6. DETAILED DESCRIPTION

### 6.1. Definitions

Unless defined otherwise, all technical and scientific terms used herein have the meaning commonly understood by a person skilled in the art to which this invention belongs. As used herein, the following terms have the meanings ascribed to them below.

“Graft-versus-leukemia effect” or “GvL” refers to an effect that appears after allogeneic hematopoietic stem cell transplantation (HSCT) or bone marrow transplantation (BMT). T lymphocytes in the allogeneic graft eliminate malignant residual host leukemia cells.

“Graft versus tumor effect” or “GvT refers to an effect that appears after allogeneic hematopoietic stem cell transplantation (HSCT) or bone marrow transplantation (BMT). T lymphocytes in the allogeneic graft eliminate malignant residual host cancer cells, e.g., cells of myeloma and lymphoid and myeloid leukemias, lymphoma, multiple myeloma and possibly breast cancer. The term GvT is generic to GvL.

The terms “treatment”, “treating”, and the like are used herein in the broadest sense understood in the medical arts. In particular, the terms generally mean obtaining a desired pharmacologic and/or physiologic effect. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). Improvements in any conditions can be readily assessed according to standard methods and techniques known in the art. The population of subjects treated by the method of the disease includes subjects suffering from the undesirable condition or disease, as well as subjects at risk for development of the condition or disease.

“HLA-matched” as used herein refers to a pair of individuals having a matching HLA allele in the HLA class I (HLA-A, HLA-B, and HLA-C) and class II (HLA-DRB1 and HLA-DQB1) loci that allow the individuals to be immunologically compatible with each other. HLA compatibility can be determined using any of the methods available in the art, for example, as described in Tiervy, Haematologica 2016 Volume 101(6):680-687, which is incorporated by reference herein.

For a given locus, a pair of individuals have 2/2 match when each of two alleles of one individual match with the two alleles of the other individual. A pair of individuals have ½ match when only one of two alleles of one individual match with one of two alleles of the other individual. A pair of individuals have 10/10 match at the HLA-A, HLA-B, HLA-C, HLA-DRB1, and HLA-DQB1 loci when all of the ten alleles (two for each of the HLA-A, HLA-B, HLA-C, HLA-DRB1, and HLA-DQB1 loci) of one individual match with all ten alleles of the other individual.

In preferred embodiments, allele level typing is used for determination of HLA compatibility. Allele level typing corresponds to a unique nucleotide sequence for an HLA gene, as defined by using all digits in the first, second, third and fourth fields, e.g. A*02:01:01:01. Functionally, the third and fourth fields which characterize alleles that differ, respectively, by silent substitutions in the coding sequence and by substitutions in the non-coding sequence, are irrelevant, except when substitutions prevent the expression of HLA alleles (e.g. the null allele B*15:01:01:02N). Missing a null allele will lead to a mismatch that is very likely to be recognized by alloreactive T cells and have a deleterious clinical impact. Substitutions in non-coding sequences may influence the level of expression (e.g. the A24low allele A*24:02:01:02L). Such variability may also have an impact on anti-HLA allorecognition.

The term “HLA-mismatched” as used herein refers to a pair of individuals having a mis-matching HLA allele in the HLA class I (HLA-A, HLA-B, and HLA-C) and class II (HLA-DRB1 and HLA-DQB1) loci that make the individuals to be immunologically incompatible with each other.

The term “partially HLA-mismatched” as used herein refers to a pair of individuals having a mis-matching HLA allele in the HLA class I (HLA-A, HLA-B, and HLA-C) and class II (HLA-DRB1 and HLA-DQB1) loci that make the individuals to be immunologically incompatible with each other in a permissible degree. Some studies have identified permissive mismatches. Some HLA class I incompatibilities are considered to be more permissive.

“HLA haplotype” refers to a series of HLA loci-alleles by chromosome, one passed from the mother and one from the father. Genotypes for HLA class I (HLA-A, HLA-B, and HLA-C) and class II (HLA-DRB1 and HLA-DQB1) loci can be used to determine the HLA haplotype.

The term “therapeutically effective amount” is an amount that is effective to treat, and thus ameliorate a symptom of a disease.

The term “prophylactically effective amount” is an amount that is effect in terms of completely or partially preventing a disease, condition, or symptoms thereof The term “ameliorating” refers to any therapeutically beneficial result in the treatment of a disease state, e.g., a neurodegenerative disease state, including prophylaxis, lessening in the severity or progression, remission, or cure thereof.

### 6.2. Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### 6.3. Polydonor CD4^{IL-10} Cells

In a first aspect, a population of CD4⁺ T cells that have been genetically modified to comprise an exogenous polynucleotide encoding IL-10 is provided (CD4^{IL-10} cells). The population comprises CD4⁺ T cells obtained from at least two different T cell donors (polydonor CD4^{IL-10} cells).

### 6.3.1. CD4⁺ T Cells and T Cell Donors

CD4⁺ T cells used in polydonor CD4^{IL-10} populations can be isolated from peripheral blood, cord blood, or other blood samples from a donor, using methods available in the art. In typical embodiments, CD4⁺ T cells are isolated from peripheral blood, preferably a human donor. In certain embodiments, CD4⁺ T cells are isolated from peripheral blood by leukapheresis. In certain embodiments the CD4⁺ T cells are obtained from third party-blood banks. In certain embodiments the CD4+ T cells are obtained from buffy coats from centrifugation of whole blood.

In some embodiments, CD4⁺ T cells are isolated from a prior-frozen stock of blood or a prior-frozen stock of peripheral blood mononuclear cells (PBMCs). In some embodiments, CD4⁺ T cells are isolated from peripheral blood or from PBMCs that have not previously been frozen. In some embodiments, the CD4+ T cells are separately isolated from blood or PBMCs obtained from a plurality of donors, and then pooled. In some embodiments, the CD4+ T cells are isolated from blood or PBMCs that have first been pooled from a plurality of donors.

In some embodiments, the CD4⁺ T cells are obtained from three, four, five, six, seven, eight, nine, or ten different T cell donors.

In some embodiments, the at least two different T cell donors are selected without regard to genotype. In some embodiments, the at least two different T cell donors are selected based on genotype.

In certain embodiments, the at least two different T cell donors are selected based on their HLA haplotypes.

In some embodiments, some or all of the at least two different T cell donors have matching HLA haplotypes. In some embodiments, some or all of the at least two different T cell donors have a mis-matched HLA haplotype.

In some embodiments, all of the CD4⁺ T cells in the population have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other. In some embodiments, all of the CD4⁺ T cells in the population have at least 1/8, 2/8, 3/8, 4/8, 5/8, 6/8, 7/8, or 8/8 match at the HLA-A, HLA-B, HLA-C, and HLA-DRB1 loci to each other. In some embodiments, all the CD4⁺ T cells in the population have 2/2 match at the HLA-A locus to each other. In some embodiments, all the CD4⁺ T cells in the population have 2/2 match at the HLA-B locus to each other. In some embodiments, all the CD4⁺ T cells in the population have 2/2 match at the HLA-C locus to each other. In some embodiments, all the CD4⁺ T cells in the population have at least 3/4 or 4/4 match at the HLA-DRB1 and HLA DQB1 loci with each other. In some embodiments, all the CD4⁺ T cells in the population have an A*02 or A*24 allele.

In some embodiments, all of the CD4⁺ T cells in the population have less than 5/10, 6/10, 7/10, 8/10, or 9/10 match at the HLA-A, HLA-B, HLA-C, HLA-DRB1, and HLA-DQB1 loci to each other. In some embodiments, all of the CD4⁺ T cells in the population have less than 4/8, 5/8, 6/8, 7/8, or 8/8 match at the HLA-A, HLA-B, HLA-C, and HLA-DRB1 loci to each other. In some embodiments, all the CD4⁺ T cells in the population have less than 2/2 match at the HLA-A locus to each other. In some embodiments, all the CD4⁺ T cells in the population have less than 2/2 match at the HLA-B locus to each other. In some embodiments, all the CD4⁺ T cells in the population have less than 2/2 match at the HLA-C locus to each other. In some embodiments, all the CD4⁺ T cells in the population have less than 3/4 or 4/4 match at the HLA-DRB1 and HLA DQB1 loci with each other.

In some embodiments, all of the CD4⁺ T cells in the population have less than 5/10, 6/10, 7/10, 8/10, or 9/10 match at the HLA-A, HLA-B, HLA-C, HLA-DRB1, and HLA-DQB1 loci to each other. In some embodiments, all of the CD4⁺ T cells in the population have less than 4/8, 5/8, 6/8, 7/8, or 8/8 match at the HLA-A, HLA-B, HLA-C, and HLA-DRB1 loci to each other. In some embodiments, all the CD4⁺ T cells in the population have less than 2/2 match at the HLA-A locus to each other. In some embodiments, all the CD4⁺ T cells in the population have less than 2/2 match at the HLA-B locus to each other. In some embodiments, all the CD4⁺ T cells in the population have less than 2/2 match at the HLA-C locus to each other. In some embodiments, all the CD4⁺ T cells in the population have less than 3/4 or 4/4 match at the HLA-DRB1 and HLA DQB1 loci with each other.

In preferred embodiments, none of the at least two different T cell donors is a host to be treated with the CD4^{IL-10} cells. In preferred embodiments, none of the at least two different T cell donors is a donor of stem cells (e.g., HSC), tissue or organ that will be used together with the CD4^{IL-10} cells in the methods of treatment described herein.

In some embodiments, one or more of the T cell donors are HLA-mismatched or partially HLA-mismatched to the patient to be treated (host). In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the patient. In some embodiments, one or more of the T cell donors have less than 4/8, 5/8, 6/8, 7/8, or 8/8 match at the HLA-A, HLA-B, HLA-C, and HLA-DRB1 loci to the patient. In some embodiments, one or more of the T cell donors have less than 2/2 match at the HLA-A, HLA-B, or HLA-C locus to the patient. In some embodiments, one or more of the T cell donors have less than 2/4, 3/4 or 4/4 match at the HLA-DRB1 and HLA-DQB1 loci to the patient.

In some embodiments, one or more of the T cell donors are HLA-mismatched or partially HLA-mismatched with the HSC donor. In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the HSC donor. In some embodiments, one or more of the T cell donors have less than 4/8, 5/8, 6/8, 7/8, or 8/8 match at the HLA-A, HLA-B, HLA-C, and HLA-DRB1 loci to the HSC donor. In some embodiments, one or more of the T cell donors have less than 2/2 match at the HLA-A, HLA-B, or HLA-C locus to the HSC donor. In some embodiments, one or more of the T cell donors have less than 3/4 or 4/4 match at the HLA-DRB1 and HLA-DQB1 loci to the HSC donor.

In the preferred embodiments, none of the CD4⁺ T cells is immortalized.

### 6.3.2. Exogenous Polynucleotide Encoding IL-10

Polydonor CD4^{IL-10} cells of the present disclosure are CD4⁺ T cells that have been genetically modified to comprise an exogenous polynucleotide encoding IL-10. The exogenous polynucleotide comprises an IL-10-encoding polynucleotide segment operably linked to expression control elements.

The IL-10-encoding polynucleotide segment can encode IL-10 of a human, bonobo or rhesus. In some embodiments, the IL-10-encoding polynucleotide segment encodes human IL-10 having the sequence of SEQ ID NO: 1. In some embodiments, the IL-10-encoding polynucleotide segment encodes a variant of human IL-10 having at least 90%, 95%, 98%, or 99% sequence identity to SEQ ID NO: 1. In some embodiments, the IL-10-encoding polynucleotide segment has the nucleotide sequence of SEQ ID NO:2. In some embodiments, the IL-10-encoding polynucleotide segment has at least 90%, 95%, 98%, or 99% sequence identity to SEQ ID NO: 2.

In some embodiments, the IL-10-encoding polynucleotide segment encodes IL-10 of a Mus musculus, “MOUSE” (SEQ ID NO: 10); Rattus norvegicus, “RAT” (SEQ ID NO: 11); Macaca mulatta, “MACMU” (SEQ ID NO: 12); Gorilla gorilla, “GORILLA” (SEQ ID NO: 13); Macaca fascicularis, “CYNO” (SEQ ID NO: 14); Papio Anubis, “OLIVE BABOON” (SEQ ID NO: 15); Pan paniscus, “BONOBO” (SEQ ID NO: 16); Pan troglodytes, “CHIMP” (SEQ ID NO: 17); and EBVB9 (SEQ ID NO: 18). In some embodiments, the IL-10-encoding polynucleotide segment encodes a protein having at least 90%, 95%, 98%, or 99% sequence identity to IL-10 of a Mus musculus, “MOUSE” (SEQ ID NO: 10); Rattus norvegicus, “RAT” (SEQ ID NO: 11); Macaca mulatta, “MACMU” (SEQ ID NO: 12); Gorilla gorilla, “GORILLA” (SEQ ID NO: 13); Macaca fascicularis, “CYNO” (SEQ ID NO: 14); Papio Anubis, “OLIVE BABOON” (SEQ ID NO: 15); Pan paniscus, “BONOBO” (SEQ ID NO: 16); Pan troglodytes, “CHIMP” (SEQ ID NO: 17); and EBVB9 (SEQ ID NO: 18).

In some embodiments, the exogenous polynucleotide encodes viral-IL-10. In various embodiments, the exogenous polypeptide encodes IL-10 from HCMV, GMCMV, RhCMV, BaCMV, MOCMV, SMCMV, EBV, Bonobo-HV, BaLCV, OvHV-2, EHV-2, CyHV-3, AngHV-1, ORFV, BPSV, PCPV, LSDV, SPV, GPV, or CNPV. In some embodiments, the exogenous polypeptide encodes viral IL-10 from EBV or ORFV.

In some embodiments, the IL-10-encoding polynucleotide segment encodes a variant of human IL-10 having one, two, three, four, five, six, seven, eight, nine, ten or more amino acid substitutions compared to human IL-10 (e.g., SEQ ID NO: 1). In some embodiments, the one, two, three, four, five, six, seven, eight, nine or ten amino acid substitution are substitution(s) with amino acid(s) of viral IL-10 at corresponding amino acid position(s). In some embodiments, the IL-10-encoding polynucleotide segment encodes a variant of human IL-10 having one, two, three, four, five, six, seven, eight, nine, ten or more amino acid insertion, deletion or modification compared to human IL-10 (e.g, SEQ ID NO: 1). In some embodiments, the variant of human IL-10 has the sequence of SEQ ID NO: 8 or 9.

In some embodiments, the IL-10-encoding polynucleotide segment encodes a variant of human IL-10 having one, two, three, four, five, six, seven, eight, nine, ten or more amino acid substitutions, insertions, and/or deletions compared to human IL-10 (e.g., SEQ ID NO: 1). In some embodiments, the modifications are substitutions, insertions, and/or deletions with amino acids of Mus musculus, “MOUSE” (SEQ ID NO: 10); Rattus norvegicus, “RAT” (SEQ ID NO: 11); Macaca mulatta, “MACMU” (SEQ ID NO: 12); Gorilla gorilla, “GORILLA” (SEQ ID NO: 13); Macaca fascicularis, “CYNO” (SEQ ID NO: 14); Papio anubis, “OLIVE BABOON” (SEQ ID NO: 15); Pan paniscus, “BONOBO” (SEQ ID NO: 16); Pan troglodytes, “CHIMP” (SEQ ID NO: 17); and EBVB9 (SEQ ID NO: 18), at the corresponding positions. In some embodiments, the variant of human IL-10 has the sequence of SEQ ID NO: 19 or SEQ ID NO: 20.

In some embodiments, the IL-10-encoding polynucleotide segment encodes a variant of human IL-10 having reduced immunostimulatory activity compared to human IL-10. In some embodiments, the variant of human IL-10 includes I105A substitution. In some embodiments, a variant of human IL-10 is made using the method described in A Single Amino Acid Determines the Immunostimulatory Activity of Interleukin 10, J Exp Med, 191, 2, 2000, p. 213-223.

The exogenous polynucleotide further comprises expression control elements that direct expression of the encoded IL-10 in transduced CD4⁺ T cells.

In some embodiments, the expression control elements comprise a promoter capable of directing expression of IL-10 in CD4⁺ T cells. In some embodiments, the promoter drives constitutive expression of IL-10 in CD4⁺ T cells. In some embodiments, the promoter drives expression of IL-10 in activated CD4⁺ T cells.

In some embodiments, an inducible promoter is used to induce expression of IL-10 when therapeutically appropriate. In some embodiments, the IL-10 promoter is used. In some embodiments a tissue-specific promoter is used. In some embodiments, a lineage-specific promoter is used. In some embodiments, a ubiquitously expressed promoter is used.

In some embodiments, a native human promoter is used. In some embodiments, a human elongation factor (EF)1α promoter is used. In some embodiments, a human phosphoglycerate kinase promoter (PGK) is used. In some embodiments, a human ubiquitin C promoter (UBI-C) is used.

In some embodiments, a synthetic promoter is used. In certain embodiments, a minimal CMV core promoter is used. In particular embodiments, an inducible or constitutive bidirectional promoter is used. In specific embodiments, the synthetic bidirectional promoter disclosed in Amendola et al., Nature Biotechnology, 23(1):108-116 (2005) is used. This promoter can mediate coordinated transcription of two mRNAs in a ubiquitous or a tissue-specific manner. In certain embodiments, the bidirectional promoter induces expression of IL-10 and a selection marker.

In some embodiments, the exogenous polynucleotide further comprises a segment encoding a selection marker that permits selection of successfully transduced CD4⁺ T cells. In some embodiments, the selection marker is ΔNGFR. In certain embodiments, the selection marker is a polypeptide having the sequence of SEQ ID NO:3. In certain embodiments, the selection marker is a polypeptide having at least 90%, 95%, 98%, or 99% sequence identity to SEQ ID NO: 3. In particular embodiments, the nucleotide sequence encoding the ΔNGFR selection marker has the sequence of SEQ ID NO: 4. In some embodiments, the nucleotide sequence encoding the ΔNGFR selection marker has at least 90%, 95%, 98%, or 99% sequence identity to SEQ ID NO: 4.

In some embodiments, expression of the selection marker correlates with expression of IL-10 from the exogenous polynucleotide. In some embodiments, expression of the selection marker linearly correlates with expression of IL-10 from the exogenous polynucleotide. Accordingly, in some embodiments, expression of the selection marker is measured to infer expression of IL-10 from the exogenous polynucleotide.

In some embodiments, the selection marker is a truncated form of EGFR polypeptide. In some embodiments, the selection marker is a truncated form of the human EGFR polypeptide, optionally huEGFR disclosed in Wang et al. “A transgene-encoded cell surface polypeptide for selection, in vivo tracking, and ablation of engineered cells”, Blood, v. 118, n. 5 (2011), incorporated by reference in its entirety herein.

In some embodiments, the exogenous polynucleotide further comprises a sequence encoding an antibiotic resistance gene. In some embodiments, the exogenous polynucleotide comprises a sequence encoding an ampicillin resistance gene.

In typical embodiments, the exogenous polynucleotide is delivered into CD4+ T cells using a vector. In some embodiments, the vector is a plasmid vector. In some embodiments, the vector is a viral vector.

In certain embodiments, the exogenous polynucleotide is delivered into CD4+ T cells using a lentiviral vector and the exogenous polynucleotide comprises lentiviral vector sequences. In certain embodiments, a lentiviral vector disclosed in Mátrai et al., Molecular Therapy 18(3):477-490 (2010) (“Mátrai”), incorporated by reference herein, is used.

In some embodiments, the lentiviral vector is capable of integrating into the T cell nuclear genome. In some embodiments, the lentiviral vector is not capable of integrating into T cell nuclear genome. In some embodiments, an integration-deficient lentiviral vector is used. For example, in some embodiments, an integration-deficient or other lentiviral vector disclosed in Mátrai is used. In some embodiments, an integrase-defective lentivirus is used. For example, an integrase-defective lentivirus containing an inactivating mutation in the integrase (D64V) can be used as described in Mátrai et al., Hepatology 53:1696-1707 (2011), which is incorporated by reference herein, is used.

In some embodiments, the exogenous polynucleotide is integrated in the T cell nuclear genome. In some embodiments, the exogenous polynucleotide is not integrated in the nuclear genome. In some embodiments, the exogenous polynucleotide exists in the T cell cytoplasm.

In particular embodiments, the exogenous polynucleotide has the sequence of SEQ ID NO:5. In some embodiments, the exogenous polynucleotide has at least 90%, 95%, 98%, or 99% sequence identity to SEQ ID NO: 5.

### 6.3.3. Gene Expression of Polydonor CD4^{IL-10} T Cells

Polydonor CD4^{IL-10} T cells express IL-10. In some embodiments, polydonor CD4^{IL-10} T cells constitutively express IL-10. In some embodiments, polydonor CD4^{IL-10} T cells express IL-10 when activated.

In some embodiments, polydonor CD4^{IL-10} T cells constitutively express at least 100 μg of IL-10 per 10⁶ of the CD4⁺ T cells/mL of culture. In some embodiments, polydonor CD4^{IL-10} T cells constitutively express at least 200 pg, 500 pg, 1 ng, 5 ng, 10 ng, or 50 ng of IL-10 per 10⁶ of the CD4⁺ T cells/mL of culture.

In some embodiments, polydonor CD4^{IL-10} T cells express at least 1 ng or 2 ng IL-10 per 10⁶ of the CD4⁺ T cells/mL of culture after activation with a combination of anti-CD3 and anti-CD28 antibodies, or anti-CD3 antibody and anti-CD28 antibody coated beads. In some embodiments, polydonor CD4^{IL-10} T cells express at least 5 ng, 10 ng, 100 ng, 200 ng, or 500 ng IL-10 per 10⁶ of the CD4⁺ T cells/mL of culture after activation with anti-CD3 and anti-CD28 antibodies or CD3 antibody and CD28 antibody coated beads.

In various embodiments, the amount of IL-1β production is determined 12 hours, 24 hours, or 48 hours after activation using various methods for protein detection and measurement, such as ELISA, spectroscopic procedures, colorimetry, amino acid analysis, radiolabeling, Edman degradation, HPLC, western blotting, etc. In preferred embodiments, the amount of IL-1β production is determined by ELISA 48 hours after activation with anti-CD3 and anti-CD28 antibodies.

In some embodiments, polydonor CD4^{IL-10} T cells express IL-10 at a level at least 5-fold higher than unmodified CD4⁺ T cells. In some embodiments, polydonor CD4^{IL-10} T cells express IL-10 at a level at least 6, 7, 8, 9, 10, 11, 12, 13, 14, 15, 20, 30, 40, or 50-fold higher than unmodified CD4⁺ T cells.

In some embodiments, polydonor CD4^{IL-10} T cells further express a selection marker. In some embodiments, polydonor CD4^{IL-10} T cells express a protein typically expressed in Tr1 cells. In some embodiments, polydonor CD4^{IL-10} T cells express a marker protein characteristic of Tr1 cells.

In some embodiments, polydonor CD4^{IL-10} T cells express CD49b. In some embodiments, polydonor CD4^{IL-10} T cells express LAG-3. In some embodiments, polydonor CD4^{IL-10} T cells express TGF-β. In some embodiments, polydonor CD4^{IL-10} T cells express IFNγ. In some embodiments, polydonor CD4^{IL-10} T cells express granzyme B (GzB). In some embodiments, polydonor CD4^{IL-10} T cells release granzyme B (GzB) when activated with myeloid antigen-presenting cells or myeloid tumor cells In some embodiments, polydonor CD4^{IL-10} T cells express perforin. In some embodiments, polydonor CD4^{IL-10} T cells release perforin when activated with myeloid antigen-presenting cells or myeloid tumor cells In some embodiments, polydonor CD4^{IL-10} T cells express CD18. In some embodiments, polydonor CD4^{IL-10} T cells express CD2. In some embodiments, polydonor CD4^{IL-10} T cells express CD226. In some embodiments, polydonor CD4^{IL-10} T cells express IL-22. In some embodiments, polydonor CD4^{IL-10} T cells express IL-10.

In some embodiments, polydonor CD4^{IL-10} T cells exhibit at least one phenotypic function of Tr1 cells. In various embodiments, the function is secretion of IL-10, secretion of TGF-β, and by the specific killing of myeloid antigen-presenting cells through the release of Granzyme B (GzB) and perforin.

### 6.3.4. Product by Process

In typical embodiments, polydonor CD4^{IL-10} T cells are obtained by modifying CD4⁺ T cells with an exogenous polynucleotide encoding IL-10.

In some embodiments, the exogenous polynucleotide is introduced to CD4⁺ T cells by a viral vector or a plasmid vector. In particular embodiments, CD4⁺ T cells are transduced with a lentivirus containing a coding sequence of IL-10.

In some embodiments, polydonor CD4^{IL-10} T cells are generated by (i) pooling primary CD4⁺ T cells obtained from at least two different T cell donors; and (ii) modifying the pooled CD4⁺ T cells by introducing an exogenous polynucleotide encoding IL-10. In some embodiments, polydonor CD4^{IL-10} T cells are generated by (i) obtaining primary CD4⁺ T cells from at least two different T cell donors; (ii) separately modifying each donor's CD4⁺T cells by introducing an exogenous polynucleotide encoding IL-10, and then (iii) pooling the genetically modified CD4⁺ T cells.

In some embodiments, polydonor CD4^{IL-10} T cells have been cultured in the presence of proteins capable of activating CD4⁺ T cells. In some embodiments, polydonor CD4^{IL-10} T cells have been cultured in the presence of anti-CD3 antibody and anti-CD28 antibody, or anti-CD3 antibody and anti-CD28 antibody coated beads. In some embodiments, polydonor CD4^{IL-10} T cells have been cultured in the presence of anti-CD3 antibodies, anti-CD28 antibodies, and IL-2, or anti-CD3 antibody and anti-CD28 antibody coated beads and IL-2. In some embodiments, polydonor CD4^{IL-10} T cells have been cultured in the presence of T Cell TransAct™ from Miltenyi Biotec. In some embodiments, polydonor CD4^{IL-10} T cells have been cultured in the presence of ImmunoCult Human T Cell Activator™ from STEMCELL Technologies.

In some embodiments, polydonor CD4^{IL-10} T cells are in a frozen stock.

### 6.4. Pharmaceutical Compositions

In another aspect, pharmaceutical compositions are provided. The pharmaceutical comprises the polydonor CD4^{IL-10} T cells disclosed herein and a pharmaceutically acceptable carrier or diluent.

The pharmaceutical composition can be formulated for administration by any route of administration appropriate for human or veterinary medicine. In typical embodiments, the composition is formulated for intravenous (IV) administration. In some embodiments, the composition is formulated for intravenous (IV) infusion. In embodiments formulated for IV administration, the pharmaceutical composition will be in the form of a parenterally acceptable aqueous solution which is pyrogen-free and has suitable pH, isotonicity and stability.

In some embodiments, the pharmaceutically acceptable carrier or diluent is saline, lactated Ringer's solution, or other physiologically compatible solution. In various embodiments, the pharmaceutical composition solution comprises 2-20%, preferably 5%, human serum albumin.

In some embodiments, unit dosage forms of the pharmaceutical composition are provided that are adapted for administration of the pharmaceutical composition by systemic administration, in particular, for intravenous administration.

In some embodiments, the unit dosage form contains 10⁴ to 10¹¹ polydonor CD4^{IL-10} T cells, 10⁴ to 10¹⁰ polydonor CD4^{IL-10} T cells, 10⁴ to 10⁹ polydonor CD4^{IL-10} T cells, 10⁵ to 10¹⁰ polydonor CD4^{IL-10} T cells, 10⁵ to 10⁹ polydonor CD4^{IL-10} T cells, 10⁵ to 10⁸ polydonor CD4^{IL-10} T cells, or 10⁵ to 10⁷ polydonor CD4^{IL-10} T cells.

In typical embodiments, the pharmaceutical composition in the unit dosage form is in liquid form.

### 6.5. Methods of Making Polydonor CD4^{IL-10}° Cells

In another aspect, the present disclosure provides a method of making polydonor CD4^{IL-10} cells.

In some embodiments, the method comprises the steps of: (i) pooling primary CD4⁺ T cells obtained from at least two different T cell donors; and (ii) modifying the pooled CD4⁺ T cells by introducing an exogenous polynucleotide encoding IL-10. In other embodiments, the method comprises the steps of: (i) obtaining primary CD4⁺ T cells from at least two different T cell donors; (ii) separately modifying each donor's CD4⁺ T cells by introducing an exogenous polynucleotide encoding IL-10; and then (iii) pooling the genetically modified CD4⁺ T cells, thereby obtaining the polydonor CD4^{IL-10} cells. Various methods known in the art can be used to introduce an exogenous polynucleotide encoding IL-10 to primary CD4⁺ T cells.

In some embodiments, the method further comprises the step of incubating the primary CD4⁺ T cells or genetically-modified CD4⁺ T cells in the presence of an anti-CD3 antibody and anti-CD28 antibody, or anti-CD3 antibody and anti-CD28 antibody coated beads. In some embodiments, the method further comprises the step of incubating the primary CD4⁺ T cells or genetically-modified CD4⁺ T cells in the presence of anti-CD3 antibody, anti-CD28 antibody and IL-2 or anti-CD3 antibody and anti-CD28 antibody coated beads and IL-2. In some embodiments, the method further comprises the step of incubating the primary CD4⁺ T cells or genetically-modified CD4⁺ T cells in the presence of a mixture of feeder cells. In some embodiments, the method further comprises the step of incubating the primary CD4⁺ T cells or genetically-modified CD4⁺ T cells in the presence of nanopreparations of anti-CD3 antibody and anti-CD28 antibody. In some embodiments, the incubation is done in the presence of T Cell TransAct™ from Miltenyi Biotec. In some embodiments, the incubation is done in the presence of ImmunoCult Human T Cell Activator™ from STEMCELL Technologies.

In some embodiments, the incubation step is performed before introducing an exogenous polynucleotide encoding IL-10. In some embodiments, the incubation step is performed after (i) pooling primary CD4⁺ T cells obtained from at least two different T cell donors; but before (ii) modifying the pooled CD4⁺ T cells by introducing an exogenous polynucleotide encoding IL-10. In some embodiments, the incubation step is performed after (i) obtaining primary CD4⁺ T cells from at least two different T cell donors; but before (ii) separately modifying each donor's CD4⁺ T cells by introducing an exogenous polynucleotide encoding IL-10.

In some embodiments, the incubation step is performed after step (ii). In other words, in some embodiments, the incubation step is performed after (ii) modifying the pooled CD4⁺ T cells by introducing an exogenous polynucleotide encoding IL-10. In some embodiments, the incubation step is performed after (ii) separately modifying each donor's CD4⁺ T cells by introducing an exogenous polynucleotide encoding IL-10, but before (iii) pooling the genetically modified CD4⁺ T cells, thereby obtaining the genetically-modified CD4⁺ T cells. In some embodiments, the incubation step is performed after (iii) pooling the genetically modified CD4⁺ T cells, thereby obtaining the polydonor CD4^{IL-10} cells.

In some embodiments, the incubation step is performed more than once. In some embodiments, the incubation step is performed both before and after genetic modification of CD4⁺ T cells.

In some embodiments, the exogenous polynucleotide is introduced into the primary CD4⁺ T cells using a viral vector. In some embodiments, the viral vector is a lentiviral vector.

In some embodiments, the exogenous polynucleotide comprises a segment encoding IL-10 having the sequence of SEQ ID NO: 1. In some embodiments, the exogenous polynucleotide comprises a segment encoding IL-10 having at least 90%, 95%, 98%, or 99% sequence identity to SEQ ID NO: 1. In some embodiments, the IL-10-encoding polynucleotide segment has the sequence of SEQ ID NO: 2. In some embodiments, the IL-10-encoding polynucleotide segment has at least 90%, 95%, 98%, or 99% sequence identity to SEQ ID NO: 2. In some embodiments, the exogenous polynucleotide further comprises a segment encoding a marker permitting selection of successfully transduced CD4⁺ T cells. In some embodiments, the encoded selection marker is ΔNGFR. In certain embodiments, the encoded selection marker has the sequence of SEQ ID NO:3. In particular embodiments, the exogenous polynucleotide comprises a sequence of SEQ ID NO:4. In some embodiments, the encoded selection marker is a truncated form of human EGFR polypeptide.

In some embodiments, the method further comprises the step of isolating the genetically-modified CD4⁺ T cells expressing the selection marker, thereby generating an enriched population of genetically-modified CD4^{IL-10} cells.

In some embodiments, at least 70% of the genetically-modified CD4⁺ T cells in the enriched population express a selection marker. In some embodiments, at least 95% of the genetically-modified CD4⁺ T cells in the enriched population express a selection marker. In some embodiments, at least 96, 97, 98, or 99% of the genetically-modified CD4⁺ T cells in the enriched population express a selection marker.

In some embodiments, the method further comprises the step of incubating the enriched population of the genetically-modified CD4⁺ T cells. In some embodiments, the incubation is performed in the presence of anti-CD3 antibody and anti-CD28 antibody, or anti-CD3 antibody and anti-CD28 antibody coated beads. In some embodiments, the incubation is performed further in presence of IL-2. In some embodiments, the incubation is performed in the presence of feeder cells. In some embodiments, the incubation is performed in the presence of nanopreparations of anti-CD3 antibody and anti-CD28 antibody. In some embodiments, the incubation is performed in the presence of T Cell TransAct™ from Miltenyi Biotec. In some embodiments, the incubation is performed in the presence of ImmunoCult Human T Cell Activator™ from STEMCELL Technologies.

In some embodiments, the method further comprises the step of freezing the genetically-modified CD4⁺ T cells.

In some embodiments, the primary CD4⁺ T cells are from donors selected based on their HLA haplotypes. In some embodiments, the method further comprises the step of selecting T cell donors by analyzing their genetic information. In some embodiments, the method comprises the step of analyzing genetic information or HLA haplotype of potential T cell donors.

In some embodiments, the primary CD4⁺ T cells are from donors having at least a partial HLA match with a host to be treated with the primary CD4⁺ T cells or a modification thereof. In some embodiments, the primary CD4⁺ T cells are from donors having at least a partial HLA match with a stem cell (HSC), tissue or organ donor. In some embodiments, the primary CD4⁺ T cells are obtained from third party donors who are not biologically related with a host. In some embodiments, the primary CD4⁺ T cells are obtained from third party donors who are not biologically related with a stem cell, tissue or organ donor.

In some embodiments, in step (i), the primary CD4⁺ T cells are obtained from two, three, four, five, six, seven, eight, nine, or ten different T cell donors. In some embodiments, the at least two T cell donors have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other. In some embodiments, the at least two T cell donors have at least 1/8, 2/8, 3/8, 4/8, 5/8, 6/8, 7/8, or 8/8 match at the HLA-A, HLA-B, HLA-C, and HLA-DRB1 loci to each other. In some embodiments, the at least two T cell donors have 2/2 match at the HLA-A locus to each other. In some embodiments, the at least two T cell donors have 2/2 match at the HLA-B locus to each other. In some embodiments, the at least two T cell donors have 2/2 match at the HLA-C locus to each other. In some embodiments, the at least two T cell donors have at least 3/4 or 4/4 match at the HLA-DRB1 and HLA-DQB1 loci to each other. In some embodiments, each of the at least two T cell donors has an A*02 or A*24 allele.

In some embodiments, the at least two T cell donors have less than 5/10, 6/10, 7/10, 8/10, or 9/10 match at the HLA-A, HLA-B, HLA-C, HLA-DRB1, and HLA-DQB1 loci to each other. In some embodiments, the at least two T cell donors have less than 4/8, 5/8, 6/8, 7/8, or 8/8 match at the HLA-A, HLA-B, HLA-C, and HLA-DRB1 loci to each other. In some embodiments, the at least two T cell donors have less than 2/2 match at the HLA-A locus to each other. In some embodiments, the at least two T cell donors have less than 2/2 match at the HLA-B locus to each other. In some embodiments, the at least two T cell donors have less than 2/2 match at the HLA-C locus to each other. In some embodiments, the at least two T cell donors have less than 3/4 or 4/4 match at the HLA-DRB1 and HLA-DQB1 loci to each other.

In some embodiments, in step (i), the primary CD4⁺ T cells are obtained from one or more frozen stocks. In some embodiments, in step (i), the primary CD4⁺ T cells are obtained from unfrozen peripheral blood mononuclear cells of the at least two different T cell donors. In some embodiments, the method further comprises the step of isolating CD4⁺ T cells from the peripheral blood mononuclear cells. In some embodiments, in step (i), the primary CD4⁺ T cells are obtained from a liquid suspension. In some embodiments, the liquid suspension is obtained from a previously frozen stock.

In some embodiments, CD4⁺ T cells from donors are contacted with patient antigen-presenting cells (monocytes, dendritic cells, or DC-10 cells), generating allo-specific CD4⁺ T cells that are then modified to produce high levels of IL-10 (allo-CD4^{IL-10} cell).

In some embodiments, the method does not comprise the step of anergizing the CD4⁺ T cells in the presence of peripheral blood mononuclear cells (PBMCs) from a host. In some embodiments, the method does not comprise the step of anergizing the CD4⁺ T cells in the presence of recombinant IL-10 protein, wherein the recombinant IL-10 protein is not expressed from the CD4⁺ T cells. In some embodiments, the method does not comprise the step of anergizing the CD4⁺ T cells in the presence of DC10 cells from a host.

### 6.6. Methods of Using Polydonor CD4^{IL-10}° Cells

In yet another aspect, the present disclosure provides a method of treating a patient, comprising the step of administering the polydonor CD4^{IL-10} cells or the pharmaceutical composition provided herein to a patient in need of immune tolerization.

In some embodiments, the method further comprises the preceding step of thawing a frozen suspension of polydonor CD4^{IL-10} cells.

In some embodiments, the polydonor CD4^{IL-10} cells or the pharmaceutical composition prevents or reduces severity of pathogenic T cell response in the patient. In some embodiments, the polydonor CD4^{IL-10} cells or the pharmaceutical composition reduces inflammation. In some embodiments, the polydonor CD4^{IL-10} cells or the pharmaceutical composition enhances tissue repair. In some embodiments, the polydonor CD4^{IL-10} cells or the pharmaceutical composition enhances immunological tolerance to self and non-pathogenic antigens and maintain immune homeostasis. In some embodiments, the polydonor CD4^{IL-10} cells or the pharmaceutical composition downregulates pathogenic T-cell responses associated with organ transplantation, GvHD and various autoimmune and inflammatory diseases. In some embodiments, the polydonor CD4^{IL-10} cells or the pharmaceutical composition treats autoimmune disease. In some embodiments, the polydonor CD4^{IL-10} cells or the pharmaceutical composition reduces hyperactivity of NLPR3 inflammasome or reduces symptoms associated with hyperactivity of NLPR3 inflammasome. In some embodiments, the polydonor CD4^{IL-10} cells or the pharmaceutical composition induces death of tumor cells or reduces tumor growth. In some embodiments, the polydonor CD4^{IL-10} cells or the pharmaceutical composition increases disease free survival (e.g., absence of minimal residual disease). In some embodiments, the polydonor CD4^{IL-10} cells or the pharmaceutical composition induces wound healing or tissue repair.

In some embodiments, the polydonor CD4^{IL-10} cells or the pharmaceutical composition are administered at an amount effective to prevent or reduce severity of pathogenic T cell response in the patient. In some embodiments, the polydonor CD4^{IL-10} cells or the pharmaceutical composition are administered at an amount effective to reduce inflammation. In some embodiments, the polydonor CD4^{IL-10} cells or the pharmaceutical composition are administered at an amount effective to enhance tissue repair. In some embodiments, the polydonor CD4^{IL-10} cells or the pharmaceutical composition are administered at an amount effective to enhance immunological tolerance to self and pathogenic antigens and maintain immune homeostasis. In some embodiments, the polydonor CD4^{IL-10} cells or the pharmaceutical composition are administered at an amount effective to downregulate pathogenic T-cell responses associated with organ transplantation, GvHD and various autoimmune or inflammatory diseases. In some embodiments, the polydonor CD4^{IL-10} cells or the pharmaceutical composition are administered at an amount effective to treat autoimmune disease. In some embodiments, the polydonor CD4^{IL-10} cells or the pharmaceutical composition are administered at an amount effective to reduce hyperactivity of NLPR3 inflammasome or reduces symptoms associated with hyperactivity of NLPR3 inflammasome. In some embodiments, the polydonor CD4^{IL-10} cells or the pharmaceutical composition are administered at an amount effective to induce death of tumor cells or reduces tumor growth. In some embodiments, the polydonor CD4^{IL-10} cells or the pharmaceutical composition are administered at an amount effective to increase disease free survival (e.g., absence of minimal residual disease).

In some embodiments, the treatment method further comprises monitoring polydonor CD4^{IL-10} cells in a patient after administration. In some embodiments, the method comprises the step of detecting a selection marker in a biological sample obtained from the patient, thereby detecting presence or absence of polydonor CD4^{IL-10} T cells. In some embodiments, the selection marker is detected at multiple time points to trace changes in presence of polydonor CD4^{IL-10} cells in a patient. In some embodiments, the biological sample is a biopsy or blood sample from the patient.

The polydonor CD4^{IL-10} T cells are administered in a therapeutically effective amount. The amount can be determined based on the body weight and other clinical factors. In some embodiments, 10³ to 10⁹ cells/kg are administered. In some embodiments, 10³ to 10⁸ cells/kg are administered. In some embodiments, 10³ to 10⁷ cells/kg are administered. In some embodiments, 10³ to 10⁶ cells/kg are administered. In some embodiments, 10³ to 10⁵ cells/kg are administered. In some embodiments, 10³ to 10⁴ cells/kg are administered.

In various embodiments, polydonor CD4^{IL-10} T cells are administered on a therapeutically effective schedule. In some embodiments, polydonor CD4^{IL-10} T cells are administered once. In some embodiments, polydonor CD4^{IL-10} cells are administered every day, every 3 days, every 7 days, every 14 days, every 21 days, or every month.

The polydonor CD4^{IL-10} T cells can be administered according to different administration routes, such as systemically, subcutaneously, or intraperitoneally. In some embodiments, the cells are administered within a saline or physiological solution which may contain 2-20%, preferably 5% human serum albumin.

In some embodiments, administering the polydonor CD4^{IL-10} is prophylactic, in terms of completely or partially preventing a disease, condition, or symptoms thereof.

### 6.6.1. Methods of Reducing or Preventing GvHD

In some embodiments, the polydonor CD4^{IL-10} cells or the pharmaceutical composition comprising polydonor CD4^{IL-10} cells is used to treat a patient before a hematopoietic stem cell (HSC) transplant (HSCT), concurrently with an HSCT, or following an HSCT.

In various embodiments, the HSCT is a matched related HSCT. In various embodiments, the HSCT is a haploidentical HSCT, a mismatched related HSCT, or a mismatched unrelated HSCT.

In some embodiments, the patient has a hematological malignancy which requires treatment with allo-HSCT. In some embodiments, the hematological malignancy is mediated by aberrant myeloid cells.

In some embodiments, T cell donors are selected based on genetic information of a patient to be treated with polydonor CD4^{IL-10} cells and HSC, and/or genetic information of the HSC donor. In some embodiments, T cell donors are selected based on HLA haplotype of a patient to be treated with polydonor CD4^{IL-10} cells and HSC, and/or HLA haplotype of the HSC donor. In some embodiments, the method further comprises the step, prior to administering CD4^{IL-10} cells, of analyzing genetic information or HLA haplotype of T cell donors. In some embodiments, the method further comprises the step of analyzing genetic information or HLA haplotype of a host. In some embodiments, the method further comprises the step of analyzing genetic information or HLA haplotype of an HSC donor.

In some embodiments, T cell donors, a host and an HSC donor are not biologically related. In some embodiments, T cell donors, a host and an HSC donor have different HLA haplotypes. In some embodiments, T cell donors, a host and an HSC donor have at least partial mismatch in HLA haplotype. In some embodiments, T cell donors are selected when they have HLA haplotype with an HLA match over a threshold value.

In some embodiments, the HSC donor is partially HLA mismatched to the patient. In some embodiments, the HSC donor has less than 5/10, 6/10, 7/10, 8/10, 9/10 or 10/10 match at the HLA-A, HLA-B, HLA-C, HLA-DRB1, and HLA-DQB1 loci to the patient. In some embodiments, the HSC donor has less than 4/8, 5/8, 6/8, 7/8, or 8/8 match at the HLA-A, HLA-B, HLA-C, and HLA-DRB1 loci to the patient. In some embodiments, the HSC donor has less than 2/2 match at the HLA-A, HLA-B, or HLA-C locus to the patient. In some embodiments, the HSC donor has less than 3/4 or 4/4 match at the HLA-DRB1 and HLA-DQB1 loci to the patient.

In some embodiments, one or more of the T cell donors are HLA-mismatched or partially HLA-mismatched to the patient. In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the patient. In some embodiments, one or more of the T cell donors have less than 4/8, 5/8, 6/8, 7/8, or 8/8 match at the HLA-A, HLA-B, HLA-C, and HLA-DRB1 loci to the patient. In some embodiments, one or more of the T cell donors have less than 2/2 match at the HLA-A, HLA-B, or HLA-C locus to the patient. In some embodiments, one or more of the T cell donors have less than 2/4, 3/4 or 4/4 match at the HLA-DRB1 and HLA-DQB1 loci to the patient.

In some embodiments, one or more of the T cell donors are HLA-mismatched or partially HLA-mismatched with the HSC donor. In some embodiments, one or more of the T cell donors have less than 5/10, 6/10, 7/10, 8/10, 9/10 or 10/10 match at the HLA-A, HLA-B, HLA-C, HLA-DRB1, and HLA-DQB1 loci to the HSC donor. In some embodiments, one or more of the T cell donors have less than 4/8, 5/8, 6/8, 7/8, or 8/8 match at the HLA-A, HLA-B, HLA-C, and HLA-DRB1 loci to the HSC donor. In some embodiments, one or more of the T cell donors have less than 2/2 match at the HLA-A, HLA-B, or HLA-C locus to the HSC donor. In some embodiments, one or more of the T cell donors have less than 3/4 or 4/4 match at the HLA-DRB1 and HLA-DQB1 loci to the HSC donor. In some embodiments, the polydonor CD4^{IL-10} cells or the pharmaceutical composition prevents or reduces severity of GvHD by the transplanted hematopoietic stem cells.

In some embodiments, the polydonor CD4^{IL-10} cells or the pharmaceutical composition prevents or reduces severity of pathological T cell response by the transplanted hematopoietic cells. In specific embodiments, the polydonor CD4^{IL-10} cells prevents or reduces GvHD.

In some embodiments, the polydonor CD4^{IL-10} cells or the pharmaceutical composition prevents or reduces severity of tissue damage induced by the pathogenic T cells or the inflammation.

### 6.6.2. Methods of Treating Cancer

In some embodiments, polydonor CD4^{IL-10} cells are used for treatment of cancer. In preferred embodiments, the polydonor CD4^{IL-10} cells directly mediate anti-tumor effects and in particular embodiments, an anti-leukemic effect.

In some embodiments, polydonor CD4^{IL-10} cells are administered in combination with allogeneic mononuclear cells or PBMC for treatment of cancer. In some embodiments, polydonor CD4^{IL-10} cells are administered prior to or subsequence to administration of PBMC. In some embodiments, polydonor CD4^{IL-10} cells and allogeneic mononuclear cells or PBMC are administered concurrently.

In some embodiments, polydonor CD4^{IL-10} cells and allogeneic mononuclear cells or PBMC are administered at 1:3, 1:2, 1:1, 2:1 or 3:1 ratio.

In some embodiments, the neoplastic cells express CD13. In some embodiments, the neoplastic cells express HLA-class I. In some embodiments, the neoplastic cells express CD54. In some embodiments, the neoplastic cells express CD13, HLA-class I and CD54. In some embodiments, the neoplastic cells express CD112. In some embodiments, the neoplastic cells express CD58. In some embodiments, the neoplastic cells express CD155. In some embodiments, the tumor expresses CD112, CD58, or CD155. In various embodiments, the tumor is a solid or hematological tumor.

In some embodiments, the patient has a cancer selected from the group consisting of: Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Acute Lymphocytic (ALL), Acute Myeloid (AML, including myeloid sarcoma and leukemia cutis), Chronic Lymphocytic (CLL), Chronic Myeloid (CML) Leukemia, Chronic Myelomonocytic (CMML), Leukemia in Children, Liver Cancer, Lung Cancer, Lung Cancer with Non-Small Cell, Lung Cancer with Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

In some embodiments, the cancer is a myeloid tumor. In particular embodiments, the cancer is AML or CML. In some embodiments, the cancer is a myeloid tumor.

In some embodiments, the method is used to treat a hematological cancer affecting blood, bone marrow, and lymph nodes. In various embodiments, the hematological cancer is a lymphoma (e.g. Hodgkin's Lymphoma), lymphocytic leukemias, myeloma. In various embodiments, the hematological cancer is acute or chronic myelogenous (myeloid) leukemia (AML, CML), or a myelodysplastic syndrome.

In some embodiments, the cancer is refractory or resistant to a therapeutic intervention.

In some embodiments, the polydonor CD4^{IL-10} cells are used in combination with a therapeutic intervention. The combination may be simultaneous or performed at different times. Preferably the therapeutic intervention is selected from the group consisting of: chemotherapy, radiotherapy, allo-HSCT, immune suppression, blood transfusion, bone marrow transplant, growth factors, biologicals.

In some embodiments, the polydonor CD4^{IL-10} cells induce cell death of tumor infiltrating and tumor growth promoting myeloid lineage cells (e.g., monocytes, macrophages, neutrophils).

### 6.6.3. Methods of Treating Inflammatory or Autoimmune Disease

In some embodiments, polydonor CD4^{IL-10} cells are administered to treat inflammatory or autoimmune disease. In some embodiments, polydonor CD^{4IL-10} cells are administered to treat a disease or disorder involving hyperactivity of NLPR3 inflammasome.

The NOD-like receptor family (NLR) protein NLRP3 is an intracellular signaling molecule that senses danger signals from pathogenic, environmental or endogenous source. Following activation, NLPR3 interacts with caspase-1, forming a complex termed the inflammasome. This results in the activation of caspase-1, which cleaves the pro-inflammatory cytokines IL-1β and IL-18 to their active forms and mediates a type of inflammatory cell death known as pyroptosis.

In some embodiments, polydonor CD4^{IL-10} cells are administered to treat an inflammatory disease selected from Muckle-Wells syndrome (MWS), familial cold auto-inflammatory syndrome (FCAS) and neonatal onset multi-system inflammatory disease (NOMID). In some embodiments, polydonor CD4^{IL-10} cells are administered to treat a chronic disease selected from metabolic syndrome, type 2 diabetes, atherosclerosis, Alzheimer, Parkinson, ALS, non-alcoholic steatohepatitis, osteoarthritis, silicosis, asbestosis, gout, and lung fibrosis. In some embodiments, polydonor CD4^{IL-10} cells are administered to treat Crohn's disease, Ulcerative colitis, Multiple sclerosis and systemic lupus erythromytosis or inflammatory eye diseases such as diabetic retinopathy, acute glaucoma and age related macular degeneration.

In some embodiments, polydonor CD4^{IL-10} cells are administered to treat a disease associated with NLRP3. The disease can be selected from the group consisting of: CAPS, NASH, Alzheimer, Parkinson, cardiovascular disease, osteoarthritis, gout, pseudogout, nephrocalcinosis, type II diabetes, Sjogren syndrome, sickle cell disease (SCD), AMD, infections, cerebral malaria, asbestosis, contact hypersensitivity, sunburn, silicosis, cystic fibrosis, inflammatory bowel disease, nephrocalcitosis, ALS, myelodysplastic syndrome, and uveitis.

In some embodiments, the disease is a brain disorder selected from Parkinson, Alzheimer, age-related cognitive impairment, frontotemporal dementia, traumatic brain injury, intracerebral hemorrhage, sepsis-associated encephalopathy, cerebral ischemia, subarachnoid hemorrhage, epilepsy, acrylamide poisoning, opioid-induced neuroinflammation, chronic migraine, perioperative neurocognitive disorder, poststroke cognitive impairment, post-cardiac arrest cognitive impairment, social isolation-induced cognitive impairment, anxiety and post-traumatic stress disorder.

In some embodiments, the disease is a lung disorder selected from asthma, IR lung injury, ARDS/COPD, particulate matter-induced lung injury, radiation pneumonitis, pulmonary hypertension, sarcoidosis, cystic fibrosis, and allergic rhinitis.

In some embodiments, the disease is a heart disorder selected from atherosclerosis, heart failure, hypertension, myocardial infarction, atrial fibrillation, cardiac injury induced by metabolic dysfunction, and endothelial dysfunction.

In some embodiments, the disease is a gastrointestinal disease, such as colitis. In some embodiments, the disease is a liver disorder selected from acute liver failure, circadian regulation of immunity, NASH, cognitive dysfunction in diabetes, IR liver injury, idiosyncratic drug-induced liver injury and liver fibrosis. In some embodiments, the disease is a pancreas or kidney disorder selected from diabetic encephalopathy, diabetes-associated atherosclerosis, insulin resistance, islet transplantation rejection, chronic crystal nephropathy, renal fibrosis, I/R kidney injury, obesity-associated renal disease, and renal hypertension. In some embodiments, the disease is a skin or eye disorder selected from psoriasis and retinal neovascularization. In some embodiments, the disease is a reproductive disorder such as preterm birth. In some embodiments, the disease is an immune disorder selected from primary dysmenorrhea, innate immunity, innate to adaptive immunity, systemic lupus erythematosus-lupus nephritis, and multiple sclerosis. In some embodiments, the disease is an inheritable disorder selected from Muckle-Wells syndrome, rheumatoid arthritis, sickle cell disease and VCP-associated disease. In some embodiments, the disease is a pain disorder selected from multiple sclerosis-associated neuropathic pain, chronic prostatitis/chronic pelvic pain, cancer-induced bone pain, and hyperalgesia. In some embodiments, the disease is cancer, such as human squamous cell carcinoma of head and neck cancer. In some embodiments, the disease is an infective disorder, such as bacterial, viral or parasitic infection.

In some embodiments, polydonor CD4^{IL-10} cells are used in combination with a currently available treatments for NLRP3 related diseases, such as a biologic agent that target IL-1. The biologic agent includes the recombinant IL-1 receptor antagonist Anakinra, the neutralizing IL-1β antibody Canakinumab and the soluble decoy IL-1 receptor Rilonacept.

In some embodiments, polydonor CD4^{IL-10} cells are administered to treat a disease selected from Type 2 diabetes, metabolic syndrome, cardiovascular diseases, SLE, MS, CD, Ulcerative colitis (UC), osteoarthritis, Nonalcoholic steatohepatitis (Nash), Parkinson, ALS, lung fibrosis, silicosis, asbestosis, diabetic retinopathy, and age-related macular degeneration.

In some embodiments, polydonor CD^{4IL-10} cells are administered to treat inflammation. The inflammation can be related to coronary artery disease (CAD), Type 2 diabetes, neurodegenerative diseases, or inflammatory bowel disease, but is not limited thereto.

In some embodiments, polydonor CD^{4IL-10} cells are administered to treat a disease or disorder involving increased IL-1β production by activated monocytes, macrophages or dendritic cells. In some embodiments, polydonor CD^{4IL-10} cells are administered to treat a disease or disorder involving increased IL-18 production by activated monocytes, macrophages or dendritic cells. In some embodiments, polydonor CD^{4IL-10} cells are administered to treat a disease or disorder involving increased mature caspase 1 production by activated monocytes, macrophages or dendritic cells.

In some embodiments, polydonor CD^{4IL-10} cells are administered to reduce IL-1 production by activated monocytes, macrophages or dendritic cells. In some embodiments, polydonor CD^{4IL-10} cells are administered to reduce IL-18 production by activated monocytes, macrophages or dendritic cells. In some embodiments, polydonor CD^{4IL-10} cells are administered to reduce mature caspase 1 production by activated monocytes, macrophages or dendritic cells.

### 6.6.4. Methods of Treating Other Disorders

In some embodiments, polydonor CD4^{IL-10} cells are administered to treat autoimmune disease.

In some embodiments, the autoimmune disease is selected from the group consisting of: type-1 diabetes, autoimmune uveitis, autoimmune hepatitis, vitiligo, alopecia areata, rheumatoid arthritis, psoriasis, psoriatic arthritis, multiple sclerosis, systemic lupus, inflammatory bowel disease, Addison's disease, Graves' disease, Sjögren's syndrome, Hashimoto's thyroiditis, myasthenia gravis, autoimmune vasculitis, pernicious anemia, ulcerative colitis, bullous diseases, scleroderma, and celiac disease. In some embodiments, the autoimmune disease is Crohn's disease, ulcerative colitis, celiac disease, type-1 diabetes, lupus, psoriasis, psoriatic arthritis, or rheumatoid arthritis. In some embodiments, the patient has an allergic or atopic disease. The allergic or atopic disease can be selected from the group consisting of: asthma, atopic dermatitis, and rhinitis. In some embodiments, the patient has a food allergy.

In some embodiments, polydonor CD4^{IL-10} cells are administered to prevent or reduce severity of pathogenic T cell response to cell and organ transplantation other than HSCT. In some embodiments, the method comprises the step of organ transplantation to the patient, either prior to or subsequent to administration of polydonor CD4^{IL-10} T cells or the pharmaceutical composition. In certain embodiments, the organ is a kidney, a heart, or pancreatic islet cells. In preferred embodiments, the polydonor CD4^{IL-10} cells or the pharmaceutical composition prevents or reduces severity of host rejection of the organ transplantation.

In some embodiments, polydonor CD4^{IL-10} cells are administered to prevent or reduce immune response associated with gene therapy, e.g., administration of recombinant AAV (rAAV). In these embodiments, the method further comprises the step of administering a recombinant AAV to the patient, either prior to or subsequent to administration of the polydonor CD4^{IL-10} cells or the pharmaceutical composition.

In some embodiments, polydonor CD4^{IL-10} cells are administered to prevent or reduce immune response associated with transplantation of iPS-derived tissues or cells. The iPS-derived tissues and cells include, but are not limited to cardiomyocytes, hepatocytes, epithelial cells, cartilage, bone and muscle cells, neurons.

In some embodiments, polydonor CD4^{IL-10} cells are administered to reduce patient hyperactive immune response to viral infection. In some embodiments, the virus is SARS-coV-2. In some embodiments, polydonor CD4^{IL-10} cells are administered to reduce hyperactive immune responses to bacterial infections, such as toxic shock and cytokine storm.

In some embodiments, the method further comprises the step of administering an immunogenic therapeutic protein to the patient, either prior to or subsequent to administration of the population of polydonor CD4^{IL-10} cells or the pharmaceutical composition. In some embodiments, the population of polydonor CD4^{IL-10} cells, or the pharmaceutical composition reduces immune responses against the immunogenic therapeutic protein. In some embodiments, the immunogenic therapeutic protein is selected from a therapeutic antibody, a factor VIII replacement, a cytokine, and a cytokine mutein.

### 6.7. EXAMPLES

The following examples are provided by way of illustration not limitation.

### 6.7.1. Summary of Experimental Observations

The present disclosure provides the methods for production and use of highly purified, allogeneic CD4⁺ T cells that have been transduced with a bidirectional lentiviral vector containing the human IL-10 gene and a truncated, non-signaling form of the human NGFR. The successfully transduced CD4⁺ T cells were purified utilizing a NGFR specific monoclonal antibody resulting in >95% pure IL-10 producing and NGFR expressing CD4⁺ T cells (designated CD4^{IL-10} cells). CD4^{IL-10} cells from 3 different allogeneic HLA mismatched donors were pooled at 1:1:1 ratios.

These pooled populations, also referred to herein as polydonor CD4^{IL-10} cells, had cytokine production profiles comparable to those of single-donor CD4^{IL-10} cells and naturally derived type 1 regulatory T (Tr1) cells. They produce high levels of IL-10 and IL-22, variable levels of IFN-γ and IL-5 and low levels of IL-4. The polydonor CD4^{IL-10} cells were polyclonal (has multiple antigen specificities) and suppressed proliferation of both allogeneic CD4⁺ and CD8⁺ T cells in vitro. In addition, they specifically killed myeloid leukemia cells in vitro. Additionally, the polydonor CD4^{IL-10} cells inhibited NLPR3 inflammasome activation and the pro inflammatory IL-1β and IL-18 production by human monocytes in vitro.

Adoptive transfer of polydonor CD4^{IL-10} cells in a humanized mouse model for Graft versus Host Disease (GvHD) indicated that these cells efficiently home to the spleen and bone marrow Adoptive transfer of polydonor CD4^{IL-10} cells in a humanized mouse model of GvHD inhibited severe xeno-GvHD induced by human CD4⁺ T cells or PBMC Importantly, even at high concentrations, polydonor CD4^{IL-10} cells did not induce GvHD by themselves. Additionally, polydonor CD4^{IL-10} cells had cytotoxic effects on cancer cells in an NSG mouse intravenously injected with ALL-CM cells. Injection of single- and polydonor CD4^{IL-10} cells 3 days after administration of the ALL-CM cells (when already massive expansion of these cells is ongoing) resulted in inhibition of tumor growth. These results indicate that polydonor CD4^{IL-10} cells have direct therapeutic anti myeloid leukemia effects in vivo. When the single- and polydonor CD4^{IL-10} cells were administered with PBMC, the CD4^{IL-10} cells further down regulated xeno-GvHD induced by allogeneic PBMC.

These results demonstrate that polydonor CD4^{IL-10} cells can be used for the treatment and/or prevention of GvHD; can be used as an adjunct to allogeneic hematopoietic stem cell transplant (HSCT) for treatment of leukemias and other malignancies to reduce GvHD while preserving GvL or GvT therapeutic effects of the HSCT; and for treating cell and organ rejection and autoimmune and inflammatory diseases.

### 6.7.2. Example 1: Generation of Polydonor CD4^{IL-10} Cells

### Vector Production

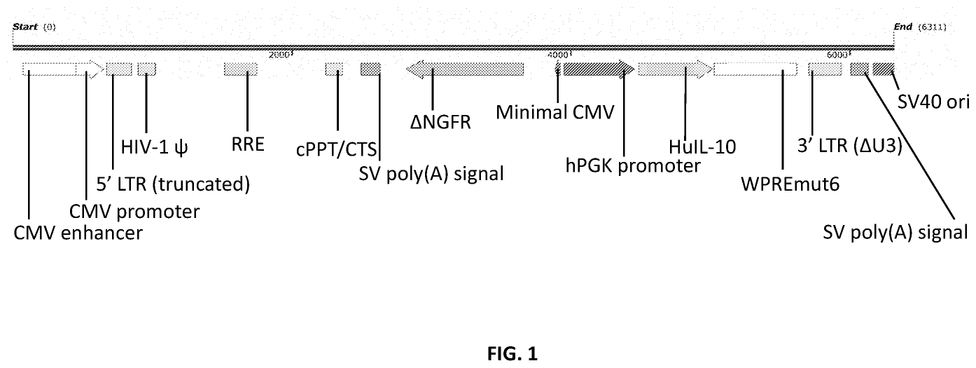

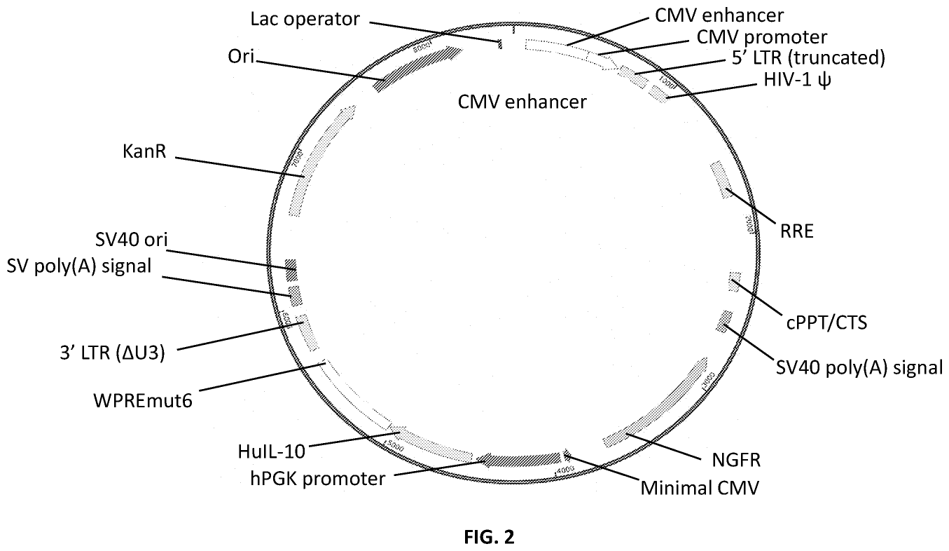

Polydonor CD4^{IL-10} cells were produced by transduction with a lentiviral vector (LV-IL-10/ΔNGFR) containing coding sequences of both the human IL-10 and a truncated form of the NGFR (ΔNGFR) (FIGS. 1 and 2), as described in WO2016/146542, incorporated by reference in its entirety herein. The sequence of the plasmid encoding for human IL-10 and ΔNGFR (pLVIL-10) used to manufacture LV-IL-10/ΔNGFR is provided as SEQ ID NO:5. In short, pLVIL-10 was generating by ligating the coding sequence of human IL-10 from 549 bp fragment of pH15C (ATCC 68192)) into plasmid #1074.1071.hPGK.GFP.WPRE.mhCMV.dNGFR.SV40PA. The presence of the bidirectional promoter (human PGK promoter plus minimal core element of the CMV promoter in the opposite direction) allows co-expression of the two transgenes. The plasmid further contains a coding sequence of an antibiotic resistance gene (e.g., ampicillin or kanamycin).

The lentiviral vectors were produced by Ca₃PO₄ transient four-plasmid co-transfection into 293T cells and concentrated by ultracentrifugation: 1 μM sodium butyrate was added to the cultures for vector collection. Titer was estimated on 293T cells by limiting dilution, and vector particles were measured by HIV-1 Gag p24 antigen immune capture (NEN Life Science Products; Waltham, MA). Vector infectivity was calculated as the ratio between titer and particle. For concentrated vectors, titers ranged from 5×10⁸ to 6×10⁹ transducing units/mL, and infectivity from 5×10⁴ to 5×10⁵ transducing units/ng.

### Production of CD4^{IL-10} Cells

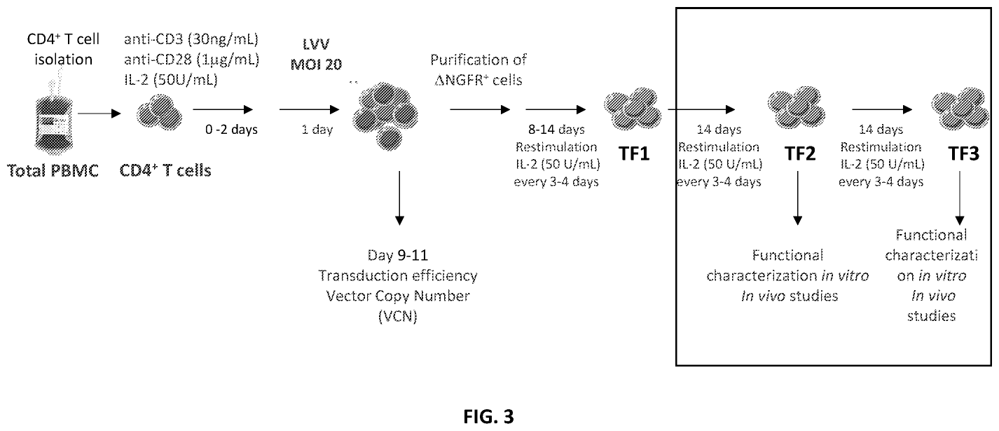

FIG. 3 is a schematic representation of the production process of CD4^{IL-10} cells. CD4⁺ T cells from healthy donors were purified. Human CD4⁺ T cells were activated with soluble anti-CD3, soluble anti-CD28 mAbs, and rhIL-2 (50 U/mL) for up to 48 hours before transduction with a bidirectional lentiviral vector encoding for human IL-10 and a truncated form the human NGF receptor (LV-IL-10/ΔNGFR) at multiplicity of infection (MOI) of 20.

After 9-11 days, transduced cells were analyzed by FACS for the expression of ΔNGFR, and the vector copy number (VCN) was quantified by digital droplet PCR (ddPCR).

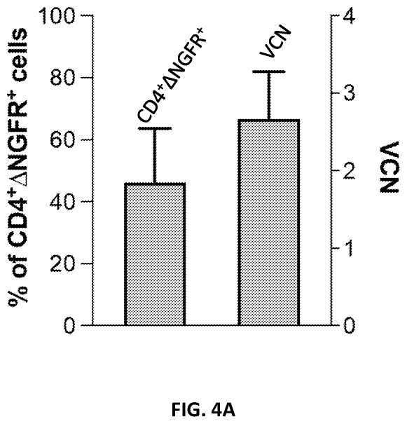

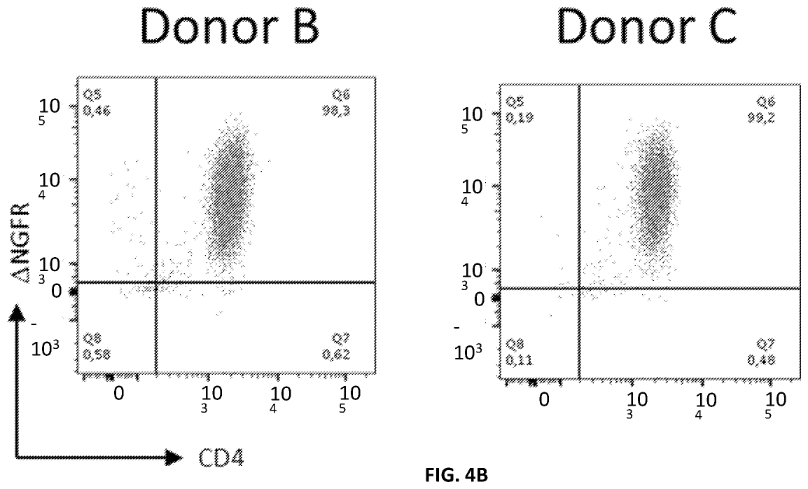

The mean transduction efficiency of CD4⁺ T cells from 10 different donors was 45±17% with VCN of 2.7±0.6%. FIG. 4A shows percentages of CD4⁺ΔNGFR⁺ cells (mean±SD, n=10 left bar) and vector copy numbers (VCN, mean±SD, n=10 right bar) in human CD4⁺ T cells transduced with LV-IL-10/ΔNGFR (a bidirectional lentiviral vector encoding for human IL-10 and a truncated form the human NGF receptor). The frequency of CD4⁺ΔNGFR⁺ cells and the vector copy numbers were quantified by digital droplet PCR (ddPCR) in CD4^{IL-10} cells.

ΔNGFR⁺ T cells were purified using anti-CD271 mAb-coated microbeads and resulted in >95% pure CD4^{IL-10} cells populations. After purification, cells were stained with markers for CD4 and ΔNGFR and analyzed by FACS. The data showed purity resulting from the purification step was over 98%. FIG. 4B shows FACS data from two representative donors (Donor B and Donor C) out of 10 donors tested. The purity of the CD4^{IL-10} cells for these two donors was respectively 98.3% and 99.2%. The purified CD4^{IL-10} cells were restimulated 3 times at 14 day intervals and their in vitro and in vivo functions were tested after the second (TF2) and or third restimulation (TF3) functions.

Resting CD4^{IL-10} cells produced IL-10 constitutively. Upon activation, the level of IL-10 produced was strongly enhanced.

CD4^{IL-10}° Cells have a Cytokine Production Profile which is Comparable to that of Naturally Derived Tr1 Cells.

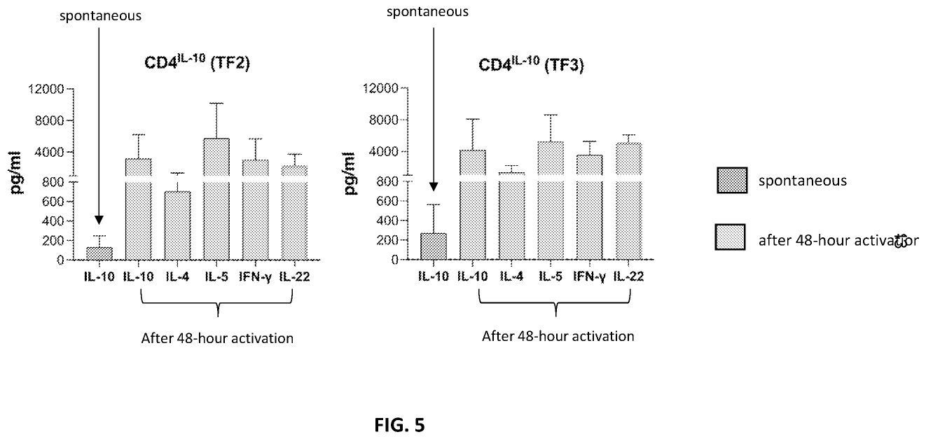

Cytokine production profiles of single donor CD4^{IL-10} cells were analyzed after the second (TF2) and third (TF3) restimulation and the results are provided in FIG. 5. Specifically, CD4^{IL-10} cells (2×10⁵ cells in 200 μl) were restimulated as previously described (Andolfi et al. Mol Ther. 2012; 20(9):1778-1790 and Locafaro et al. Mol Ther. 2017; 25(10):2254-2269). At day 14, after the 2^{nd} round (TF2) and 3^{rd} round (TF3) of restimulation, CD4^{IL-10} cells were left unstimulated or were activated with with immobilized CD3 (10 μg/mL) and soluble CD28 mAb (1 μg/mL) for 48 hours. Culture supernatants were collected and levels of IL-10, IL-4, IL-5, IFN-γ and IL-22 were determined by ELISA. All samples were tested in triplicate. Mean±SD, n=8 donors tested are presented. The results provided in FIG. 5 show that CD4^{IL-10} cells stimulated with immobilized anti-CD3 and soluble anti-CD28 mAbs show a Tr1 cell cytokine production profile.

Although variations between the different donors were observed, the overall cytokine production profiles after the second (TF2) (FIG. 5 left panel) or the third (TF3) (FIG. 5 right panel) restimulation were comparable and reflected those of Tr1 cells (Roncarolo et al., Immunity, 2018). Like Tr1 cells, the CD4^{IL-10} cells produced high levels of IL-10 and IL-22, variable levels of IL-5 and IFN-γ, but relatively low levels of IL-4 and undetectable levels of IL-2 (not shown).

### CD4^{IL-10} Cells Express High Levels of Granzyme B and Selectively Kill Myeloid Leukemia Cells

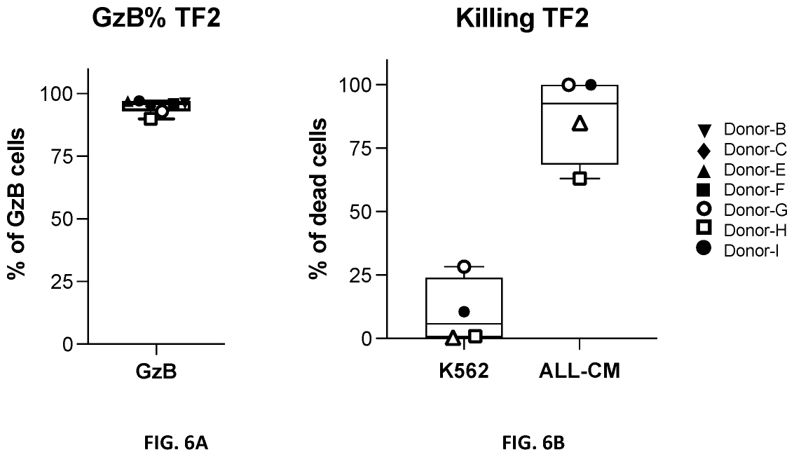

The CD4^{IL-10} cells were further analyzed after the 2^{nd} round (TF2) of restimulation for expression of granzyme B (GzB). The data in FIG. 6A show that more than 95% of all CD4^{IL-10} cells derived from 7 different donors expressed high levels of Granzyme B.

The CD4^{IL-10} cells from the 2^{nd} round (TF2) of restimulation were further analyzed for their cytotoxic effects against a human myeloid leukemia cell line (ALL-CM) and an erythroid leukemia cell line (K562). CD4^{IL-10} cells (10⁵/well) were co-cultured with K562 and ALL-CM cells (10⁵/well) at 1:1 ratio for 3 days. Residual leukemic cell lines (CD45^{low}, CD3−) were counted by FACS for each target cell.

The CD4^{IL-10} cells selectively killed the myeloid leukemia cells (ALL-CM) as shown in FIG. 6B. The % of killed ALL-CM cells varied between 62% and 100%, whereas the killing of the erythroid leukemia cell line K562 (which are highly sensitive for nonspecific cytotoxic and natural killer (NK) cell activities) varied between 0 and 27% (4 different donors tested). Taken together, these data confirm that CD4^{IL-10} cells express Granzyme B and efficiently kill myeloid leukemia cells. As expected, some variations in the killing capacity of the CD4^{IL-10} cells derived from individual donors was observed.

CD4^{IL-10} Cells Suppress the Proliferative Responses of Both Allogeneic CD4⁺ and CD8⁺ T Cells

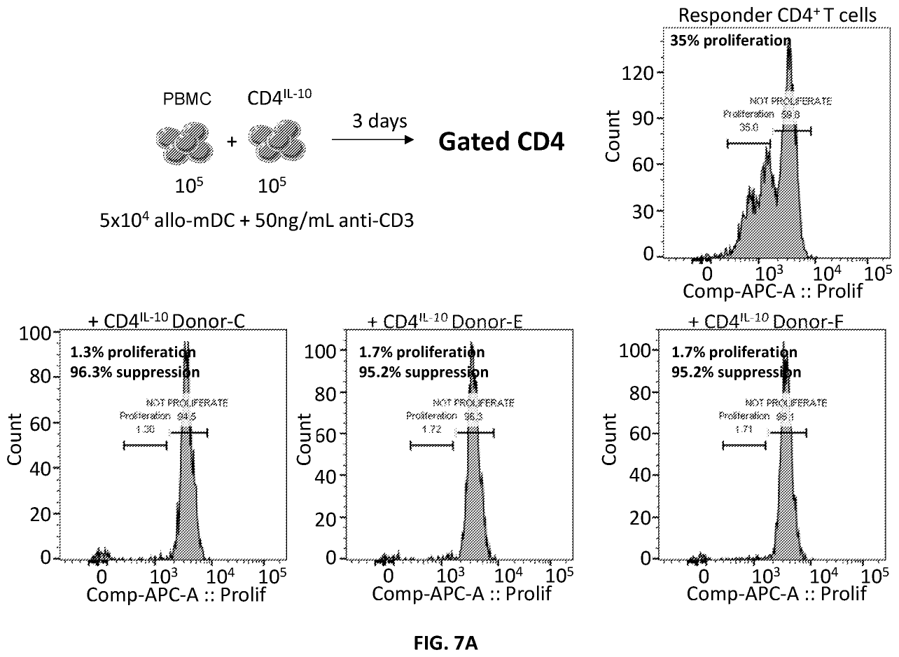

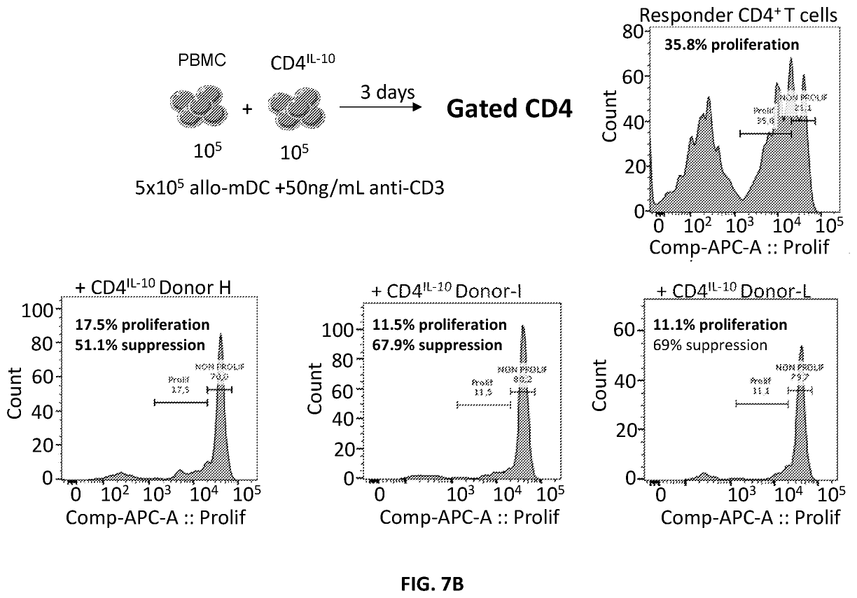

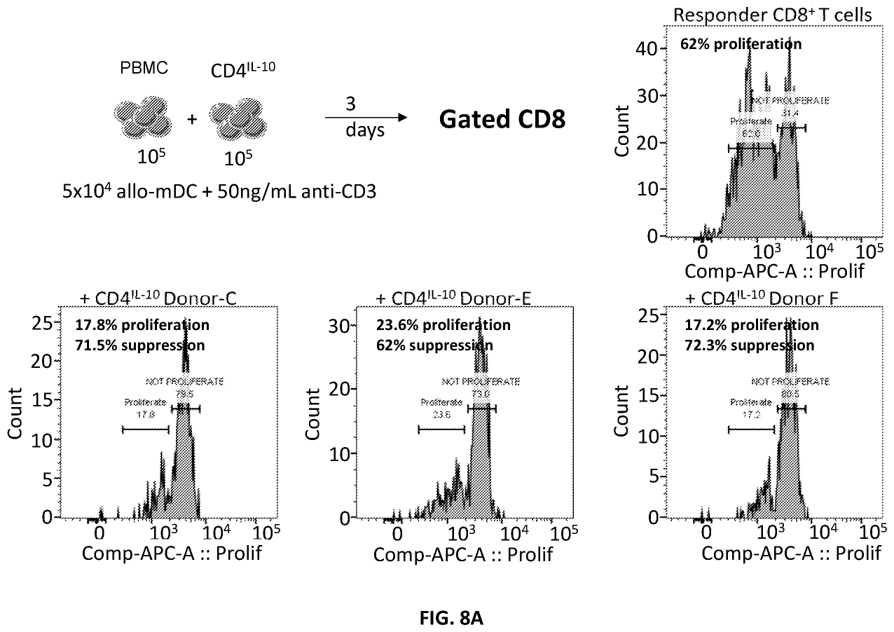

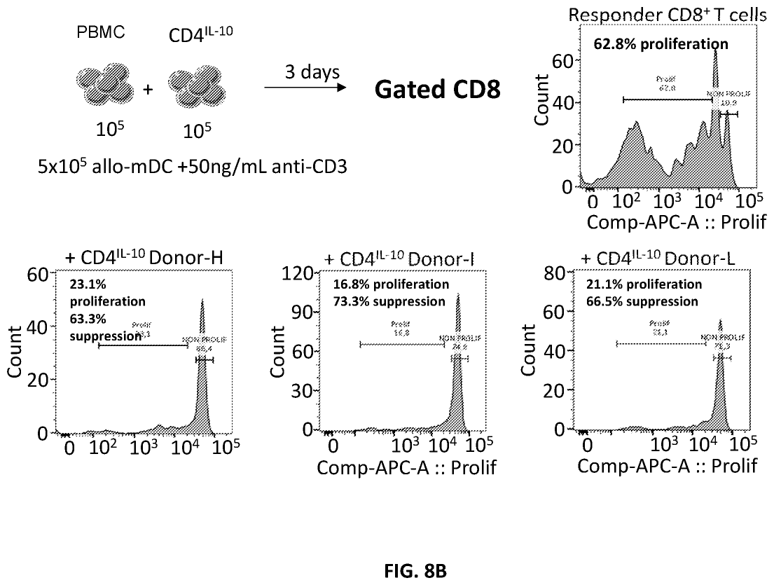

The CD4^{IL-10} cells were also analyzed for their effects on allogeneic CD4⁺ T cells or CD8⁺ T cells. Specifically, allogeneic PBMC cells were labeled with eFluor® 670 (5×10⁴ cells/well) and stimulated with allogeneic mature dendritic (mDC) cells (5×10³ cells/well) and soluble anti-CD3 mAbs in the absence or presence of CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD4⁺ΔNGFR⁻ T cells or CD8⁺ΔNGFR⁻ T cells. FIGS. 7A and 7B show effects of CD4^{IL-10} cells from six different, unpooled, donors (Donor-C, Donor-E, and Donor-F in FIG. 7A and Donor-H, Donor-I, and Donor-L in FIG. 7B) on CD4⁺ T cells with percentages of proliferation and suppression. FIGS. 8A and 8B show effects of CD4^{IL-10} cells from six different single donors (Donor-C, Donor-E, and Donor-F in FIG. 8A and Donor-H, Donor-I, and Donor-L in FIG. 8B) on CD8⁺ T cell proliferation.

The results demonstrated that CD4^{IL-10} cells from 6 different single donors, unpooled and tested separately, downregulated the proliferative responses of both allogeneic CD4⁺ and CD8⁺ T cells. The suppressive effects on the CD4⁺ T cells varied between 51% and 96%, while the suppressive effects on the CD8⁺ T cells varied between 62% and 73%.

### Production and Characterization of Polydonor CD4^{IL-10} Cells

CD4^{IL-10} cells were generated as described above and FIG. 3 using CD4⁺ cells from multiple donors. CD4^{IL-10} cells from each donor were stimulated by the second (TF2) and third (TF3) restimulation. After the third stimulation, CD4^{IL-10} cells from the three donors were pooled at a 1:1:1 ratio and stimulated with with immobilized CD3 (10 μg/mL) and soluble CD28 mAb (1 μg/mL) for 48 hrs.

Polydonor CD4^{IL-10} Cells have a Cytokine Production Profile which is Comparable to that of CD4^{IL-10} Cells of Individual Donors and Tr Cells.

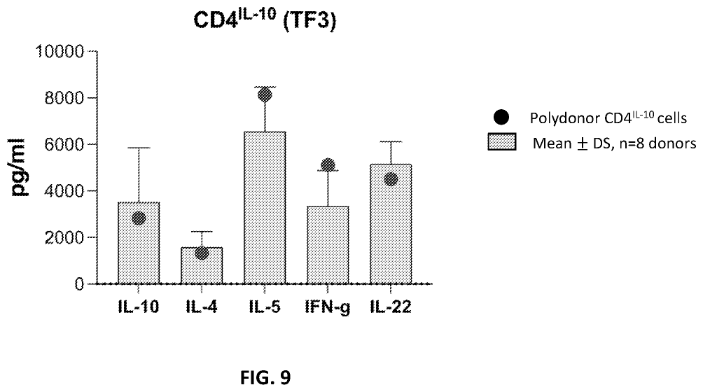

Culture supernatants were collected and levels of IL-10, IL-4, IL-5, IFN-γ and IL-22 were determined by ELISA. The results provided in FIG. 9 show that the cytokine production of polydonor CD4^{IL-10} cells pooled from 3 different allogeneic donors (pooled 1:1:1) (black dot) was comparable to that of CD4^{IL-10} cells from individual donor (n=8) derived CD4^{IL-10} cells (gray bars). The polydonor CD4^{IL-10} cells produced high levels of IL-10 and IL-22, variable levels of IL-5, IFN-γ and low levels of IL-4 and undetectable levels of IL-2 (not shown). These data indicate that it is feasible to pool CD4^{IL-10} cells and that these polydonor CD4^{IL-10} cells maintain the cytokine production signature of single donor derived CD4^{IL-10} cells and Tr1 cells. Importantly, the pooled allogeneic cell populations contained >95% viable cells indicating that they did not kill each other.

### Polydonor CD4^{IL-10} Cells Express High Levels of Granzyme B and Kill Myeloid Leukemia Cell Lines.

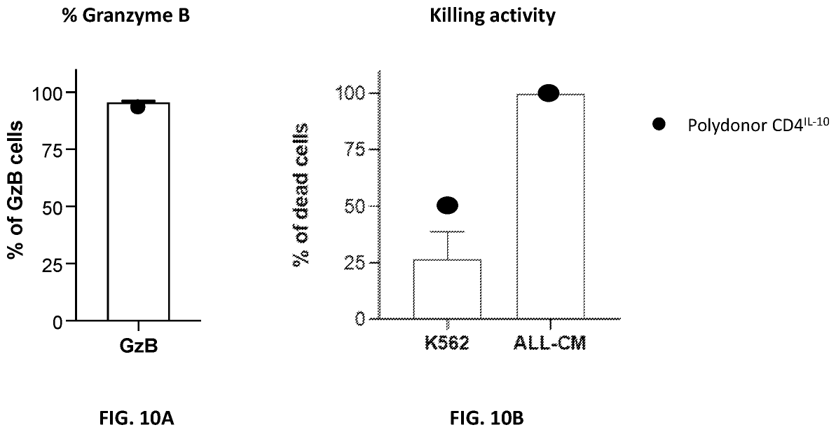

The polydonor CD4^{IL-10} cells were further analyzed after 3^{rd} round (TF3) of restimulation for expression of granzyme B (GzB). The data in FIG. 10A show that most of the polydonor CD4^{IL-10} cells express GzB. Over 95% of the polydonor CD4^{IL-10} cells expressed Granzyme B, comparable to the GzB expression of single donor derived CD4^{IL-10} cells (FIG. 10A).

The CD4^{IL-10} cells from 3^{rd} round (TF3) of restimulation were further analyzed for their cytotoxic effects on myeloid leukemia cells (ALL-CM cell line) or K562. The polydonor CD4^{IL-10} cells (10⁵/well) were co-cultured with K562 and ALL-CM cells (10⁵/well) at 1:1 ratio for 3 days. Residual leukemic cell lines (CD45^{low}, CD3⁻) were counted by FACS for each target cell. The results provided in FIG. 10B show that some level of cytotoxicity against K562 cells, which are highly sensitive for nonspecific cytotoxicity. Nevertheless, a level of selective killing of the polydonor CD4^{IL-10} cells (black dot) towards myeloid leukemia cells (ALL-CM) was obtained which is comparable to that of single donor derived CD4^{IL-10} cells (open bar).

Polydonor CD4^{IL-10} cells suppress the proliferative responses of both allogeneic CD4+ and CD8+ T cells.

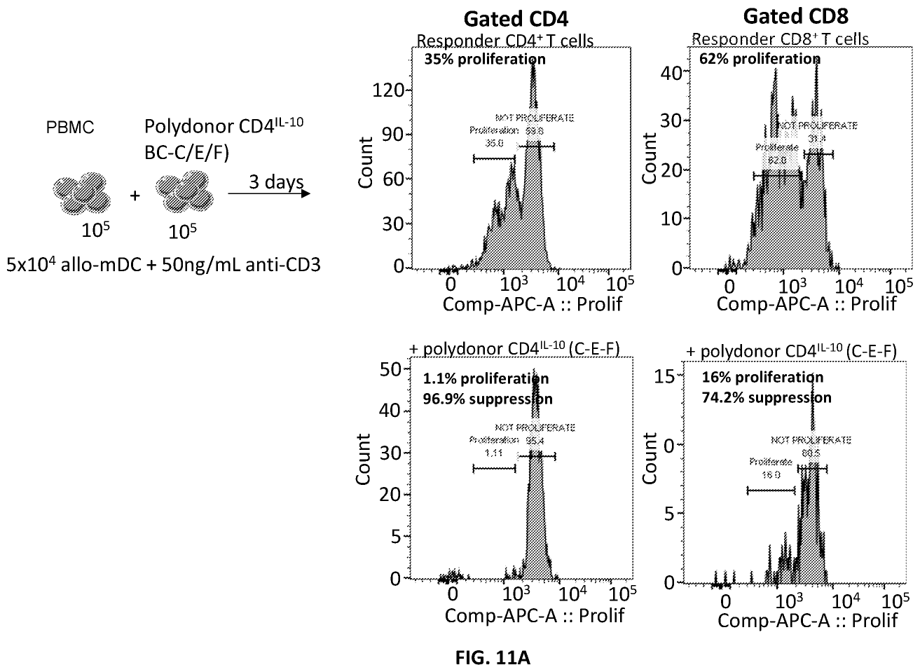

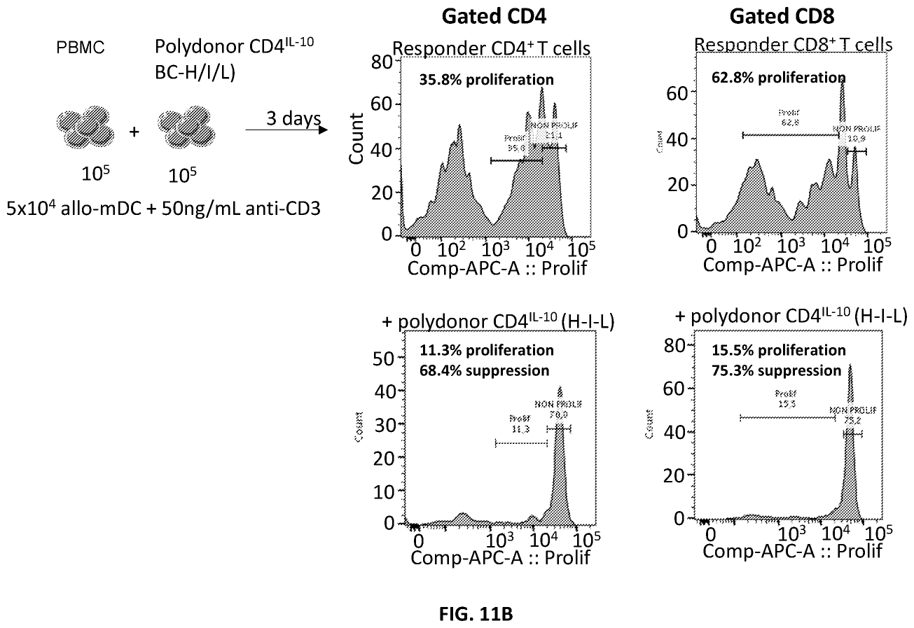

The polydonor CD4^{IL-10} cells were also analyzed for their effects on allogeneic CD4⁺ T cells or CD8⁺ T cells. Specifically, allogeneic PBMC cells were labeled with eFluor® 670 (5×10⁴ cells/well) and stimulated with allogenic mature dendritic (DC) cells (5×10³ cells/well) and soluble anti-CD3 mAbs in the absence or presence of polydonor CD4^{IL-10} cells (5×10⁴ cells/well) at a 1:1 Responder:Suppressor ratio. After 3 days of culture, the percentages of proliferating responder cells were determined by eFluor® 670 dilution with flow cytometry after gating on CD4⁺ΔNGFR⁻ T cells or CD8⁺ΔNGFR⁻ T cells. FIG. 11A shows results from polydonor CD4^{IL-10} cells containing CD4^{IL-10} cells from Donor-C, Donor-E, and Donor-F (C-E-F). FIG. 11B shows results from polydonor CD4^{IL-10} cells containing CD4^{IL-10} cells from Donor-H, Donor-I, and Donor-L (H-I-L), which had been frozen, stored and thawed prior to testing.

FIG. 11A shows that the polydonor CD4^{IL-10} cells (from 3 different donors) suppress CD4⁺ and CD8⁺ T-cell responses by 97% and 74%, respectively. Comparable results were obtained with a second, different batch of polydonor CD4^{IL-10} cells which was tested after the cells had been frozen, stored and thawed prior to testing (FIG. 11B). Suppression of CD4⁺ and CD8⁺ T cell proliferation was 68% and 75%, respectively. These data indicate that polydonor CD4^{IL-10} cells can be frozen, stored, and thawed without loss of function.

Collectively the data obtained with polydonor CD4^{IL10} cells indicate that these cell preparations can be pooled without any problems. They contain >95% viable cells and maintain all the relevant functions (cytokine production, cytotoxic capacity, and suppression of allogeneic T cell responses) of single donor CD4^{IL-10} cells. The use of larger pools of polydonor CD4^{IL-10} cells should reduce the natural variations observed between CD4^{IL-10} cell lots originating from different individual donors, and should provide a large quantity of off-the-shelf CD4^{IL-10} cells for human therapy.

A polydonor CD4^{IL-10} cell product will have significant advantages in terms of a more homogeneous product which will allow the determination of well defined, less lot-to-lot variation, potency, and release criteria. In addition, it will enable the development of a continuous large-scale cell production process.

### Other Methods for Production of Polydonor CD4^{IL-10} Cells

Before the lentiviral transduction, buffy coats from minimally 3-5 different donors are pooled. CD4⁺ cells are isolated from buffy coats by positive selection using anti-CD4 antibody. Purity of the pooled CD4⁺ cells is checked by FACS. Alternatively, frozen human CD4⁺ cells are obtained from minimally 3-5 normal healthy donors. The frozen human CD4⁺ cells are thawed before use. CD4⁺ cells from buffy coats or frozen stocks are activated for 24-48 hours by a combination of CD3 and CD28 antibodies or CD3− and CD28 antibody coated beads in the presence of IL-2. In some cases, CD4⁺ cells from buffy coats or frozen stocks are activated with soluble anti-CD3, soluble anti-CD28 mAbs, and rhIL-2 (50 U/mL) for 48 hours and transduced with a bidirectional lentiviral vector encoding for human IL-10 as described above for production of CD4^{IL-10} cells.

In some cases, the HLA haplotype of the T cell donors (or CD4⁺ cells isolated from the donors) are first determined and CD4⁺ cells having desired HLA haplotypes are selectively pooled and used.

Polydonor CD4^{IL-10} cells are generated by transducing the activated CD4⁺ cells described above with the lentiviral vector containing human IL-10 and ΔNGFR coding sequences described above.

On Day 7-11, which is 5-9 days after the transduction, the cells are harvested and successfully transduced T cells purified utilizing an anti-NGFR antibody. This process generally results in 95% pure populations of polydonor CD4^{IL-10} cells.

The purified polydonor CD4^{IL-10} cells are counted and re-stimulated by a mixture of CD3− and CD28 antibodies, CD3− and CD28 antibody coated beads, optionally in the presence of feeder cells for another 8-10 days in the presence of IL-2. In some cases, the purified polydonor CD4^{IL-10} cells are re-stimulated in the presence of feeder cells.

After a total culture period of 5 weeks, CD4^{IL-10} cells are harvested, counted and tested for their capacity to produce IL-10 spontaneously or following activation with CD3 and CD28 antibodies or CD3 and CD28 antibody coated beads. Additionally, the levels of GrzB and perforin are measured. Their capacity to suppress human T cell (PBMC) and purified CD4⁺ and CD8⁺ T cell proliferation are also tested.

In addition, the production of IL-22 is measured both constitutively and following activation of 200,000 CD4^{IL-10} cells in a volume of 200 microliter using a combination of CD3 and CD28 antibodies as described previously for the production of other cytokines such as IFNγ, IL-10, IL-4 and IL-5. IL-22 production levels are measured in IL-22 specific ELISA as described for the other cytokines in WO2016/146542. The pooled CD4^{IL-10} cells are frozen before storage.

### 6.7.3. Example 2: Treatment or Prevention of GvHD Using Polydonor CD4^{IL-10} Cells

### Effects of Polydonor CD4^{IL-10} Cells In Vivo.

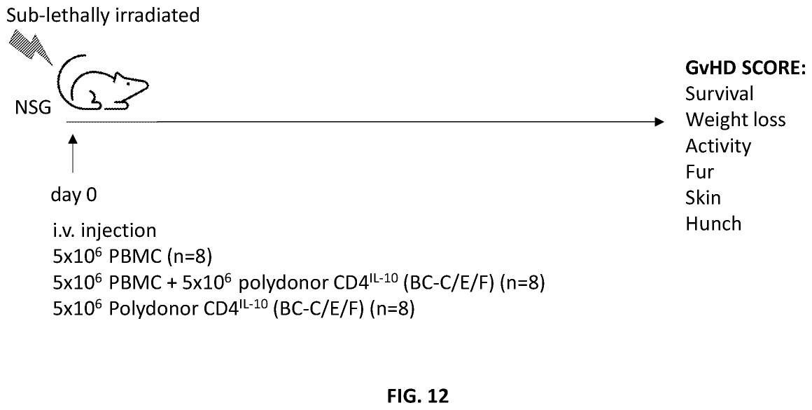

A population of polydonor CD4^{IL-10} cells were tested in a humanized xeno GvHD disease model, an NSG mouse model, for their effect on xeno-GvHD induced by human PBMC as illustrated in FIG. 12. NSG mice were sub-lethally irradiated and intravenously injected with (i) human PBMC (5×10⁶ cells/mouse), (ii) polydonor (three donors; BC-C/E/F) CD4^{IL-10} cells (5×10⁶ cells/mouse), or (iii) with human PBMC (5×10⁶ cells/mouse) in combination with polydonor CD4^{IL-10} cells (BC-C/E/F) (5×10⁶ cells/mouse). Xeno-GvHD was evaluated as previously described (Bondanza et al. Blood 2006) based on survival, weight loss (>20% weight loss), skin lesions, fur condition, activity, and hunch.

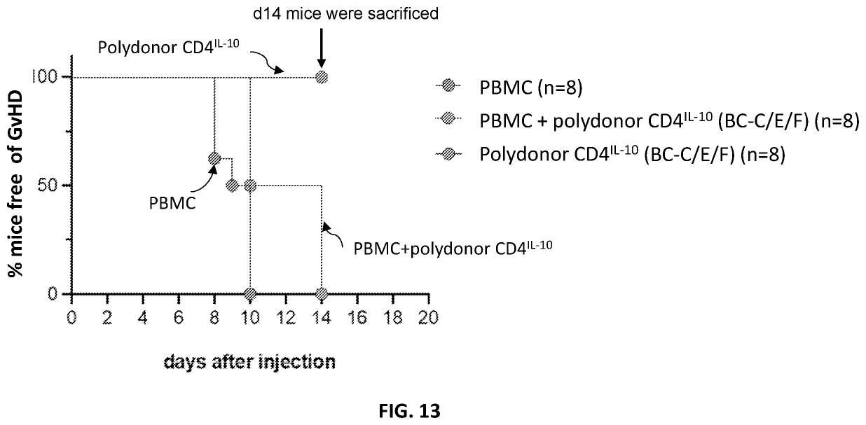

FIG. 13 shows % of NSG mice free of xeno-GvHD on each day after injection. Administration of 5×10⁶ human PBMC to irradiated NSG mice resulted unexpectedly in an unusually fulminant xeno-GvHD. All mice died at day 10 which reflects very lethal xeno-GvHD. Co-administration of 5×10⁶ polydonor CD4^{IL-10} cells delayed this fulminant xeno-GvHD, but the mice were sacrificed at day 14 because they reached the prespecified humane 20% body weight loss criterion for sacrifice (FIG. 13). Nevertheless, these results indicate that polydonor CD4^{IL-10} can delay extremely severe xeno-GvHD. Importantly, polydonor CD4^{IL-10} cells administered alone at the same dose as the PBMC (5×10⁶ cells) failed to induce any sign of xeno-GvHD.

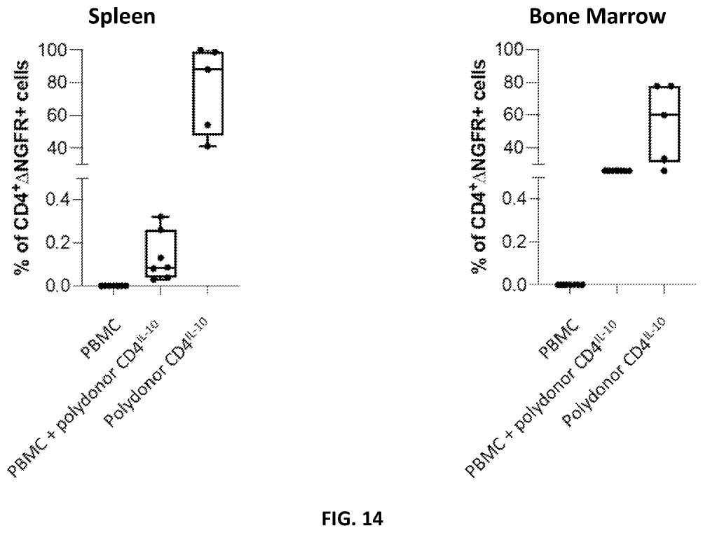

The presence of human CD4^{IL-10} cells was also tested in the spleen (FIG. 14, left panels) and bone marrow (FIG. 14, right panels) of the NSG mice injected with human PBMC (5×10⁶ cells/mouse), polydonor (three donors; BC-C/E/F) CD4^{IL-10} cells (5×10⁶ cells/mouse), or human PBMC (5×10⁶ cells/mouse) in combination with polydonor CD4^{IL-10} cells (three donors; BC-C/E/F)) (5×10⁶ cells/mouse) at 14 days post injection. The results provided in FIG. 14 show that polydonor CD4^{IL-10} cells migrated to spleen and bone marrow. Low percentages of these cells were found to be present 14 days after infusion of the cells. These results indicate that polydonor CD4^{IL-10} cells delayed fulminant xeno-GvHD induced by human PBMC and that they do not themselves induce any xeno-GvHD.

Polydonor CD4^{IL-10} Cells Inhibit Severe Xeno-GvHD by Purified CD4⁺ Cells.

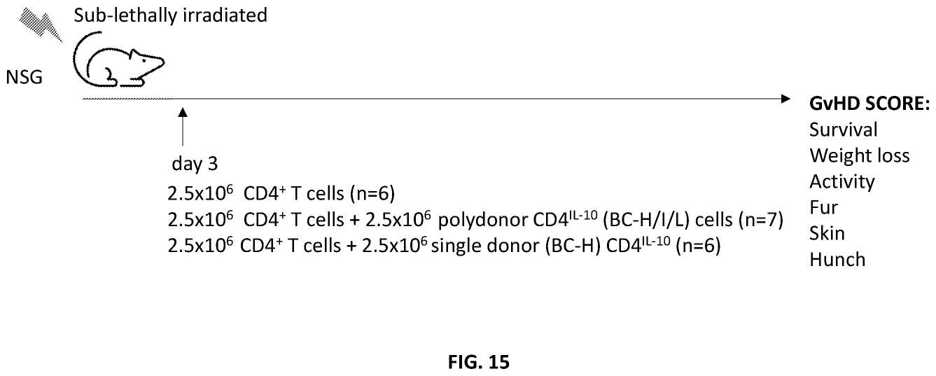

Polydonor CD4^{IL-10} cells were tested in a humanized xeno-GvHD model in which GvHD disease was induced by administration of 2.5×10⁶ purified human CD4⁺ T cells as illustrated in FIG. 15. NSG mice were sub-lethally irradiated at day 0 and on day 3 were intravenously injected with human CD4⁺ T cells (2.5×10⁶ cells/mouse) alone or in combination with polydonor CD4^{IL-10} cells (three different donors; BC-H/I/L) (2.5×10⁶ cells/mouse) or with CD4^{IL-10} cells from a single donor (BC-H) from the pool (2.5×10⁶ cells/mouse). Xeno-GvHD was evaluated as previously described (Bondanza et al. Blood 2006) based on survival, weight loss (>20% weight loss), skin lesions, fur condition, activity, and hunch.

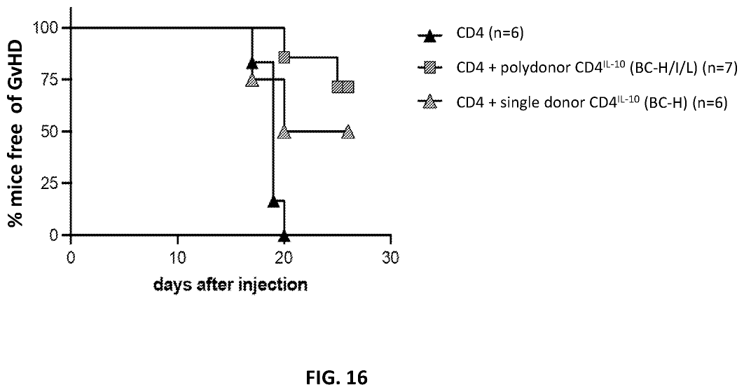

FIG. 16 shows % of NSG mice free of GvHD on each day after injection. The results show that polydonor CD4^{IL-10} (BC-H/I/L) cells can inhibit the xeno-GvHD mediated by human allogeneic CD4⁺ T cells. In this experiment, xeno-GvHD was very severe, because all mice in the control group which received CD4⁺ T cells were dead at day 20. In contrast, co-administration of 2.5×10⁶ polydonor CD4^{IL-10} inhibited GvHD by 75%. Single-donor CD4^{IL-10} cells were also protective but the effects were less potent.

### Other Experiments

Therapeutic effects of the polydonor CD4^{IL-10} cells are tested in four different groups of mice: (i) mice receiving human PBMC from a donor unrelated to the CD4^{IL-10} cells (xeno-GvHD positive control); (ii) mice receiving the polydonor CD4^{IL-10} cells (negative control); (iii) mice receiving a combination of PBMC and the polydonor CD4^{IL-10} cells at 1:1 ratio; and (iv) mice receiving a combination of PBMC and the polydonor CD4^{IL-10} cells at 2:1 ratio or at different ratios. Among animals receiving combination of PBMC and the polydonor CD4^{IL-10} cells, some animals receive PBMC and the polydonor CD4^{IL-10} cells concurrently, some animals receive polydonor CD4^{IL-10} cells several days (e.g., 5 days) after receiving PBMC, and some animals receive polydonor CD4^{IL-10} cells several days (e.g., 5 days) before receiving PBMC.

The mice are monitored for development of GvHD by measuring weight at weeks 1, 2, 3, 4, and if necessary week 5, after administration of PBMC and/or the polydonor CD4^{IL-10} cells. In addition to weight loss, the mice are inspected for skin lesions, fur condition and activity. The mice in the treatment groups are monitored for additional periods to determine effects of the polydonor CD4^{IL-10} cells on long term survival.

The amount and localization of the polydonor CD4^{IL-10} cells are also monitored in peripheral blood and tissues after administration. Specifically, presence of polydonor CD4^{IL-10} cells are monitored in peripheral blood and at sites of inflammation: spleen and bone marrow. Other sites presence of polydonor CD4^{IL-10} cells are monitored include lymph nodes and gut. The mice in the treatment group(s) are monitored for an additional 3 weeks to determine long-term survival.

The results demonstrate that polydonor CD4^{IL-10} cells are effective in reducing and preventing xeno-GvHD.

### 6.7.4. Example 3: Inhibition of GvHD and Treatment of Cancer

A population of polydonor CD4^{IL-10} cells are tested in an NSG mouse model transplanted with human PBMC and AML tumor cells for their effect on xeno-GvHD induced by human PBMC and anti-tumor effects. AML cells (ALL-CM) are administered i.v. as described previously in WO 2016/146542. PBMC or polydonor CD4^{IL-10} cells or combinations thereof are administered 3 days later.

Polydonor CD4^{IL-10} cells are obtained as described in Example 1. Therapeutic effects of the polydonor CD4^{IL-10} cells are tested in four different groups of mice, each having received irradiation and 5×10⁶ ALL-CM cells (AML mice) at day 0: (i) AML mice without additional treatment; (ii) AML mice receiving 5×10⁶ human PBMC from a donor unrelated to the polydonor CD4^{IL-10} cells—the PBMCs cause severe xeno-GvHD; (iii) AML mice receiving 2.5×10⁶ polydonor CD4^{IL-10} cells; and (iv) AML mice receiving combinations of PBMC and the polydonor CD4^{IL-10} cells at 1:1 or 2:1 ratio or at different ratios. One additional group of mice do not receive ALL-CML cells but receive 5×10⁶ human PBMC at day 3 after irradiation.

Effects of the polydonor CD4^{IL-10} cells on xeno-GvHD induced by human PBMC are tested based on weight loss, skin lesions, fur condition, activity, death rate and long-term survival. Anti-tumor or graft versus leukemia (GvL) effects of the polydonor CD4^{IL-10} cells are tested based on reduction of tumor cells in the circulation and long-term tumor free survival.

Some mice are monitored for up to 7 weeks in order to monitor long-term survival and complete tumor remissions.

Results demonstrate that polydonor CD4^{IL-10} cells are effective in both inhibition of xeno-GvHD and treatment of cancer.

### 6.7.5. Example 4: Treatment of Cancer Using Polydonor CD4^{IL-10} Cells

A population of polydonor CD4^{IL-10} cells were tested in an ALL-CM leukemia model of T cell therapy in NSG mice.

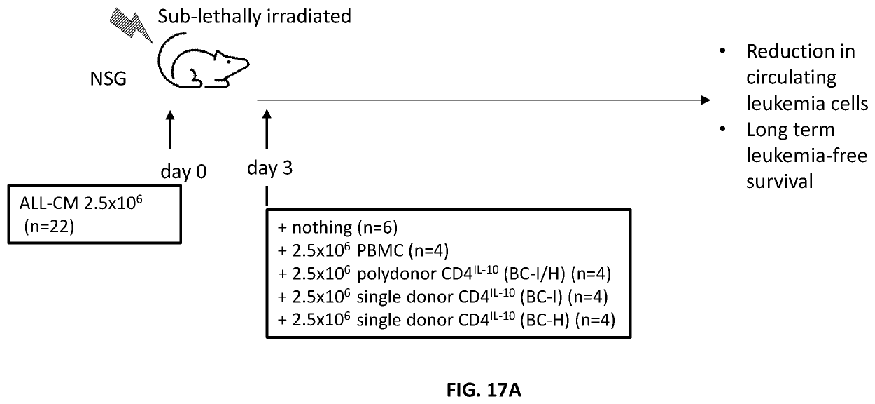

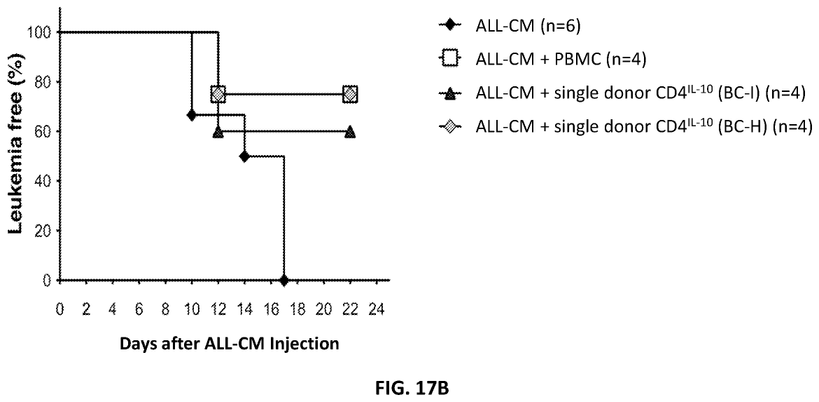

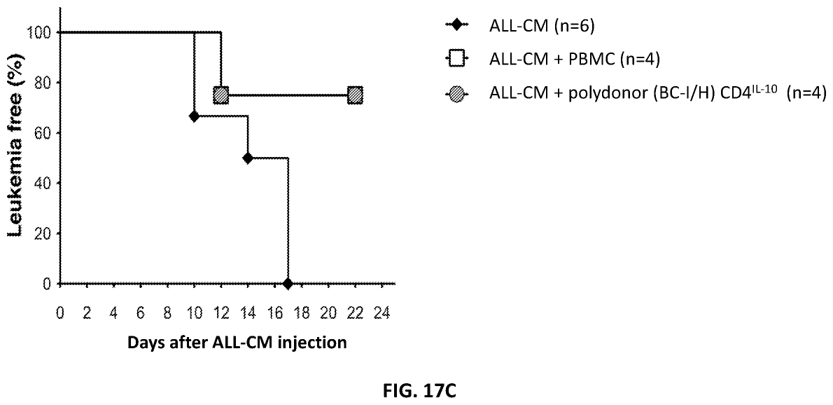

NSG mice were sub-lethally irradiated and intravenously injected with myeloid leukemia cells (ALL-CM) (2.5×10⁶) at day 0. In the first group of animals, no additional cells were administered. In the second group of animals PBMC (2.5×10⁶) were injected at day 3. In the third group of animals, polydonor CD4^{IL-10} cells (2.5×10⁶) were injected at day 3. In the fourth group of animals, single donor (from donor BC-I) CD4^{IL-10} cells (2.5×10⁶) were injected at day 3. In the fifth group of animals, single donor (from donor BC-H) CD4^{IL-10} cells (2.5×10⁶) were injected at day 3. Graft-versus-leukemia (GvL) effect was tested in the animals based on reduction of circulating leukemia cells and long-term leukemia free survival. Leukemia was measured as previously described (Locafaro G. et al Molecular Therapy 2017). See FIG. 17A.

As provided in FIG. 17B and FIG. 17C, all of the mice injected with ALL-CM myeloid leukemia cells alone had extensive leukemia progression at day 17. Administration of 2.5×10⁶ PBMC resulted in a strong inhibition of leukemia progression. Interestingly, a comparable level of inhibition of leukemia progression was obtained by both single-donor CD4^{IL10} (FIG. 17B) or polydonor CD4^{IL10} (FIG. 17C) cells. These data indicate that single donor and polydonor CD4^{IL10} have strong direct anti leukemia effects.

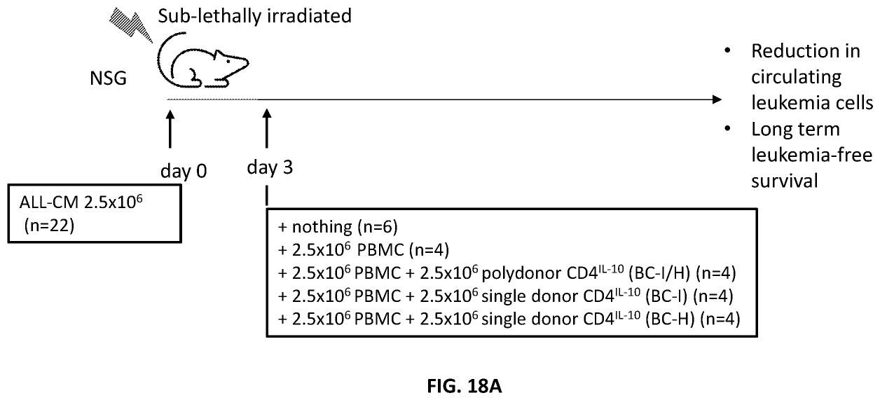

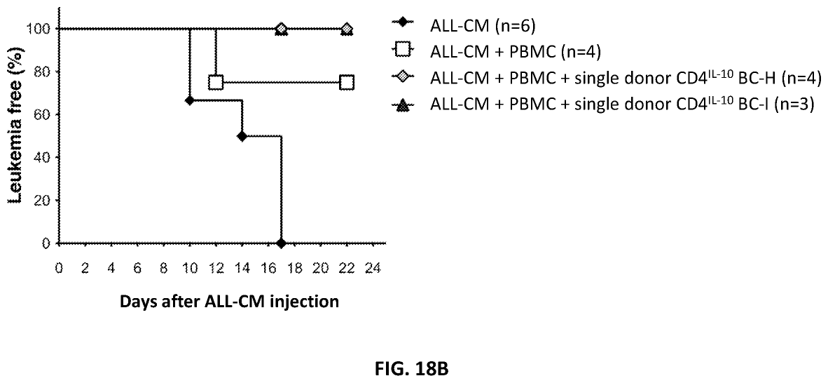

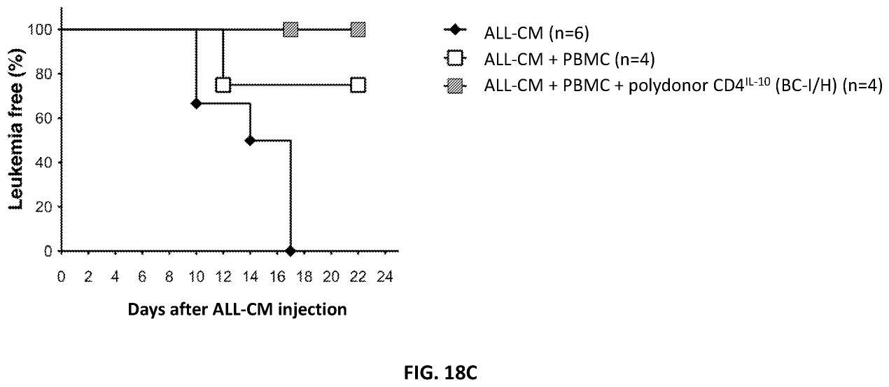

Graft-versus-leukemia (GvL) effects of single-donor CD4^{IL10} and polydonor CD4^{IL10} were further tested in combination with PBMC in mice injected with ALL-CM myeloid leukemia cells (FIG. 18A). Administration of 2.5×10⁶ PBMC resulted in a strong inhibition of leukemia progression. Administration of 2.5×10⁶ PBMC combined with single donor CD4^{IL10} (2.5×10⁶) cells resulted in a stronger inhibition of leukemia progression (FIG. 18B). Administration of 2.5×10⁶ PBMC combined with 2.5×10⁶ polydonor CD4^{IL10} cells had a comparable synergistic inhibition of leukemia effect to the single donor CD4^{IL10} (2.5×10⁶) cells (FIG. 18C). These data indicate that polydonor CD4^{IL10} cells do not interfere with the protective GVL effects of the PBMC, but act in synergy with the PBMC to mediate strong GvL effects.

### 6.7.6. Example 5: Treatment of Chronic Inflammatory and Autoimmune Diseases Using Polydonor CD4^{IL-10} Cells

Activation of the NLPR3 inflammasome has been implicated in many chronic inflammatory and autoimmune diseases. The NLPR3 inflammasome can be activated by “danger signals” which lead to caspase1-mediated production of the pro-inflammatory cytokines IL-1β and IL-18 by monocytes/macrophages. A series of in vitro experiments are performed to investigate the effects of polydonor CD4^{IL-10} cells on the NLPR3 inflammasome and IL-1 β/IL-18 production by human monocytes.

First, human PBMC are isolated from peripheral blood by standard density centrifugation on Ficoll/Paque (Sigma-Aldrich). Monocytes are isolated from the human PBMC by negative selection using monocyte isolation kit II (Miltenyi) according to the manufacturer's instructions. Negative selection is preferred because positive selection or adherence can lead to undesired activation of the cells. Isolated monocytes are plated at 5×10⁴ cells/200 μl in the presence of 2×10⁵ or 1×10⁵ polydonor CD4^{IL-10} cells/200 μl per well in 96-well microtiter plates in culture medium containing 3% toxin free human AB serum.

Table 1 summarizes treatment conditions applied to 9 sets of monocytes, each set including 6 wells of cells.

TABLE 1

| | | | | | |
|---|---|---|---|---|---|
| | | CD4 | | | |
| Group # | Monocytes | cells/supernatant | Inhibitors* | LPS** | Other*** |

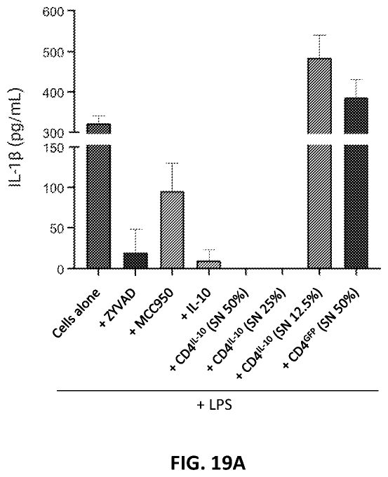

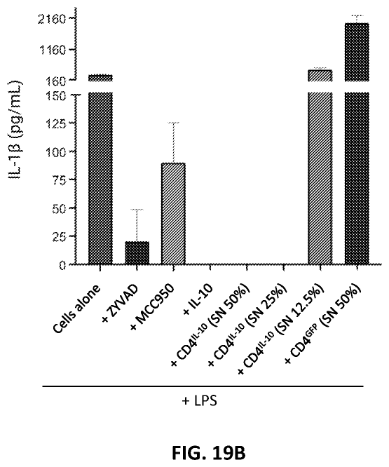

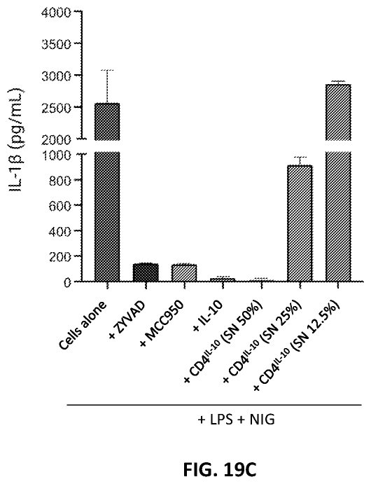

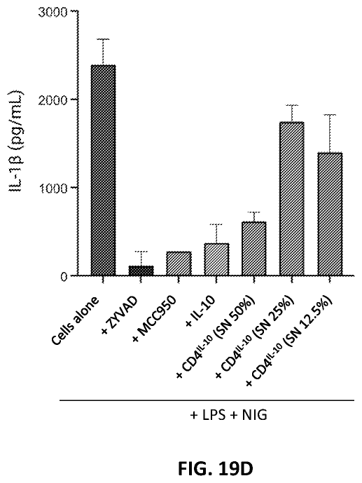

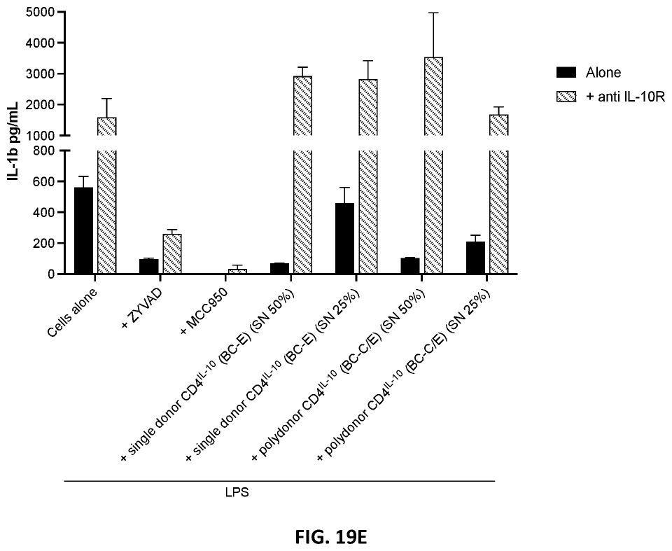

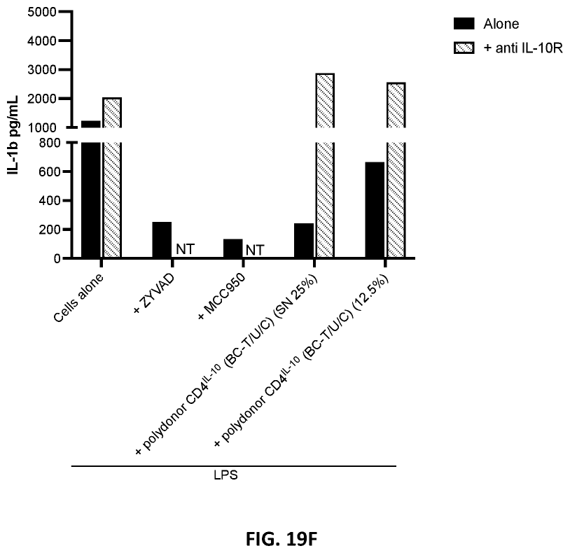

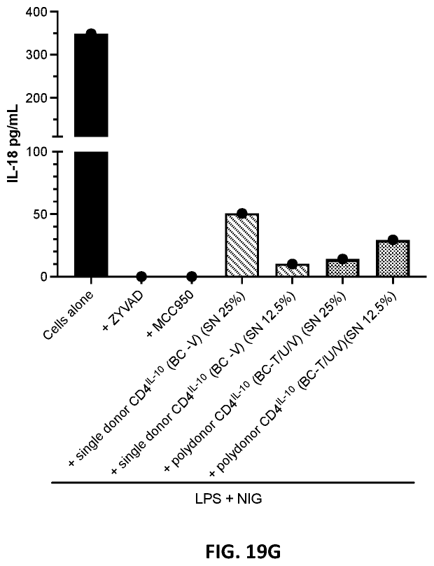

Medium control
Incubation time: 1 hour; followed by activation by
LPS +/− Nigericin as indicated in FIG. 19A-G

| | | | | |
|---|---|---|---|---|
| 1 | Monocytes | No | No | No LPS |
| 2 | Monocytes | No | No | LPS |
| 3 | Monocytes | No | Z-YVADfmk | LPS |
| 4 | Monocytes | No | MCC950 | LPS |

Culture of monocytes in the presence of supernatants**** obtained from
single donor or polydonor CD4^{IL-10} cell- or from CD4^{GFP} cell-culture

| | | | | | |
|---|---|---|---|---|---|
| 5 | Monocytes | 50% supernatant | No | LPS | |
| | | of CD4^{IL-10} cells | | | |
| 6 | Monocytes | 25% supernatant | No | LPS | |
| | | of CD4^{IL-10} cells | | | |
| 7 | Monocytes | 12.5% supernatant | No | LPS | |
| | | of CD4^{IL-10} cells | | | |
| 8 | Monocytes | 50% supernatant | No | LPS | Anti-IL-10 |
| | | of CD4^{IL-10} cells | | | receptor |
| | | | | | antibody |
| 9 | Monocytes | 50% supernatant | No | LPS | |
| | | of CD4^{GFP} cells | | | |

*Z-YVADfmk is an inhibitor specific to caspase 1. 20 uM Z-YVADfmk (Biovision, Enzo Life Sciences, or Axxora Life Sciences) dissolved in DMSO is used as indicated. MCC950 is an NLRP3 inhibitor. 10 uM MCC950 (Invivogen) is used as indicated.
**LPS (Sigma-Aldrich) 100 ng/mL plus nigericin as indicated (Nig, Invivogen) 10 uM added during the last 30 m of LPS incubation.
***Anti-IL-10R antibody (Biolegend) 30 ug/mL.
****Supernatants of CD4^{IL-10} and CD4 GFP cultures are obtained by incubating CD4^{IL-10} or CD4 GFP cells at 1 × 10⁶/mL for 3 days and collecting the supernatants. IL-10 production levels are measured by IL-10 specific ELISA.

After treatments outlined in Table 1, supernatants are collected from 6 wells for each group and IL-1 β/IL-18 production is measured by ELISA specific for mature IL-1β or IL-18 (Biolegend). Cells collected from 6 wells for select groups are analyzed by Western Blot to determine levels of activated caspase 1.

Data from the experiments show that polydonor CD4^{IL-10} cells down-regulate IL-1 and IL-18 production by activated monocytes. They further show that polydonor CD4^{IL-10} cells down-regulate mature caspase-1 production in activated monocytes. Additionally, polydonor CD4^{IL-10} and IL-10 produced by the polydonor CD4^{IL-10} down-regulate inflammasome.

Similar experiments are performed with human macrophages or dendritic cells instead of monocytes. Results from the experiments demonstrate that polydonor CD4^{IL-10} cells further down-regulate IL-1β, IL-18, and mature caspase-1 production from activated macrophages and dendritic cells.

These data suggest that polydonor CD4^{IL-10} cells can be used to treat diseases or disorders involving hyperactivation of NLPR3 inflammasome. In particular, polydonor CD4^{IL-10} cells can be used to treat chronic inflammatory and autoimmune diseases. The NLPR3 inflammasome can be activated by exogenous or endogenous “danger signals”, such as Pathogen Associated Molecular Patterns (PAMPs), silica, asbestos, Danger Associated Molecular Patterns (DAMPs) like products from damaged mitochondria, necrotic and stressed cells, and uremic acid crystals.

### 6.7.7. Example 6: Supernatant of Polydonor CD4^{IL-10} Cells Inhibit NLPR3 Inflammasome Activation and IL-1β and IL-18 Production by Human Monocytes

CD14⁺ monocytes were isolated from PBMC using a pan monocyte isolation kit (Miltenyi Biotec, Bergisch Gladbach, Germany) and plated in 96 flat microtiter wells at 2×10⁵/200 μL per well and cultured in the presence of LPS. The cells were cultured further in the presence of Z-YVADfmk (20 microMol), MMC950 (10 microMol), IL-10 (10 ng/mL) or various concentrations of single- or pooled donor CD4^{IL-10} cell supernatants as summarized in Table 1.

The supernatants were obtained from single- or pooled donor CD4^{IL-10} cells activated for 72 hours with a combination of CD3 and CD28 antibodies as described previously. (Andolfi et al. 2012, Mol. Therapy Vol. 20, 1778-1790, Locafaro et al. Mol Ther 2017, 25, 2254) In some cases (FIGS. 19C and 19D), the monocytes were incubated with LPS in combination with the NLPR3 inflammasome activator nigericin (“NIG”) which was added during the last 30 minutes of the LPS activation.

The NLPR3 inflammasome was activated by LPS, resulting in the production of mature caspase 1 and the biologically active forms of IL-1β and IL-18. Monocytes plated in the absence of LPS activation did not produce detectable levels of IL-10 during the incubation period (not shown).

Addition of the supernatant of single donor derived CD4^{IL-10} cells (containing 1769 pg IL-10/mL) inhibited IL-1β production by LPS activated monocytes from donor #1 and #2 at concentrations of 50%, 25% and 12.5% respectively, in a dose dependent fashion (FIG. 19A and FIG. 19B). Supernatant of CD4+ cells transduced with GFP was used as a control. Complete inhibition of IL-1β production is observed at concentrations of 50% and 25%, whereas supernatants of GFP transduced control CD4+ cells at concentrations of 50% were ineffective.

Various concentrations of CD4^{IL-10} T cell supernatant (50%, 25% or 12.5%), Z-YVADfmk or MCC950 were further tested on monocytes activated with LPS and nigericin (“NIG”). Supernatants from single donor (BC-E) or pooled donor CD4^{IL-10} cells contained 5295 or 3532 pg IL-10/mL respectively. Supernatants of single donor CD4^{IL-10} cells were also very effective in inhibiting LPS induced IL-1β production enhanced by the NLPR3 inflammasome activator nigericin (FIG. 19C and FIG. 19D).

The data demonstrate that the supernatants from CD4^{IL-10} cells at concentrations of 50% were as effective as the irreversible caspase 1 inhibitor Z-YVADfmk (Guo et al. 2015, Nature Med 21, 677), the selective NLPR3 inflammasome inhibitor MCC950 (Coll et al. 2019, Nature Chem. Biol 15,556) and recombinant IL-10, indicating that IL-10 containing supernatants inhibit NLPR3 inflammasome activation and mature caspase1 production resulting in strong inhibition of the production of the proinflammatory cytokine IL-1β (FIG. 19A-19D).

Comparable results were obtained in second series of experiments with supernatants of single donor (BC-E) and pooled CD4^{IL-10} cells from 2 different donors (BC-C/E). The CD4^{IL-10} cells were activated by a combination of CD3 and CD28 antibodies as described (Andolfi et al. 2012). After 3 days the supernatants from the CD4^{IL-10} cells were collected. These supernatants contained 5295 and 3532 pg IL-10/mL respectively, and inhibited LPS induced IL-1β production by monocytes from donor #3 in a dose dependent fashion (FIG. 19E). Supernatants at concentrations of 50% were as effective as Z-YVADfmk and MCC950. The inhibitory effects of the supernatants of the pooled CD4^{IL-10} cells were completely neutralized by an anti-IL-10 receptor antibody. Similarly, supernatants pooled from 3 different donors containing 2589 pgIL-10/mL, dose dependently inhibited IL-10 production by monocytes from donor #4 (FIG. 19F). The inhibitory effects of the supernatants are completely neutralized by an IL-10 receptor antibody demonstrating that NLPR3 activation is mediated by IL-10. The results indicate that production of the pro inflammatory cytokine IL-10 is strongly inhibited by IL-10 produced by the polydonor CD4^{IL-10} T cells. As expected, the anti IL-10 receptor antibody had no effect of the inhibition of IL-1 production mediated by Z-VADfmk and MCC950 (FIG. 19E).

CD4^{IL-10} T cells were further tested on monocytes from donor #4 activated by LPS and nigericin. Various concentrations of single donor (BC-V) or polydonor (three donors; BC-T/U/V) CD4^{IL-10} cell supernatants containing 2583 or 2589 pg IL-10/mL respectively, ZYVADfmk or MCC950 were tested. Data provided in FIG. 19G show that pooled supernatants of 3 different donors (BC T-U-V) down regulate IL-18 production induced by LPS in combination with nigericin.

Collectively, these data indicate that IL-10 produced by single- and poly donor CD4^{IL-10} cells strongly down regulates the NLPR3 inflammasome resulting in strong inhibition of the pro inflammatory cytokines IL-1β and IL-18. 6.7.8. Example 7: Single-donor and polydonor CD4^{IL-10}° cells inhibit xeno GvHD and myeloid tumor growth in vivo

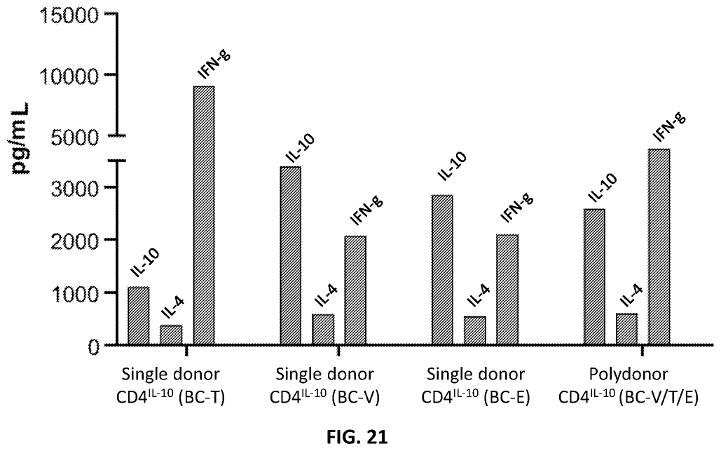

Functional properties and quality of the single donor (BC-T, BC-V, and BC-E) or polydonor (BC-V/T/E) CD4^{IL-10} cells were tested as described in Andolfi et al. Mol Ther 2012, 20, 177 and Locafaro et al. Mol Ther 2017, 25, 2254. Both single donor- and polydonor CD4^{IL-10} cells produced high levels of IL-10, variable levels of IFN-γ, very low levels of IL-4 and no detectable IL-2 (the latter not shown), reflecting the characteristic cytokine production profile of Tr1 cells (FIG. 21).

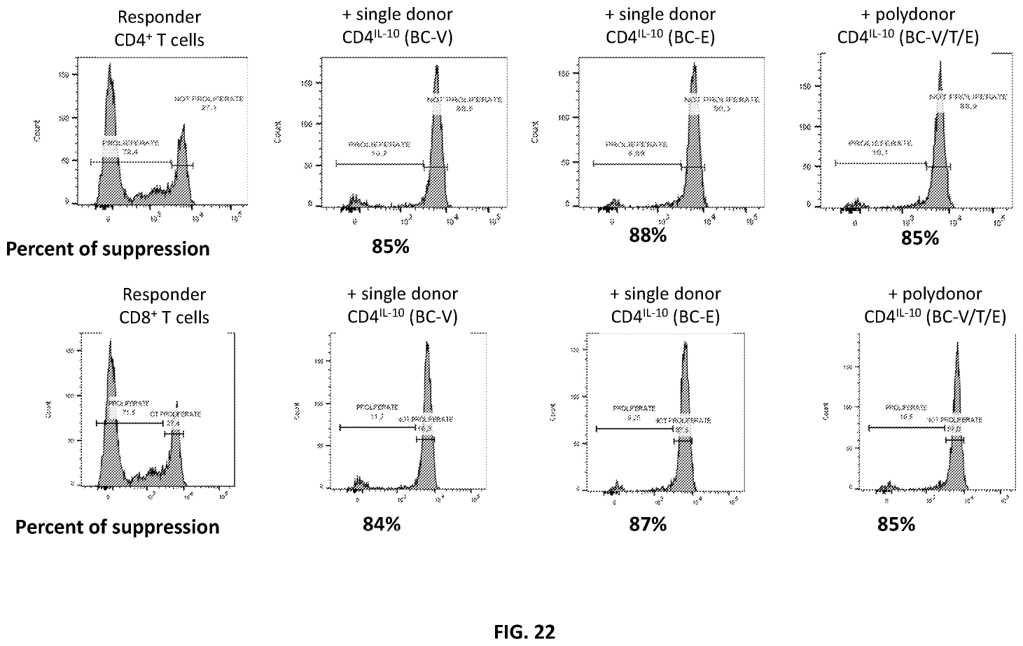

Further, the suppressive capacity of the single donor (BC-T, BC-V, and BC-E) or polydonor (BC-V/T/E) CD4^{IL-10} cells on CD4⁺ and CD8⁺ T cell proliferation was measured in vitro on allogeneic PBMC. PBMC were labeled with eFLuor670 (Invitrogen). Labeled PBMC (1×10⁵) were activated with immobilized CD3 (10 μg/mL) and soluble CD28 antibodies (1 μg/mL). Single and polydonor CD4^{IL-10} cells were added at a 1:1 ratio in a final volume of 0.2 mL in 96 well round bottom plates. After 4 days of co-culture, their suppressive effects on the proliferation of eFluor670 labeled responder cells was determined by eFluor670 dilution using flow cytometry as described (Locafaro et al. Mol Ther 2017, 25, 2254). FIG. 22 provides results from the flow cytometry. Single- and polydonor CD4^{IL-10} cells strongly inhibited in vitro proliferation of both allogeneic CD4+ and CD8+ T cells by more than 80% (FIG. 22).

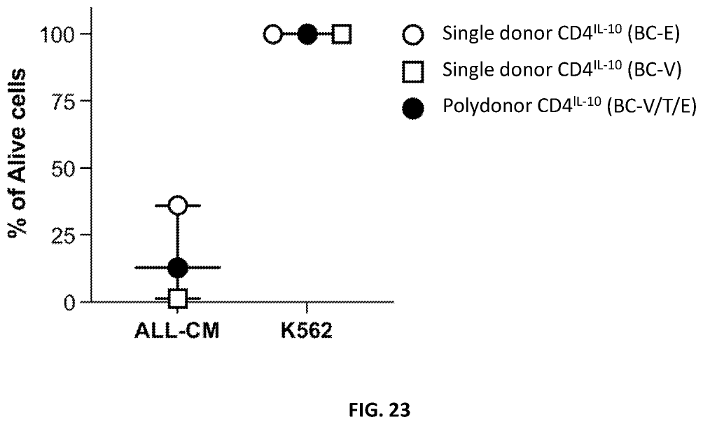

The CD4^{IL-10} cells were further analyzed for their cytotoxic effects against myeloid leukemia cells (ALL-CM) and an erythroid leukemia cell line (K562). Single (BC-E and BC-V) or polydonor (BC-V/T/E) CD4^{IL-10} cells were co-cultured at a 1:1 ratio with ALL-CM or K562 cells. After 3 days the cells were harvested and surviving CD45^{low} CD3⁻ target cells were counted and analyzed by FACS as described ((Locafaro et al. Mol Ther 2017, 25, 2254). The single donor and poly donor CD4^{IL-10} cells also mediated strong direct cytotoxic effects on ALL-CM myeloid tumor cells, whereas they failed to kill the sensitive K562 cells, which lack Class I MHC expression required for their cytotoxic activity (FIG. 23). Single (BC-E and BC-V) or polydonor (BC-V/T/E) CD4^{IL-10} cells had comparable cytotoxic activities against these two target cell lines (ALL-CM and K562).

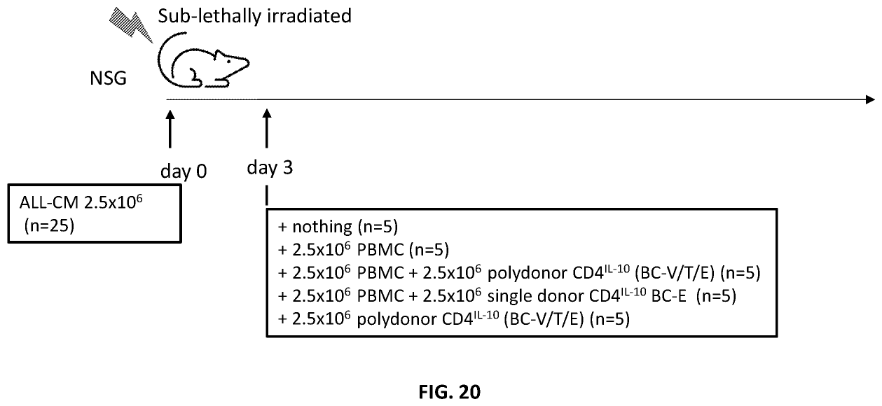

Cytotoxic effects of single-donor (BC-E) and polydonor (BC-V/T/E) CD4^{IL-10} cells were also tested in vivo, using a humanized xeno GvHD disease model—an NSG mouse intravenously injected with ALL-CM cells (2.5×10⁶). Their effect on GvHD induced by human PBMC from an allogeneic donor as well as their effect on the growth of acute myeloid leukemia in cell line ALL CM in a therapeutic setting were tested as illustrated in FIG. 20.

Eight to ten-week-old female NOD scid gamma, (NSG) mice were obtained from Charles-River Italia (Calco, Italy). The experimental protocol was approved by the internal committee for animal studies of the Ospedale San Raffaele (Institutional Animal Care and Use Committee (IACUC). At day 0, the mice received total body irradiation from a linear accelerator. ALL-CM cells (2.5×10⁶) were injected at day 0. On day 0, different groups of mice were injected with nothing, allogeneic PBMC (2.5×10⁶), single donor (BC-E, 2.5×10⁶) or polydonor CD4^{IL-10} cells pooled at 1:1:1 ratio from 3 different donors (BC-V/T/E, 2.5×10⁶) in combination with allogeneic PBMC (2.5×10⁶) or polydonor CD4^{IL-10} cells (2.5×10⁶) on day 3. All cells were administered i.v. in volumes of 250 μl of Iscove's modified Dulbecco's medium. Mice were monitored 3-4 times per week.

The NSG mice were divided into five cohorts of 5 mice and each group was treated on day 0 with (i) none as a control; (ii) allogeneic mononuclear cells (PBMC); (iii) allogeneic PBMC and polydonor CD4^{IL-10} cells (BC-V/T/E); (iv) allogeneic PBMC and single-donor CD4^{IL-10} cells (BC-E); or (v) polydonor CD4^{IL-10} (BC-V/T/E) cells administered at day 3 Myeloid leukemia progression was measured as previously described ((Locafaro et al. Mol Ther 2017, 25, 2254).

Administration of ALL-CM cells to NSG mice resulted in a rapid expansion of these cells and all the mice died or had to be sacrificed on day 20. Injection of PBMC prevented leukemia progression as expected. Both single- and polydonor CD4^{IL10} cells given in combination with allogeneic PBMC did not interfere with anti myeloid leukemia effects of the PBMC.

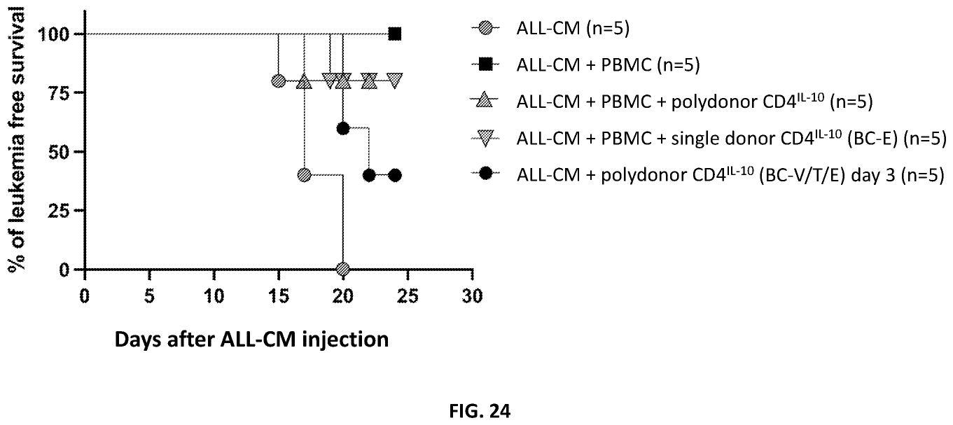

Injection of polydonor CD4^{IL-10} (BC-V/T/E) cells 3 days after administration of the ALL-CM cells (when already massive expansion of these cells is ongoing) resulted in inhibition of tumor growth. These results indicate that polydonor CD4^{IL-10} cells have direct therapeutic anti myeloid leukemia effects in vivo (FIG. 24).

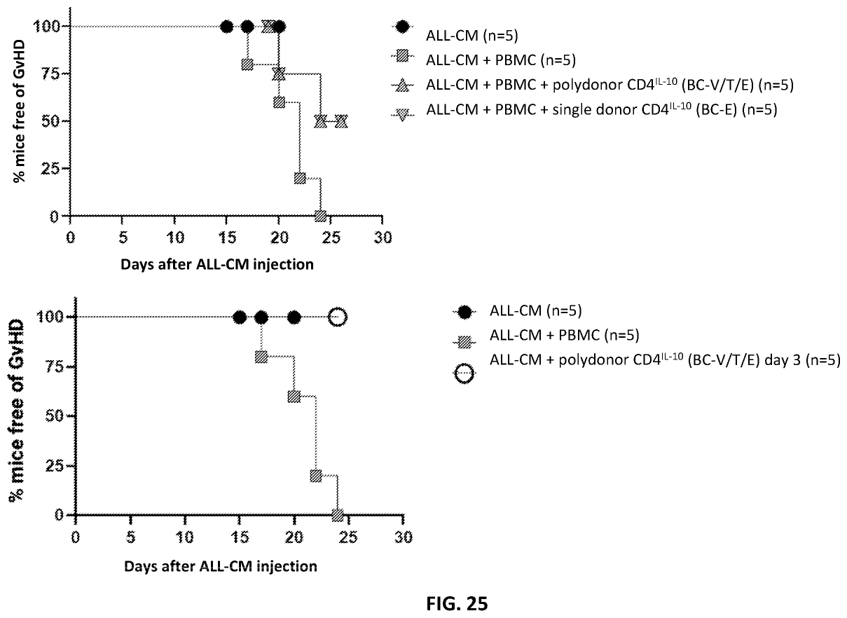

However, despite their beneficial anti myeloid leukemia effects, the PBMC induced a very severe form of xeno-GvHD and all mice died by day 24 (FIG. 25). In the study, single-donor (BC-E) and polydonor (BC-V/T/E) CD4^{IL-10} cells were tested on their capacity to inhibit xeno-GvHD induced by PBMC following administration in NSG mice. On day 0, the NSG mice were injected with ALL-CM cells (2.5×10⁶). The mice were divided into five groups and each group was treated with (i) none as a control; (ii) allogeneic mononuclear cells (PBMC); (iii) allogeneic PBMC and polydonor (BC-V/T/E) CD4^{IL-10} cells; (iv) allogeneic PBMC and single-donor CD4^{IL-10} cells (BC-E) or polydonor (BC-V/T/E) CD4^{IL-10} cells injected at day 3. In the animals, xeno-GvHD was measured by survival and weight loss. In addition, hunching, fur condition and skin integrity were monitored as described (Bondanza et al, Blood, 2006, 107, 1828). If weight loss reached more than 20%, the mice were sacrificed for ethical reasons. FIG. 25 shows % of NSG mice free of GvHD in each day following day 1 injection with ALL-CM cells (2.5×10⁶) and subsequent treatment with PBMC with or without single-donor or polydonor CD4^{IL-10} cells.

The results show that polydonor CD4^{IL-10} cells did not induce xeno-GvHD, and down regulated xeno-GvHD induced by allogeneic PBMC. Collectively these results indicate that polydonor CD4^{IL-10} cells downregulate severe xeno-GvHD, have direct anti myeloid leukemia effects in a therapeutic setting and do not interfere with the protective anti myeloid leukemia effects of the PBMC.

### 6.7.9. Example 8: Adoptive Transfer of Polydonor CD4^{IL-10} Cells Derived from Four Different Donors

Adoptive transfer of polydonor CD4^{IL-10} cells derived from four different donors was tested for the transfer's ability to inhibit PBMC-induced xeno-GvHD.

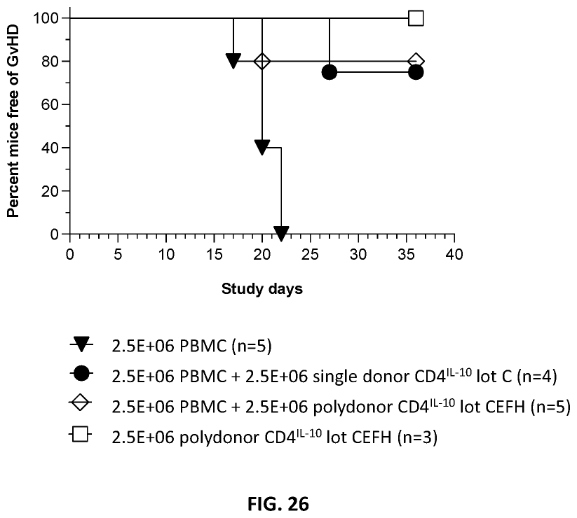

In these experiments, single-donor CD4^{IL-10} cells (donor C; lot C) and polydonor CD4^{IL-10} cells derived from 4 different donors (donors C, E, F, and H; lot CEFH) were tested in a humanized mouse model of GvHD induced by allogeneic PBMC. In this model, NSG mice were sub-lethally irradiated at day 0 and injected at day 3 (slow bolus i.v.) with (i) 2.5E+06 allogeneic PBMC, (ii) 2.5E+06 allogeneic PBMC in combination with 2.5E+06 single-donor CD^{4IL-10} cells (lot C), (iii) 2.5E+06 allogeneic PBMC in combination with 2.5E+06 cells polydonor CD4^{IL-10} cells (lot CEFH), or (iv) 2.5E+06 cells polydonor CD4^{IL-10} cells (lot CEFH) alone. Xeno-GvHD was determined using a composite score of weight loss, fur appearance, skin appearance, hunch, and activity (see Bondanza A, et al. Blood 2006; 107:1828-36]. As shown in FIG. 26, only mice administered with the polydonor CD4^{IL-10} cells were 100% free of xeno-GvHD at the end of the study.

In summary, this data demonstrated that adoptive transfer of polydonor CD4^{IL-10} cells derived from four different donors inhibits PBMC-induced xeno-GvHD and does not induce xeno GvHD.

### 6.7.10. Example 9: Generation of a Variant of IL-10

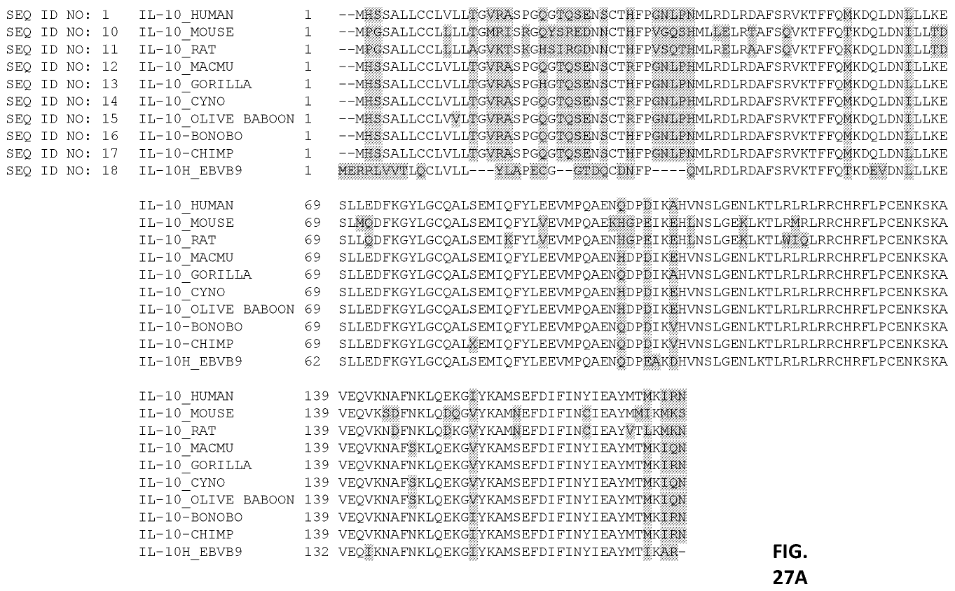

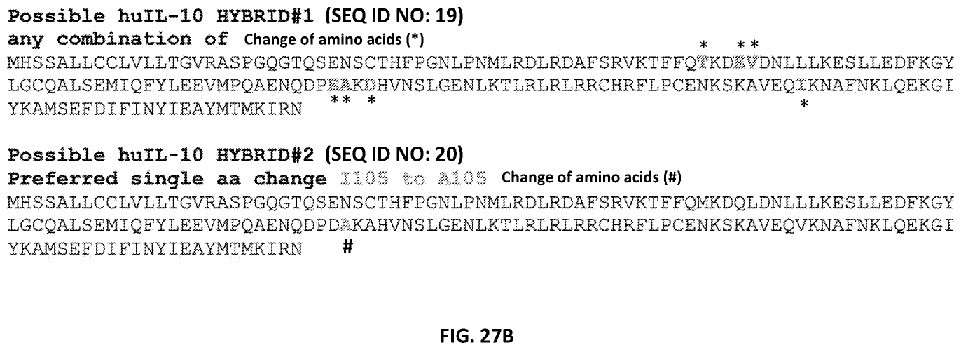

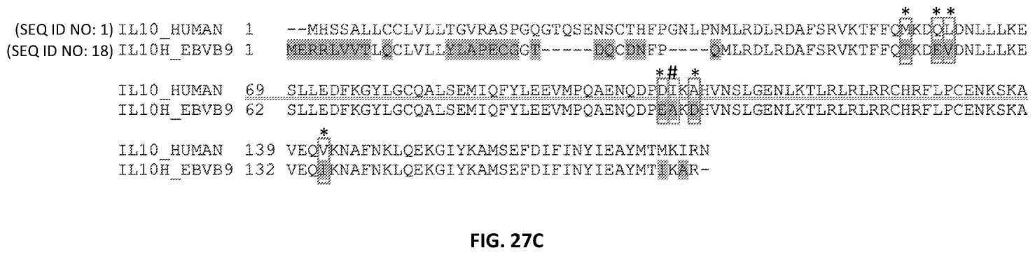

Variants of human IL-10 are generated by introducing amino acid modification(s) (e.g., substitution, insertion, deletion) in view of IL-10 sequences of other species. Modification sites are determined by sequence alignment as provided in FIG. 27A. Amino acid positions having different amino acids among species are identified from the alignment and modified by introducing substitution, insertion, or deletion of amino acids.

Two examples of the variant of human IL-10 are provided in FIG. 27B. Possible huIL-10 HYBRID #1 (SEQ ID NO: 19) is generated by substituting three amino acids (D, I and A) of human IL-10 with three different amino acids (E, A, and D) of viral IL-10 (EBVB9) at the corresponding positions. Possible huIL-10 HYBRID #2 (SEQ ID NO: 20) is generated by substituting one amino acid (I105) of human IL-10 with another amino acid (A105) of viral IL-10 (EBVB9) at the corresponding position. FIG. 27C shows alignment of human IL-10 (SEQ ID NO: 1) with IL10 EBVB9 (SEQ ID NO: 18) with “*” indicating the one or more amino acid positions that are substituted in IL-10 hybrid #1 and “#“indicating the preferred I105 to A105 amino acid substitution for IL-10 hybrid #2.

The variants of human IL-10 are cloned into an expression vector as described in the above section and tested for the expression and function of the variant proteins. Selected variants of human IL-10 are used to generate CD4^{IL-10} cells. Efficiency of CD4^{IL-10} cells are tested as provided herein.

### 6.7.11. Experimental Methods and Materials

Cell preparation and cell lines. Peripheral blood mononuclear cells (PBMC) were prepared by centrifugation over Ficoll-Hypaque gradients. CD4⁺ T cells were purified with a CD4 T cell isolation kit (Miltenyi Biotec, Bergisch Gladbach, Germany) with a resulting purity of >95%. Mature dendritic cells (DC) were generated from peripheral blood CD14⁺ monocytes positively selected using CD14⁺ MicroBeads (Miltenyi Biotech, Germany) according to the manufacturer's instructions and cultured in RPMI 1640 (Lonza, Italy) supplemented with 10% fetal bovine serum (FBS; Lonza, Italy), 100 U/mL penicillin/streptomycin (Lonza, Italy), 2 mM L-glutamine (Lonza, Italy), at 37° C. in the presence of 10 ng/mL recombinant human (rh) IL-4 (R&D Systems, Minneapolis MN, USA) and 100 ng/mL rhGM-CSF (Genzyme, Seattle, WA, USA) for 5 days and matured with 1 mg/mL of lipopolysaccharide (LPS, Sigma, CA, USA) for an additional two days.

Plasmid construction. The coding sequence of human IL-10 was excised from pH15C (ATCC n°⁰ 68192), and the 549 bp fragment was cloned into the multiple cloning site of pBluKSM (Invitrogen) to obtain pBluKSM-hIL-10. A fragment of 555 bp was obtained by excision of hIL-10 from pBluKSM-hIL-10 and ligation to 1074.1071.hPGK.GFP.WPRE.mhCMV.dNGFR.SV40PA (here named LV-ΔNGFR), to obtain pLVIL-10. The presence of the bidirectional promoter (human PGK promoter plus minimal core element of the CMV promoter in opposite direction) allows co-expression of the two transgenes (Locafaro et al. Mol Ther. 2017; 25(10):2254-2269). The sequence of pLVIL-10 was verified by pyrosequencing (Primm).

Vector production and titration. VSV-G-pseudotyped third generation bidirectional lentiviral vectors were produced by Ca₃PO₄ transient four-plasmid co-transfection into 293T cells and concentrated by ultracentrifugation as described (Locafaro et al. Mol Ther. 2017; 25(10):2254-2269). Titer was estimated by limiting dilution, vector particles were measured by HIV-1 Gag p24 antigen immune capture (NEN Life Science Products; Waltham, MA), and vector infectivity was calculated as the ratio between titer and particle. Titers ranged from 5×10⁸ to 6×10⁹ transducing units/mL, and infectivity from 5×10⁴ to 10⁵ transducing units/ng of p24.

Generation of CD4^{IL-10} cell lines. Polyclonal CD4-transduced cells were obtained as previously described (Andolfi et al. Mol Ther. 2012; 20(9):1778-1790, Locafaro et al. Mol Ther 2017, 25, 2254). Briefly, CD4 purified T cells were activated for 48 hours with soluble anti-CD3 monoclonal antibody (mAb, 30 ng/mL, OKT3, Janssen-Cilag, Raritan, NJ, USA), anti-CD28 mAb (1 pg/mL, BD) and rhIL-2 (50 U/mL, PROLEUKIN, Novartis, Italy). T cells were transduced with LV-IL-10/ΔNGFR (CD4^{IL-10}) with multiplicity of infection (MOI) of 20. At day 11, CD4⁺ΔNGFR+ cells were beads-sorted using CD271⁺ Microbeads (Miltenyi Biotec, Bergisch Gladbach, Germany) and expanded in X-VIVO15 medium with 5% human serum (BioWhittaker-Lonza, Washington), 100 U/mL penicillin-streptomycin (BioWhittaker), and 50 U/mL rhIL-2 (PROLEUKIN, Novartis, Italy). At day 7 and 10, medium was replaced by fresh medium supplemented with 50 U/mL of rhIL-2. At day 14, cells were collected, washed, and restimulated with allogeneic feeder mixture as previously described (Locafaro et al. Mol Ther 2017, 25, 2254). After 14 days, cells were collected and frozen. Thawed CD4^{IL-10} cells were restimulated and after the 2^{nd} and 3^{rd} re-stimulation and expansion were functionally characterized in vitro and used for in vivo experiments.

Vector Copy Number Analysis. Cells were cultured for at 11 days after transduction in order to get rid of non-integrated vector forms. Genomic DNA was isolated with QIAamp DNA Blood Mini Kit (QIAGEN, 51106), according to the manufacturer's instructions. Vector integrations were quantified by QX200 Droplet Digital PCR System (Bio-Rad), according to the manufacturer's instructions.

Cytokine determination. To measure cytokine production, after 2^{nd} and 3^{rd} re-stimulation single donor and polydonor CD4^{IL-10} cells were left unstimulated or stimulated with immobilized anti-CD3 (10 pg/mL) and soluble anti-CD28 (1 pg/mL) mAbs in a final volume of 200 μL of medium (96 well round-bottom plates, 2×10⁵/well). Supernatants were harvested after 48 hours of culture and levels of IL-10, IL-4, IL-5, IFN-γ and IL-22 were determined by ELISA according to the manufacturer's instructions (BD Biosciences).

Flow cytometry analysis. For the expression of Granzyme B (clone MHGB04, Invitrogen, USA) after surface staining with CD4, CD4^{IL-10} cells were fixed, permeabilized, and stained using the BD Cytofix/Cytoperm™ Kit according to the manufacturer's instructions (Cat. No. 554714, Biolegend, USA). Stained cells were washed two times with PBS supplemented with 1% FBS and analysed with a BD LSRFortessa analysed utilizing FlowJo 10 software.

Killing assays. After 2^{nd} and 3^{rd} re-stimulation, cytotoxicity of single-donor and polydonor CD4^{IL-10} cells was analysed in co-culture experiments (Locafaro et al. Mol Ther 2017, 25, 2254). Briefly, non-myeloid leukemia and a myeloid leukemia cell lines, K562 and ALL-CM respectively, were used as target cells and plated with CD4^{IL-10} cells at 1:1 ratio (105 target cells and 105 CD4^{IL-10} cells) for 3 days. At the end of co-culture, cells were harvested and K562 and ALL-CM cells were analysed based on CD45⁺, CD3—expression and counted by FACS.

Suppression assays. To measure the suppressive capacity of single donor and polydonor CD4^{IL-10} cells, allogeneic PBMC were labeled with Cell Proliferation Dye eFluor® 670 (Invitrogen, CA, USA), according to manufacturer's instructions. The labeled cells were activated with allogenic mature dendritic cells (DC) from CD14⁺ cells in the presence of GM-CSF and IL-4 in the presence an anti-CD3 mAb (50 ng/mL). Peripheral blood CD14 monocytes were positively selected using CD14 Microbeads (Miltenyi Biotec) according to the manufacturer's instructions. Cells were cultured in RPMI 1640 (Lonza) supplemented with 10% fetal bovine serum (FBS), 100 U/mL penicillin/streptomycin (Lonza), 2 mM L-glutamine (Lonza), at 37° C. in the presence of 10 ng/mL recombinant human (rh) IL-4 (R&D Systems) and 100 ng/mL recombinant human granulocyte-macrophage colony-stimulating factor (rhGM-CSF) (Genzyme) for 5 days. To generate mature dendritic cells (mDCs), on day 5 the cells were stimulated with 1 mg/mL lipopolysaccharide (LPS; Sigma) for an additional 2 days. At day 7, DCs were collected, phenotypically analyzed, and used to stimulate T cells. The purity and maturation state of DCs were checked by flow cytometry to determine expression of CD1a, CD14, CD86, CD83, and HLA-DR.

The labeled cells were plated in 96 well round well plates in final volumes of 200 μL and incubated for 3 days as follows: (i) Labeled PBMC alone 5×10⁴ cells/well; (ii) Labeled PBMC 5×10⁴ cells/well+mature DC 5×10³ cells/well+anti-CD3 mAb (50 ng/mL); (iii) Labeled PBMC 5×10⁴ cells/well+single or polydonor CD4^{IL-10} cells 5×10⁴ cells/well+mature DC 5×10³ cells/well+anti-CD3 mAb (50 ng/mL).

After 3 days of culture, the cells were harvested and transferred to 96 V bottom well plates for immunofluorescence staining. Cells were analyzed by FACS gated on living CD4+ eFluor670+ and CD8+eFluor670+ cells. Percentages of inhibition were calculated by measuring dilution of the eFluor670 label as described previously (Locafaro et al. Mol Ther 2017, 25, 2254).

Graft-versus Host Disease models: In all experiments 6/8 week-old female NSG mice were used. On day 0 mice received total body irradiation with a single dose of 175-200 cGy from a linear accelerator according to the weight of the mice. In some experiments mice received an single dose irradiation of 350 cGy. The mice were intravenously injected with PBMC cells (5×10⁶ or 2.5×10⁶), or CD4^{IL-10} cells (single-donors or polydonor—pool of three donors −5×10⁶ or 2.5×10⁶), or with PBMC (5×10⁶ or 2.5×10⁶) in combination with CD4^{IL-10} cells (5×10⁶ or 2.5×10⁶). Survival, weight loss, activity, fur, skin, and hunch were monitored at least 3 times per week as previously described (Bondanza et al. Blood. 2006; 107(5):1828-1836). Mice were euthanized for ethical reasons when their loss of bodyweight was 20%.

Alternatively, on day 0 mice received total body irradiation as above. On day 3 mice were injected with CD4⁺ T cells (2.5×10⁶), single and polydonor (pool of three donors) CD4^{IL-10} cells (2.5×10⁶), or CD4⁺ T cells (2.5×10⁶) in combination with single and polydonor (pool of three donors) CD4^{IL-10} cells (2.5×10⁶). GvHD induction was monitored as indicated above.

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

9. SEQUENCES

SEQ ID NO: 1 (Human IL-10 amino acid sequence--Protein Sequence: Ref P22301)
MHSSALLCCLVLLTGVRASPGQGTQSENSCTHFPGNLPNMLRDLRDAFSRVKTFFQMKDQLD
NLLLKESLLEDFKGYLGCQALSEMIQFYLEEVMPQAENQDPDIKAHVNSLGENLKTLRLRLR
RCHRFLPCENKSKAVEQVKNAFNKLQEKGIYKAMSEFDIFINYIEAYMIMKIRN
SEQ ID NO: 2 (Human IL-10 exemplary nucleic acid sequence)
atgcacagctcagcactgctctgttgcctggtcctcctgactggggtgagggccagcccagg
ccagggcacccagtctgagaacagctgcacccacttcccaggcaacctgcctaacatgcttc
gagatctccgagatgccttcagcagagtgaagactttctttcaaatgaaggatcagctggac
aacttgttgttaaaggagtccttgctggaggactttaagggttacctgggttgccaagcctt
gtctgagatgatccagttttacctggaggaggtgatgccccaagctgagaaccaagacccag
acatcaaggcgcatgtgaactccctgggggagaacctgaagaccctcaggctgaggctacgg
cgctgtcatcgatttcttccctgtgaaaacaagagcaaggccgtggagcaggtgaagaatgc
ctttaataagctccaagagaaaggcatctacaaagccatgagtgagtttgacatcttcatca
actacatagaagcctacatgacaatgaagatacgaaactga
SEQ ID NO: 3 (ANGFR amino acid sequence)
MGAGATGRAMDGPRLLLLLLLGVSLGGAKEACPTGLYTHSGECCKACNLGEGVAQPCGANQT
VCEPCLDSVTFSDVVSATEPCKPCTECVGLQSMSAPCVEADDAVCRCAYGYYQDETTGRCEA
CRVCEAGSGLVFSCQDKQNTVCEECPDGTYSDEANHVDPCLPCTVCEDTERQLRECTRWADA
ECEEIPGRWITRSTPPEGSDSTAPSTQEPEAPPEQDLIASTVAGVVTTVMGSSQPVVTRGTT
DNLIPVYCSILAAVVVGLVAYIAFKRWNRGIL
SEQ ID NO: 4 (ANGFR exemplary nucleic acid sequence )
atgggggcaggtgccaccggccgcgccatggacgggccgcgcctgctgctgttgctgcttct
gggggtgtcccttggaggtgccaaggaggcatgccccacaggcctgtacacacacagcggtg
agtgctgcaaagcctgcaacctgggcgagggtgtggcccagccttgtggagccaaccagacc
gtgtgtgagccctgcctggacagcgtgacgttctccgacgtggtgagcgcgaccgagccgtg
caagccgtgcaccgagtgcgtggggctccagagcatgtcggcgccgtgcgtggaggccgacg
acgccgtgtgccgctgcgcctacggctactaccaggatgagacgactgggcgctgcgaggcg
tgccgcgtgtgcgaggcgggctcgggcctcgtgttctcctgccaggacaagcagaacaccgt
gtgcgaggagtgccccgacggcacgtattccgacgaggccaaccacgtggacccgtgcctgc
cctgcaccgtgtgcgaggacaccgagcgccagctccgcgagtgcacacgctgggccgacgcc
gagtgcgaggagatccctggccgttggattacacggtccacacccccagagggctcggacag
cacagcccccagcacccaggagcctgaggcacctccagaacaagacctcatagccagcacgg
tggcaggtgtggtgaccacagtgatgggcagctcccagcccgtggtgacccgaggcaccacc
gacaacctcatccctgtctattgctccatcctggctgctgtggttgtgggccttgtggccta
catagccttcaagaggtggaacagggggatcctctag
SEQ ID NO: 5 (nucleotide sequence of pLVIL-10)
tggccattgcatacgttgtatccatatcataatatgtacatttatattggctcatgtccaac
attaccgccatgttgacattgattattgactagttattaatagtaatcaattacggggtcat
tagttcatagcccatatatggagttccgcgttacataacttacggtaaatggcccgcctggc
tgaccgcccaacgacccccgcccattgacgtcaataatgacgtatgttcccatagtaacgcc
aatagggactttccattgacgtcaatgggtggagtatttacggtaaactgcccacttggcag
tacatcaagtgtatcatatgccaagtacgccccctattgacgtcaatgacggtaaatggccc
gcctggcattatgcccagtacatgaccttatgggactttcctacttggcagtacatctacgt
attagtcatcgctattaccatggtgatgcggttttggcagtacatcaatgggcgtggatagc
ggtttgactcacggggatttccaagtctccaccccattgacgtcaatgggagtttgttttgg
caccaaaatcaacgggactttccaaaatgtcgtaacaactccgccccattgacgcaaatggg
cggtaggcgtgtacggtgggaggtctatataagcagagctcgtttagtgaaccggggtctct
ctggttagaccagatctgagcctgggagctctctggctaactagggaacccactgcttaagc
ctcaataaagcttgccttgagtgcttcaagtagtgtgtgcccgtctgttgtgtgactctggt
aactagagatccctcagacccttttagtcagtgtggaaaatctctagcagtggcgcccgaac
agggacttgaaagcgaaagggaaaccagaggagctctctcgacgcaggactcggcttgctga
agcgcgcacggcaagaggcgaggggcggcgactggtgagtacgccaaaaattttgactagcg
gaggctagaaggagagagatgggtgcgagagcgtcagtattaagcgggggagaattagatcg
cgatgggaaaaaattcggttaaggccagggggaaagaaaaaatataaattaaaacatatagt
atgggcaagcagggagctagaacgattcgcagttaatcctggcctgttagaaacatcagaag
gctgtagacaaatactgggacagctacaaccatcccttcagacaggatcagaagaacttaga
tcattatataatacagtagcaaccctctattgtgtgcatcaaaggatagagataaaagacac
caaggaagctttagacaagatagaggaagagcaaaacaaaagtaagaccaccgcacagcaag
cggccgctgatcttcagacctggaggaggagatatgagggacaattggagaagtgaattata
taaatataaagtagtaaaaattgaaccattaggagtagcacccaccaaggcaaagagaagag
tggtgcagagagaaaaaagagcagtgggaataggagctttgttccttgggttcttgggagca
gcaggaagcactatgggcgcagcgtcaatgacgctgacggtacaggccagacaattattgtc
tggtatagtgcagcagcagaacaatttgctgagggctattgaggcgcaacagcatctgttgc
aactcacagtctggggcatcaagcagctccaggcaagaatcctggctgtggaaagataccta
aaggatcaacagctcctggggatttggggttgctctggaaaactcatttgcaccactgctgt
gccttggaatgctagttggagtaataaatctctggaacagatttggaatcacacgacctgga
tggagtgggacagagaaattaacaattacacaagcttaatacactccttaattgaagaatcg
caaaaccagcaagaaaagaatgaacaagaattattggaattagataaatgggcaagtttgtg
gaattggtttaacataacaaattggctgtggtatataaaattattcataatgatagtaggag
gcttggtaggtttaagaatagtttttgctgtactttctatagtgaatagagttaggcaggga
tattcaccattatcgtttcagacccacctcccaaccccgaggggacccgacaggcccgaagg
aatagaagaagaaggtggagagagagacagagacagatccattcgattagtgaacggatctc
gacggtatcggttaacttttaaaagaaaaggggggattggggggtacagtgcaggggaaaga
atagtagacataatagcaacagacatacaaactaaagaattacaaaaacaaattacaaaaat
tcaaaattttatcgatcacgagactagcctcgagagatctgatcataatcagccataccaca
tttgtagaggttttacttgctttaaaaaacctcccacacctccccctgaacctgaaacataa
aatgaatgcaattgttgttgttaacttgtttattgcagcttataatggttacaaataaggca
atagcatcacaaatttcacaaataaggcatttttttcactgcattctagttttggtttgtcc
aaactcatcaatgtatcttatcatgtctggatctcaaatccctcggaagctgcgcctgtctt
aggttggagtgatacatttttatcacttttacccgtctttggattaggcagtagctctgacg
gccctcctgtcttaggttagtgaaaaatgtcactctcttacccgtcattggctgtccagctt
agctcgcaggggaggtggtctggatccaccatgtctagaggatccccctgttccacctcttg
aaggctatgtaggccacaaggcccacaaccacagcagccaggatggagcaatagacagggat
gaggttgtcggtggtgcctcgggtcaccacgggctgggagctgcccatcactgtggtcacca
cacctgccaccgtgctggctatgaggtcttgttctggaggtgcctcaggctcctgggtgctg
ggggctgtgctgtccgagccctctgggggtgtggaccgtgtaatccaacggccagggatctc
ctcgcactcggcgtcggcccagcgtgtgcactcgcggagctggcgctcggtgtcctcgcaca
cggtgcagggcaggcacgggtccacgtggttggcctcgtcggaatacgtgccgtcggggcac
tcctcgcacacggtgttctgcttgtcctggcaggagaacacgaggcccgagcccgcctcgca
cacgcggcacgcctcgcagcgcccagtcgtctcatcctggtagtagccgtaggcgcagcggc
acacggcgtcgtcggcctccacgcacggcgccgacatgctctggagccccacgcactcggtg
cacggcttgcacggctcggtcgcgctcaccacgtcggagaacgtcacgctgtccaggcaggg
ctcacacacggtctggttggctccacaaggctgggccacaccctcgcccaggttgcaggctt
tgcagcactcaccgctgtgtgtgtacaggcctgtggggcatgcctccttggcacctccaagg
gacacccccagaagcagcaacagcagcaggcgcggcccgtccatggcgcggccggtggcacc
tgcccccatcgcccgcctcccgcggcagcgctcgacttccagctcggtccgctttgcggact
gatggggctgcgctgcgctgcgctccagcgccccccctgcccgccggagctggccgcggccc
gaattcctgcaggaattcgatggaggctggatcggtcccggtgtcttctatggaggtcaaaa
cagcgtggatggcgtctccaggcgatctgacggttcactaaacgagctctgcttatataggc
ctcccaccgtacacgcctaccctcgagaagcttgatatcgaattcccacggggttggggttg
cgccttttccaaggcagccctgggtttgcgcagggacgcggctgctctgggcgtggttccgg
gaaacgcagcggcgccgaccctgggtctcgcacattcttcacgtccgttcgcagcgtcaccc
ggatcttcgccgctacccttgtgggccccccggcgacgcttcctgctccgcccctaagtcgg
gaaggttccttgcggttcgcggcgtgccggacgtgacaaacggaagccgcacgtctcactag
taccctcgcagacggacagcgccagggagcaatggcagcgcgccgaccgcgatgggctgtgg
ccaatagcggctgctcagcggggcgcgccgagagcagcggccgggaaggggcggtgcgggag
gcggggtgtggggcggtagtgtgggccctgttcctgcccgcgcggtgttccgcattctgcaa
gcctccggagcgcacgtcggcagtcggctccctcgttgaccgaatcaccgacctctctcccc
agggggatccccggtctgcaggaattcatgcacagctcagcactgctctgttgcctggtcct
cctgactggggtgagggccagcccaggccagggcacccagtctgagaacagctgcacccact
tcccaggcaacctgcctaacatgcttcgagatctccgagatgccttcagcagagtgaagact
ttctttcaaatgaaggatcagctggacaacttgttgttaaaggagtccttgctggaggactt
taagggttacctgggttgccaagccttgtctgagatgatccagttttacctggaggaggtga
tgccccaagctgagaaccaagacccagacatcaaggcgcatgtgaactccctgggggagaac
ctgaagaccctcaggctgaggctacggcgctgtcatcgatttcttccctgtgaaaacaagag
caaggccgtggagcaggtgaagaatgcctttaataagctccaagagaaaggcatctacaaag
ccatgagtgagtttgacatcttcatcaactacatagaagcctacatgacaatgaagatacga
aactgagtcgagaatcaacctctggattacaaaatttgtgaaagattgactggtattcttaa
ctatgttgctccttttacgctatgtggatacgctgctttaatgcctttgtatcatgctattg
cttcccgtatggctttcattttctcctccttgtataaatcctggttgctgtctctttatgag
gagttgtggcccgttgtcaggcaacgtggcgtggtgtgcactgtgtttgctgacgcaacccc
cactggttggggcattgccaccacctgtcagctcctttccgggactttcgctttccccctcc
ctattgccacggcggaactcatcgccgcctgccttgcccgctgctggacaggggctcggctg
ttgggcactgacaattccgtggtgttgtcggggaaatcatcgtcctttccttggctgctcgc
ctgtgttgccacctggattctgcgcgggacgtccttctgctacgtcccttcggccctcaatc
cagcggaccttccttcccgcggcctgctgccggctctgcggcctcttccgcgtcttcgcctt
cgccctcagacgagtcggatctccctttgggccgcctccccgcctggaattcgagctcggta
cctttaagaccaatgacttacaaggcagctgtagatcttagccactttttaaaagaaaaggg
gggactggaagggctaattcactcccaacgaagacaagatctgctttttgcttgtactgggt
ctctctggttagaccagatctgagcctgggagctctctggctaactagggaacccactgctt
aagcctcaataaagcttgccttgagtgcttcaagtagtgtgtgcccgtctgttgtgtgactc
tggtaactagagatccctcagacccttttagtcagtgtggaaaatctctagcagtagtagtt
catgtcatcttattattcagtatttataacttgcaaagaaatgaatatcagagagtgagagg
aacttgtttattgcagcttataatggttacaaataaagcaatagcatcacaaatttcacaaa
taaagcatttttttcactgcattctagttgtggtttgtccaaactcatcaatgtatcttatc
atgtctggctctagctatcccgcccctaactccgcccatcccgcccctaactccgcccagtt
ccgcccattctccgccccatggctgactaattttttttatttatgcagaggccgaggccgcc
tcggcctctgagctattccagaagtagtgaggaggcttttttggaggcctaggcttttgcgt
cgagacgtacccaattcgccctatagtgagtcgtattacgcgcgctcactggccgtcgtttt
acaacgtcgtgactgggaaaaccctggcgttacccaacttaatcgccttgcagcacatcccc
ctttcgccagctggcgtaatagcgaagaggcccgcaccgatcgcccttcccaagcggccgca
cgctcagtggaacgaaaactcacgttaagggattttggtcatgaacaataaaactgtctgct
tacataaacagtaatacaaggggtgttatgagccatattcaacgggaaacgtcttgctctag
gccgcgattaaattccaacatggatgctgatttatatgggtataaatgggctcgcgataatg
tcgggcaatcaggtgcgacaatctatcgattgtatgggaagcccgatgcgccagagttgttt
ctgaaacatggcaaaggtagcgttgccaatgatgttacagatgagatggtcagactaaactg
gctgacggaatttatgcctcttccgaccatcaagcattttatccgtactcctgatgatgcat
ggttactcaccactgcgatccccgggaaaacagcattccaggtattagaagaatatcctgat
tcaggtgaaaacattgttgatgcgctggcagtgttcctgcgccggttgcattcgattcctgt
ttgtaattgtccttttaacagcgatcgcgtatttcgtctcgctcaggcgcaatcacgaatga
ataacggtttggttgatgcgagtgattttgatgacgagcgtaatggctggcctgttgaacaa
gtctggaaagaaatgcataaacttttgccattctcaccggattcagtcgtcactcatggtga
tttctcacttgataaccttatttttgacgaggggaaattaataggttgtattgatgttggac
gagtcggaatcgcagaccgataccaggatcttgccatcctatggaactgcctcggtgagttt
tctccttcattacagaaacggctttttcaaaaatatggtattgataatcctgatatgaataa
attgcagtttcatttgatgctcgatgagtttttctaagaattaattcatgagcggatacata
tttgaatgtatttagaaaaataaacaaataggggttccgcgactatgtctttgataatctca
tgaccaaaatcccttaacgtgagttttcgttccactgagcgtcagaccccgtagaaaagatc
aaaggatcttcttgagatcctttttttctgcgcgtaatctgctgcttgcaaacaaaaaaacc
accgctaccagcggtggtttgtttgccggatcaagagctaccaactctttttccgaaggtaa
ctggcttcagcagagcgcagataccaaatactgtccttctagtgtagccgtagttaggccac
cacttcaagaactctgtagcaccgcctacatacctcgctctgctaatcctgttaccagtggc
tgctgccagtggcgataagtcgtgtcttaccgggttggactcaagacgatagttaccggata
aggcgcagcggtcgggctgaacggggggttcgtgcacacagcccagcttggagcgaacgacc
tacaccgaactgagatacctacagcgtgagctatgagaaagcgccacgcttcccgaagggag
aaaggcggacaggtatccggtaagcggcagggtcggaacaggagagcgcacgagggagcttc
cagggggaaacgcctggtatctttatagtcctgtcgggtttcgccacctctgacttgagcgt
cgatttttgtgatgctcgtcaggggggcggagcctatggaaaaacgccagcaacgcggcctt
tttacggttcctggccttttgctggcgttatcccctgattctgtggataaccgtattaccgc
ctttgagtgagctgataccgctcgccgcagccgaacgaccgagcgcagcgagtcagtgagcg
aggaagcggaagagcgcccaatacgcaaaccgcctctccccgcgcgttgtatgcttccggct
cgtatgttgtgtggaattgtgagcggataacaatttcacacaggaaacagctatgaccatga
ttacgccaagccgaattaaccctcactaaagggaacagctagc
SEQ ID NO: 6 (Viral interleukin-10 homolog aka interleukin-10 BCRF1 aka
IL10H_EBVB9) Protein Sequence: Ref: P03180
MERRLVVTLQCLVLLYLAPECGGTDQCDNFPQMLRDLRDAFSRVKTFFQTKDEVDNLLLKES
LLEDFKGYLGCQALSEMIQFYLEEVMPQAENQDPEAKDHVNSLGENLKTLRLRLRRCHRFLP
CENKSKAVEQIKNAFNKLQEKGIYKAMSEFDIFINYIEAYMTIKAR*
SEQ ID NO: 7 (Viral interleukin-10 homolog cDNA sequence)
Nucleotide sequence (cDNA): Ref: NC_007605.1
5′_atggagcgaaggttagtggtcactctgcagtgcctggtgctgctttacctggcacctga
gtgtggaggtacagaccaatgtgacaattttccccaaatgttgagggacctaagagatgcct
tcagtcgtgttaaaacctttttccagacaaaggacgaggtagataaccttttgctcaaggag
tctctgctagaggactttaagggctaccttggatgccaggccctgtcagaaatgatccaatt
ctacctggaggaagtcatgccacaggctgaaaaccaggaccctgaagccaaagaccatgtca
attctttgggtgaaaatctaaagaccctacggctccgcctgcgcaggtgccacaggttcctg
ccgtgtgagaacaagagtaaagctgtggaacagataaaaaatgcctttaacaagctgcagga
aaaaggaatttacaaagccatgagtgaatttgacatttttattaactacatagaagcataca
tgacaattaaagccaggtga_3′
SEQ ID NO: 8 (exemplary human IL-10 variant with amino acid substitutions based
on viral IL-10)
MHSSALLCCLVLLTGVRASPGQGTQSENSCTHFPGNLPNMLRDLRDAFSRVKTFFQTKDEVD
NLLLKESLLEDFKGYLGCQALSEMIQFYLEEVMPQAENQDPEAKDHVNSLGENLKTLRLRLR
RCHRFLPCENKSKAVEQIKNAFNKLQEKGIYKAMSEFDIFINYIEAYMTMKIRN
SEQ ID NO: 9 (exemplary human IL-10 variant with amino acid substitutions based
on viral IL-10)
MHSSALLCCLVLLTGVRASPGQGTQSENSCTHFPGNLPNMLRDLRDAFSRVKTFFQMKDQLD
NLLLKESLLEDFKGYLGCQALSEMIQFYLEEVMPQAENQDPDAKAHVNSLGENLKTLRLRLR
RCHRFLPCENKSKAVEQVKNAFNKLQEKGIYKAMSEFDIFINYIEAYMTMKIRN
SEQ ID NO: 10 (Mus musculus; “MOUSE”)
MPGSALLCCLLLLTGMRISRGQYSREDNNCTHFPVGQSHMLLELRTAFSQVKTFFQTKDQLD
NILLTDSLMQDFKGYLGCQALSEMIQFYLVEVMPQAEKHGPEIKEHLNSLGEKLKTLRMRLR
RCHRFLPCENKSKAVEQVKSDFNKLQDQGVYKAMNEFDIFINCIEAYMMIKMKS
SEQ ID NO: 11 (Rattus norvegicus; “RAT”)
MPGSALLCCLLLLAGVKTSKGHSIRGDNNCTHFPVSQTHMLRELRAAFSQVKTFFQKKDQLD
NILLTDSLLQDFKGYLGCQALSEMIKFYLVEVMPQAENHGPEIKEHLNSLGEKLKTLWIQLR
RCHRFLPCENKSKAVEQVKNDFNKLQDKGVYKAMNEFDIFINCIEAYVTLKMKN
SEQ ID NO: 12 (Macaca mulatta; “MACMU”)
MHSSALLCCLVLLTGVRASPGQGTQSENSCTRFPGNLPHMLRDLRDAFSRVKTFFQMKDQLD
NILLKESLLEDFKGYLGCQALSEMIQFYLEEVMPQAENHDPDIKEHVNSLGENLKTLRLRLR
RCHRFLPCENKSKAVEQVKNAFSKLQEKGVYKAMSEFDIFINYI EAYMTMKIQN
SEQ ID NO: 13 (Gorilla gorilla; “GORILLA”)
MHSSALLCCLVLLTGVRASPGHGTQSENSCTHFPGNLPNMLRDLRDAFSRVKTFFQMKDQLD
NLLLKESLLEDFKGYLGCQALSEMIQFYLEEVMPQAENQDPDIKAHVNSLGENLKTLRLRLR
RCHRFLPCENKSKAVEQVKNAFNKLQEKGVYKAMSEFDIFINYIEAYMTMKIRN
SEQ ID NO: 14 (Macaca fascicularis; “CYNO”)
MHSSALLCCLVLLTGVRASPGQGTQSENSCTRFPGNLPHMLRDLRDAFSRVKTFFQMKDQLD
NILLKESLLEDFKGYLGCQALSEMIQFYLEEVMPQAENHDPDIKEHVNSLGENLKTLRLRLR
RCHRFLPCENKSKAVEQVKNAFSKLQEKGVYKAMSEFDIFINYIEAYMTMKIQN
SEQ ID NO: 15 (Papio Anubis; “OLIVE BABOON”)
MHSSALLCCLVVLTGVRASPGQGTQSENSCTRFPGNLPHMLRDLRDAFSRVKTFFQMKDQLD
NILLKESLLEDFKGYLGCQALSEMIQFYLEEVMPQAENHDPDIKEHVNSLGENLKTLRLRLR
RCHRFLPCENKSKAVEQVKNAFSKLQEKGVYKAMSEFDIFINYIEAYMTMKIQN
SEQ ID NO: 16 (Pan paniscus; “BONOBO”)
MHSSALLCCLVLLTGVRASPGQGTQSENSCTHFPGNLPNMLRDLRDAFSRVKTFFQMKDQLD
NLLLKESLLEDFKGYLGCQALSEMIQFYLEEVMPQAENQDPDIKVHVNSLGENLKTLRLRLR
RCHRFLPCENKSKAVEQVKNAFNKLQEKGIYKAMSEFDIFINYIEAYMTMKIRN
SEQ ID NO: 17 (Pan troglodytes; “CHIMP”)
MHSSALLCCLVLLTGVRASPGQGTQSENSCTHFPGNLPNMLRDLRDAFSRVKTFFQMKDQLD
NLLLKESLLEDFKGYLGCQALXEMIQFYLEEVMPQAENQDPDIKVHVNSLGENLKTLRLRLR
RCHRFLPCENKSKAVEQVKNAFNKLQEKGIVKAMSEFDIFINYIEAYMTMKIRN
SEQ ID NO: 18 (EBVB9)
MERRLVVTLQCLVLLYLAPECGGTDQCDNFPQMLRDLRDAFSRVKTFFQTKDEVDNLLLKES
LLEDFKGYLGCQALSEMIQFYLEEVMPQAENQDPEAKDHVNSLGENLKTLRLRLRRCHRFLP
CENKSKAVEQIKNAFNKLQEKGIYKAMSEFDIFINYIEAYMTIKAR
SEQ ID NO: 19 huIL-10 HYBRID#1
MHSSALLCCLVLLTGRASPGQGTQSENSCTHFPGNIPNMLRDIRDAFSRVKTEFQTKDEVDN
LLLKESLLEDEKGYLGCQALSEMIQFYLEEVMPQAENQDPEAKDHVNSLGENLKTLRLRIRR
CHRFLPCENKSKAVEQIKNAFNKLQEKGIYKAMSEFDIFINYIEAYMTMKIRN
SEQ ID NO: 20 huIL-10 HYBRID#2
MHSSALLCCLVLLTGRASPGQGTQSENSCTHFPGNIPNMLRDIRDAFSRVKTEFQMKDQLDN
LLLKESLLEDEKGYLGCQALSEMIQFYLEEVMPQAENQDPDAKAHVNSLGENLKTLRLRIRR
CHRFLPCENKSKAVEQVKNAFNKLQEKGIYKAMSEFDIFINYIEAYMTMKIRN

What is claimed is:

1. A population of CD4⁺ T cells (polydonor CD4^{IL-10} cells) that have been genetically modified to comprise an exogenous polynucleotide encoding IL-10, wherein the CD4⁺ T cells were obtained from at least two different T cell donors.

2. The population of CD4⁺ T cells of claim 1, wherein the CD4⁺ T cells were obtained from two, three, four, five, six, seven, eight, nine, or ten different T cell donors.

3. The population of CD4⁺ T cells of claim 1 or claim 2, wherein the CD4⁺ T cells in the population collectively have six, seven, eight, nine, ten, eleven, twelve, or more different HLA haplotypes.

4. The population of CD4⁺ T cells of any one of the preceding claims, wherein all the CD4⁺ T cells in the population have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other.

5. The population of CD4⁺ T cells of any one of the preceding claims, wherein all the CD4⁺ T cells in the population have at least 1/8, 2/8, 3/8, 4/8, 5/8, 6/8, 7/8, or 8/8 match at the HLA-A, HLA-B, HLA-C, and HLA-DRB1 loci to each other.

6. The population of CD4⁺ T cells of any one of the preceding claims, wherein all the CD4⁺ T cells in the population have 2/2 match at the HLA-A locus to each other.

7. The population of CD4⁺ T cells of any one of the preceding claims, wherein all the CD4⁺ T cell in the population have 2/2 match at the HLA-B locus to each other.

8. The population of CD4⁺ T cells of any one of the preceding claims, wherein all the CD4⁺ T cell in the population have 2/2 match at the HLA-C locus to each other.

9. The population of CD4⁺ T cells of any one of the preceding claims, wherein all the CD4⁺ T cells in the population have at least 3/4 or 4/4 match at the HLA-DRB1 and HLA-DQB1 loci with each other.

10. The population of CD4⁺ T cells of any one of claims 1-3, wherein all the CD4⁺ T cells in the population have less than 5/10, 6/10, 7/10, 8/10, or 9/10 match at the HLA-A, HLA-B, HLA-C, HLA-DRB1, and HLA-DQB1 loci to each other.

11. The population of CD4⁺ T cells of claim 10, wherein all the CD4⁺ T cells in the population have less than 4/8, 5/8, 6/8, 7/8, or 8/8 match at the HLA-A, HLA-B, HLA-C, and HLA-DRB1 loci to each other.

12. The population of CD4⁺ T cells of any one of claims 10-11, wherein all the CD4⁺ T cells in the population have less than 2/2 match at the HLA-A locus to each other.

13. The population of CD4⁺ T cells of any one of claims 10-12, wherein all the CD4⁺ T cell in the population have less than 2/2 match at the HLA-B locus to each other.

14. The population of CD4⁺ T cells of any one of claims 10-13, wherein all the CD4⁺ T cell in the population have less than 2/2 match at the HLA-C locus to each other.

15. The population of CD4⁺ T cells of any one of claims 10-14, wherein all the CD4⁺ T cells in the population have less than 2/4, 3/4 or 4/4 match at the HLA-DRB1 and HLA-DQB1 loci with each other.

16. The population of CD4⁺ T cells of any one of the preceding claims, wherein all the CD4⁺ T cells in the population have an A*02 or A*24 allele.

17. The population of CD4⁺ T cells of any one of the preceding claims, wherein none of the CD4⁺ T cells is immortalized.

18. The population of CD4⁺ T cells of any one of the preceding claims, wherein the exogenous polynucleotide comprises an IL-10-encoding polynucleotide segment operably linked to expression control elements.

19. The population of CD4⁺ T cells of any one of the preceding claims, wherein the IL-10 is a human IL-10.

20. The population of CD4⁺ T cells of any one of claims 1-19, wherein the IL-10 is a viral IL-10.

21. The population of CD4⁺ T cells of any one of the preceding claims, wherein the IL-10-encoding polynucleotide segment encodes a protein having the sequence of SEQ ID NO:1.

22. The population of CD4⁺ T cells of claim 21, wherein the IL-10-encoding polynucleotide segment has the sequence of SEQ ID NO:2.

23. The population of CD4⁺ T cells of claim 20, wherein the IL-10-encoding polynucleotide segment encodes a protein having the sequence of SEQ ID NO: 6.

24. The population of CD4⁺ T cells of claim 23, wherein the IL-10-encoding polynucleotide segment has the sequence of SEQ ID NO: 7.

25. The population of CD4⁺ T cells of any one of claims 18-24, wherein the expression control elements drive constitutive expression of the encoded IL-10.

26. The population of CD4⁺ T cells of any one of the preceding claims, wherein the exogenous polynucleotide further comprises a polynucleotide segment encoding a selection marker.

27. The population of CD4⁺ T cells of claim 26, wherein the selection marker is ΔNGFR.

28. The population of CD4⁺ T cells of claim 27, wherein the ΔNGFR has the sequence of SEQ ID NO: 3.

29. The population of CD4⁺ T cells of claim 27, wherein the polynucleotide segment comprises a sequence of SEQ ID NO:4.

30. The population of CD4⁺ T cells of claim 26, wherein the selection marker is a truncated form of EGFR polypeptide.

31. The population of CD4⁺ T cells of any one of the preceding claims, wherein the exogenous polynucleotide having a sequence of SEQ ID NO: 5.

32. The population of CD4⁺ T cells of any one of claims 1-31, wherein the exogenous polynucleotide is integrated into the T cell nuclear genome.

33. The population of CD4⁺ T cells of any one of claims 1-31, wherein the exogenous polynucleotide is not integrated into the T cell nuclear genome.

34. The population of CD4⁺ T cells of claim 32 or 33, wherein the exogenous polynucleotide further comprises lentiviral vector sequences.

35. The population of CD4⁺ T cells of any one of claims 1-34, wherein the exogenous polynucleotide is not integrated into the T cell nuclear genome.

36. The population of CD4⁺ T cells of any one of the preceding claims, wherein at least 70% of the CD4⁺ T cells within the population express IL-10.

37. The population of CD4⁺ T cells of claim 36, wherein at least 90% of the CD4⁺ T cells within the population express IL-10.

38. The population of CD4⁺ T cells of claim 37, wherein at least 95% or 98% of the CD4⁺ T cells within the population express IL-10.

39. The population of CD4⁺ T cells of any one of the preceding claims, wherein the genetically modified CD4⁺ T cells constitutively express at least 100 pg IL-10 per 10⁶ of the CD4⁺ T cells/mL of culture medium.

40. The population of CD4⁺ T cells of claim 39, wherein the genetically modified CD4⁺ T cells constitutively express at least 100 pg, 200 pg, 500 pg, 1 ng, 5 ng, 10 ng, or 50 ng IL-10 per 10⁶ of the CD4⁺ T cells/mL.

41. The population of CD4⁺ T cells of any one of the preceding claims, wherein the genetically modified CD4⁺ T cells express at least 1 ng IL-10 per 10⁶ of the CD4⁺ T cells/mL after activation with anti-CD3 and anti-CD28 antibodies.

42. The population of CD4⁺ T cells of claim 41, wherein the genetically modified CD4⁺ T cells express at least 2 ng, 5 ng, 10 ng, 100 ng, 200 ng, or 500 ng IL-10 per 10⁶ of the CD4⁺ T cells/mL after activation with anti-CD3 and anti-CD28 antibodies.

43. The population of CD4⁺ T cells of any one of the preceding claims, wherein the genetically modified CD4⁺ T cells express IL-10 at a level at least 5-fold higher than unmodified CD4⁺ T cells.

44. The population of CD4⁺ T cells of claim 43, wherein the genetically modified CD4⁺ T cells express IL-10 at a level at least 10-fold higher than unmodified CD4⁺ T cells.

45. The population of CD4⁺ T cells of any one of the preceding claims, wherein at least 70% of the CD4⁺ T cells within the population express the selection marker from the exogenous polynucleotide.

46. The population of CD4⁺ T cells of claim 45, wherein at least 90% of the CD4⁺ T cells within the population express the selection marker from the exogenous polynucleotide.

47. The population of CD4⁺ T cells of claim 46, wherein at least 95% or 98% of the CD4⁺ T cells within the population express the selection marker from the exogenous polynucleotide.

48. The population of CD4⁺ T cells of any one of the preceding claims, wherein the genetically modified CD4⁺ T cells express CD49b.

49. The population of CD4⁺ T cells of any one of the preceding claims, wherein the genetically modified CD4⁺ T cells express LAG-3.

50. The population of CD4⁺ T cells of any one of the preceding claims, wherein the genetically modified CD4⁺ T cells express TGF-β.

51. The population of CD4⁺ T cells of any one of the preceding claims, wherein the genetically modified CD4⁺ T cells express IFN-γ.

52. The population of CD4⁺ T cells of any one of the preceding claims, wherein the genetically modified CD4⁺ T cells express GzB.

53. The population of CD4⁺ T cells of any one of the preceding claims, wherein the genetically modified CD4⁺ T cells express perforin.

54. The population of CD4⁺ T cells of any one of the preceding claims, wherein the genetically modified CD4⁺ T cells express CD18.

55. The population of CD4⁺ T cells of any one of the preceding claims, wherein the genetically modified CD4⁺ T cells express CD2.

56. The population of CD4⁺ T cells of any one of the preceding claims, wherein the genetically modified CD4⁺ T cells express CD226.

57. The population of CD4⁺ T cells of any one of the preceding claims, wherein the genetically modified CD4⁺ T cells express IL-22.

58. The population of CD4⁺ T cells of any one of the preceding claims, wherein the CD4⁺ T cells have not been anergized in the presence of peripheral blood mononuclear cells (PBMCs) from a host.

59. The population of CD4⁺ T cells of any one of the preceding claims, wherein the CD4⁺ T cells have not been anergized in the presence of recombinant IL-10 protein, wherein the recombinant IL-10 protein is not expressed from the CD4⁺ T cells.

60. The population of CD4⁺ T cells of any one of the preceding claims, wherein the CD4⁺ T cells have not been anergized in the presence of DC10 cells from a host.

61. The population of CD4⁺ T cells of any one of claims 1-60, wherein the CD4⁺ T cells are in a frozen suspension.

62. The population of CD4⁺ T cells of any one of claims 1-60, wherein the CD4⁺ T cells are in a liquid suspension.

63. The population of CD4⁺ T cells of claim 62, wherein the liquid suspension has previously been frozen.

64. A pharmaceutical composition comprising:
(i) the population of CD4⁺ T cells of any one of the preceding claims; suspended in
(ii) a pharmaceutically acceptable carrier.

65. A method of making polydonor CD4^{IL-10} cells, comprising the steps of:
(i) pooling primary CD4⁺ T cells obtained from at least two different T cell donors; and
(ii) modifying the pooled CD4⁺ T cells by introducing an exogenous polynucleotide encoding IL-10,
thereby obtaining the polydonor CD4^{IL-10} cells.

66. A method of making polydonor CD4^{IL-10} cells, comprising the steps of:
(i) obtaining primary CD4⁺ T cells from at least two different T cell donors; and
(ii) separately modifying each donor's CD4⁺ T cells by introducing an exogenous polynucleotide encoding IL-10, and then
(iii) pooling the genetically modified CD4⁺ T cells,
thereby obtaining the polydonor CD4^{IL-10} cells.

67. The method of claim 65 or claim 66, further comprising the step, after step (i) and before step (ii), after step (ii), after step (ii) and before step (iii), or after step (iii) of:
incubating the primary CD4⁺ T cells in the presence of an anti-CD3 antibody, and anti-CD28 antibody or anti-CD3 antibody and CD28 antibody coated beads.

68. The method of claim 67, wherein the primary CD4⁺ T cells are further incubated in the presence of IL-2.

69. The method of any one of claims 65-68, wherein the exogenous polynucleotide is introduced into the primary CD4⁺ T cells using a viral vector.

70. The method of claim 69, wherein the viral vector is a lentiviral vector.

71. The method of any one of claims 65-70, wherein the exogenous polynucleotide comprises a segment encoding IL-10 having the sequence of SEQ ID NO:1.

72. The method of any one of claims 65-70, wherein the IL-10-encoding polynucleotide segment has the sequence of SEQ ID NO:2 or 7.

73. The method of any one of claims 65-72, wherein the exogenous polynucleotide further comprises a segment encoding a selection marker.

74. The method of claim 73, wherein the encoded selection marker is ΔNGFR.

75. The method of claim 74, wherein the encoded selection marker has the sequence of SEQ ID NO:3.

76. The method of any one of claims 73-75, further comprising the step, after step (ii), of:
isolating the genetically-modified CD4⁺ T cells expressing the selection marker, thereby generating an enriched population of genetically-modified CD4⁺ T cells.

77. The method of claim 76, wherein at least 70% of the genetically-modified CD4⁺ T cells in the enriched population express IL-10.

78. The method of claim 77, wherein at least 90%, 95% or 98% of the genetically-modified CD4⁺ T cells in the enriched population express IL-10.

79. The method of any one of claims 76-69, wherein at least 70% of the genetically-modified CD4⁺ T cells in the enriched population express the selection marker.

80. The method of claim 70, wherein at least 90%, 95%, or 98% of the genetically-modified CD4⁺ T cells in the enriched population express the selection marker.

81. The method of any one of claims 76-80, further comprising the step of incubating the enriched population of genetically-modified CD4⁺ T cells.

82. The method of claim 81, wherein the step of incubating the enriched population of genetically-modified CD4⁺ T cells is performed in the presence of anti-CD3 antibody and anti-CD28 antibody or CD3 antibody and CD28 antibody coated beads in the presence of IL-2.

83. The method of any one of claims 65-82, further comprising the later step of freezing the genetically-modified CD4⁺ T cells.

84. The method of any one of claims 65-83, wherein in step (i), the primary CD4⁺ T cells are obtained from two, three, four, five, six, seven, eight, nine, or ten different T cell donors.

85. The method of any one of claim 84, wherein the at least two T cell donors have at least 1/10, 2/10, 3/10, 4/10, 5/10, 6/10, 7/10, 8/10, or 9/10 match at the HLA-A, HLA-B, HLA-C, HLA-DRB1, and HLA-DQB1 loci to each other.

86. The method of any one of claims 65-85, wherein the at least two T cell donors have at least 1/8, 2/8, 3/8, 4/8, 5/8, 6/8, 7/8, or 8/8 match at the HLA-A, HLA-B, HLA-C, and HLA-DRB1 loci to each other.

87. The method of any one of claims 65-86, wherein the at least two T cell donors have 2/2 match at the HLA-A locus to each other.

88. The method of any one of claims 65-87, wherein the at least two T cell donors have 2/2 match at the HLA-B locus to each other.

89. The method of any one of claims 65-88, wherein the at least two T cell donors have 2/2 match at the HLA-C locus to each other.

90. The method of any one of claims 65-89, wherein the at least two T cell donors have at least 3/4 or 4/4 match at the HLA-DRB1 and HLA-DQB1 loci to each other.

91. The method of any one of claim 84, wherein the at least two T cell donors have less than 5/10, 6/10, 7/10, 8/10, or 9/10 match at the HLA-A, HLA-B, HLA-C, HLA-DRB1, and HLA-DQB1 loci to each other.

92. The method of claim 84 or 91, wherein the at least two T cell donors have less than 4/8, 5/8, 6/8, 7/8, or 8/8 match at the HLA-A, HLA-B, HLA-C, and HLA-DRB1 loci to each other.

93. The method of any one of claims 84 and 91-92, wherein the at least two T cell donors have less than 2/2 match at the HLA-A locus to each other.

94. The method of any one of claims 84 and 91-93, wherein the at least two T cell donors have less than 2/2 match at the HLA-B locus to each other.

95. The method of any one of claims 84 and 91-94, wherein the at least two T cell donors have less than 2/2 match at the HLA-C locus to each other.

96. The method of any one of claims 84 and 91-95, wherein the at least two T cell donors have less than 3/4 or 4/4 match at the HLA-DRB1 and HLA-DQB1 loci to each other.

97. The method of any one of claims 65-96, wherein each of the at least two T cell donors has an A*02 or A*24 allele.

98. The method of any one of claims 65-97, wherein in step (i), the primary CD4⁺ T cells are obtained from one or more frozen stocks.

99. The method of any one of claims 65-97, wherein in step (i), the primary CD4⁺ T cells are obtained from unfrozen peripheral blood mononuclear cells of the at least two different T cell donors.

100. The method of claim 99, further comprising the step of isolating CD4⁺ T cells from the peripheral blood mononuclear cells.

101. A method of treating a patient, comprising:
administering the polydonor CD4^{IL-10} cells of any one of claims 1-63, or the pharmaceutical composition of claim 64, to a patient in need of immune tolerization.

102. The method of claim 101, further comprising the preceding step of thawing a frozen suspension of polydonor CD4^{IL-10} cells.

103. The method of claim 101 or 102, wherein the polydonor CD4^{IL-10} cells or the pharmaceutical composition prevents or reduces severity of pathogenic T cell response in the patient.

104. The method of claim 101 or 102, wherein the polydonor CD4^{IL-10} cells or the pharmaceutical composition reduces inflammation or enhances immunological tolerance.

105. The method of claim 101 or 102, wherein the polydonor CD4^{IL-10} cells or the pharmaceutical composition enhances tissue repair.

106. The method of any one of claims 101-105, further comprising the step of administering mononuclear cells to the patient.

107. The method of claim 106, wherein the polydonor CD4^{IL-10} cells or the pharmaceutical composition and the mononuclear cells are administered concurrently.

108. The method of claim 106, wherein the mononuclear cells are administered either prior to or subsequent to administration of the polydonor CD4^{IL-10} cells or the pharmaceutical composition.

109. The method of any one of claims 101-108, further comprising the step of:
administering hematopoietic stem cells (HSC) of an HSC donor to the patient either prior to or subsequent to administration of the polydonor CD4^{IL-10} cells or pharmaceutical composition.

110. The method of claim 109, wherein the HSC donor is partially HLA-mismatched to the patient.

111. The method of claim 110, wherein the HSC donor has less than 5/10, 6/10, 7/10, 8/10, 9/10 or 10/10 match at the HLA-A, HLA-B, HLA-C, HLA-DRB1, and HLA-DQB1 loci to the patient.

112. The method of claim 110, wherein the HSC donor has less than 4/8, 5/8, 6/8, 7/8, or 8/8 match at the HLA-A, HLA-B, HLA-C, and HLA-DRB1 loci to the patient.

113. The method of claim 110, wherein the HSC donor has less than 2/2 match at the HLA-A, HLA-B, or HLA-C locus to the patient.

114. The method of claim 110, wherein the HSC donor has less than 3/4 or 4/4 match at the HLA-DRB1 and HLA-DQB1 loci to the patient.

115. The method of any one of claims 101-114, wherein one or more of the T cell donors are HLA-mismatched or partially HLA-mismatched to the patient.

116. The method of claim 115, wherein one or more of the T cell donors have less than 5/10, 6/10, 7/10, 8/10, 9/10 or 10/10 match at the HLA-A, HLA-B, HLA-C, HLA-DRB1, and HLA-DQB1 loci to the patient.

117. The method of claim 115, wherein one or more of the T cell donors have less than 4/8, 5/8, 6/8, 7/8, or 8/8 match at the HLA-A, HLA-B, HLA-C, and HLA-DRB1 loci to the patient.

118. The method of claim 115, wherein one or more of the T cell donors have less than 2/2 match at the HLA-A, HLA-B, or HLA-C locus to the patient.

119. The method of claim 115, wherein one or more of the T cell donors have less than 2/4, 3/4 or 4/4 match at the HLA-DRB1 and HLA-DQB1 loci to the patient.

120. The method of any one of claims 101-119, wherein one or more of the T cell donors are HLA-mismatched or partially HLA-mismatched with the HSC donor.

121. The method of claim 120, wherein one or more of the T cell donors have less than 5/10, 6/10, 7/10, 8/10, 9/10 or 10/10 match at the HLA-A, HLA-B, HLA-C, HLA-DRB1, and HLA-DQB1 loci to the HSC donor.

122. The method of claim 120, wherein one or more of the T cell donors have less than 4/8, 5/8, 6/8, 7/8, or 8/8 match at the HLA-A, HLA-B, HLA-C, and HLA-DRB1 loci to the HSC donor.

123. The method of claim 120, wherein one or more of the T cell donors have less than 2/2 match at the HLA-A, HLA-B, or HLA-C locus to the HSC donor.

124. The method of claim 120, wherein one or more of the T cell donors have less than 3/4 or 4/4 match at the HLA-DRB1 and HLA-DQB1 loci to the HSC donor.

125. The method of any one of claims 101-124, wherein the polydonor CD4^{IL-10} cells or the pharmaceutical composition prevents or reduces severity of GvHD by the transplanted hematopoietic stem cells.

126. The method of any one of claims 109-125, wherein the polydonor CD4^{IL-10} cells or the pharmaceutical composition prevents or reduces severity of pathogenic response of lymphoid cells from the transplanted hematopoietic cells.

127. The method of any one of claims 101-126, wherein the patient has neoplastic cells.

128. The method of claim 127, wherein the neoplastic cells express CD13, HLA-class I and CD54.

129. The method of any one of claims 127-128, wherein the neoplastic cells express CD112, CD58, or CD155.

130. The method of any one of claims 127-129, wherein the patient has a cancer, optionally wherein the cancer is a solid or hematological neoplasm.

131. The method of any one of claims 101-130, wherein the patient has a cancer selected from the group consisting of: Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Acute Lymphocytic (ALL), Acute Myeloid (AML, including myeloid sarcoma and leukemia cutis), Chronic Lymphocytic (CLL), Chronic Myeloid (CML) Leukemia, Chronic Myelomonocytic (CMML), Leukemia in Children, Liver Cancer, Lung Cancer, Lung Cancer with Non-Small Cell, Lung Cancer with Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

132. The method of claim 131, wherein the patient has a myeloid cancer.

133. The method of claim 131, wherein the patient has AML or CML.

134. The method of any one of claim 101, wherein the patient has an inflammatory or autoimmune disease.

135. The method of claim 134, wherein the inflammatory or autoimmune disease is selected from the group consisting of: type-1 diabetes, autoimmune uveitis, autoimmune hepatitis, vitiligo, alopecia areata, rheumatoid arthritis, psoriasis, psoriatic arthritis, multiple sclerosis, systemic lupus, inflammatory bowel disease, Addison's disease, Graves' disease, Sjögren's syndrome, Hashimoto's thyroiditis, myasthenia gravis, autoimmune vasculitis, pernicious anemia, ulcerative colitis, bullous diseases, scleroderma, and celiac disease.

136. The method of claim 135, wherein the inflammatory or autoimmune disease is Crohn's disease, ulcerative colitis, celiac disease, type-1 diabetes, lupus, psoriasis, psoriatic arthritis, or rheumatoid arthritis.

137. The method of any one of claims 101-129 or 134-136, wherein the patient has a disease or disorder involving hyperactivity of NLPR3 inflammasome.

138. The method of any one of claims 101-129 or 134-137, wherein the patient has type 2 diabetes, neurodegenerative diseases, cardiovascular diseases or inflammatory bowel disease.

139. The method of any one of claims 101-129 or 134-137, wherein the patient has a disease or disorder involving increased IL-1β production by activated monocytes, macrophages or dendritic cells.

140. The method of any one of claims 101-129 or 134-137, wherein the patient has a disease or disorder involving increased IL-18 production by activated monocytes, macrophages or dendritic cells.

141. The method of any one of claims 101-129 or 134-137, wherein the patient has a disease or disorder involving increased mature caspase 1 production by activated monocytes, macrophages or dendritic cells.

142. The method of any one of claims 101-129, wherein the patient has an allergic or atopic disease.

143. The method of claim 142, wherein the allergic or atopic disease is selected from the group consisting of: asthma, atopic dermatitis, and rhinitis.

144. The method of any one of claims 101-129, wherein the patient has a food allergy.

145. The method of any one of claims 101-129, further comprising the step of organ transplantation to the patient, either prior to or subsequent to administration of the population of CD4⁺ T cells or the pharmaceutical composition.

146. The method of claim 145, wherein the polydonor CD4^{IL-10} cells or the pharmaceutical composition prevents or reduces severity of host rejection of the organ transplantation.

147. The method of any one of claims 101-129, further comprising the step of transplanting iPS cell-derived cells or tissues to the patient, either prior to or subsequent to administration of the population of CD4⁺ T cells or the pharmaceutical composition.

148. The method of claim 147, wherein polydonor CD4^{IL-10} cells or the pharmaceutical composition prevents or reduces severity of host rejection of the cell transplantation.

149. The method of any one of claims 101-129, further comprising the step of administering a recombinant AAV to the patient, either prior to or subsequent to administration of the polydonor CD4^{IL-10} cells or the pharmaceutical composition.

150. The method of claim 149, wherein the polydonor CD4^{IL-10} cells or the pharmaceutical composition reduces immune responses against the recombinant AAV.

151. The method of claim 150, further comprising administering an immunogenic therapeutic protein to the patient, either prior to or subsequent to administration of the CD4^{IL-10/CAR} cells, the population of CD4^{IL-10/CAR} cells, or the pharmaceutical composition.

152. The method of claim 151, wherein the CD4^{IL-10/CAR} cells, the population of CD4^{IL-10/CAR} cells, or the pharmaceutical composition reduces immune responses against the immunogenic therapeutic protein.

153. The method of claim 151 or 152, wherein the immunogenic therapeutic protein is selected from a therapeutic antibody, a factor VIII replacement, a cytokine, and a cytokine mutein.

154. The method of any one of claims 101-129, wherein the patient has an excessive immune response against viral or bacterial infection.

155. The method of claim 154, wherein the patient has a coronavirus infection.

156. The method of claim 149 or 155, wherein the patient has organ and/or tissue damage.

157. The method of any one of claims 101-156, further comprising the step of detecting the selection marker in a biological sample obtained from the patient,
thereby detecting presence or absence of polydonor CD4^{IL-10} T cells.

158. The method of claim 157, wherein the biological sample is a biopsy or blood from the patient.

159. A method of treating a patient with a malignancy, comprising:
administering an allo-HSCT to the patient, and
administering a therapeutically effective amount of polydonor CD4^{IL-10} cells.

160. The method of claim 159, wherein none of the donors of the CD4^{IL-10} cells in the polydonor CD4^{IL-10} cells is the donor of the HSCT.

161. A method of treating a hematological cancer, comprising:
administering to a hematological cancer patient an amount of polydonor CD4^{IL-10} cells sufficient induce anti-cancer effect,
wherein the polydonor CD4^{IL-10} cells comprise CD4⁺ T cells obtained from at least two different T cell donors and genetically modified by vector-mediated gene transfer of the coding sequence of human IL-10 under control of a constitutive or inducible promoter.

162. The method of claim 161, further comprising the step of administering allo HSCT to the patient prior to or subsequent to administration of the polydonor CD4^{IL-10} cells.

163. The method of claim 162, wherein the amount of polydonor CD4^{IL-10} cells is further sufficient to suppress or prevent graft versus host disease (GvHD) without suppressing graft versus leukemia (GvL) or graft versus tumor (GvT) efficacy of the allo HSCT.

164. The method of any one of claims 161-163, wherein the hematological cancer is a myeloid leukemia.

165. The method of any one of claims 161-162, wherein the polydonor CD4^{IL-10} cells target and kill cancer cells that express CD13.

166. The method of any one of claims 161-165, wherein the polydonor CD4^{IL-10} cells target and kill cancer cells that express HLA-class I.

167. The method of any one of claims 161-166, wherein the myeloid leukemia is acute myeloid leukemia (AML).

168. The method of any one of claims 161-167, wherein the allo-HSCT is obtained from a related or unrelated donor with respect to the recipient.

169. The method of any one of claims 161-168, wherein the polydonor CD4^{IL-10} cells are non-autologous to the recipient.

170. The method of any one of claims 161-168, wherein the polydonor CD4^{IL-10} cells are allogeneic to the recipient.

171. The method of any one of claims 161-168, wherein the polydonor CD4^{IL-10} cells are not anergized to host allo-antigens prior to administration to the host.

172. The method of any one of claims 161-168, wherein the polydonor CD4^{IL-10} cells are Trn-like cells.

173. The method of any one of claims 161-168, wherein the polydonor CD4^{IL-10} cells are polyclonal.

174. The method of any one of claims 161-168, wherein the polydonor CD4^{IL-10} cells are polyclonal and non-autologous to the recipient.

175. The method of any one of claims 161-168, wherein the polydonor CD4^{IL-10} cells are isolated from at least two donors prior to being genetically modified.

176. The method of claim 170, wherein none of the at least two donors is the same donor as the allo-HSCT donor.

177. The method of any one of claims 161-176, wherein the allo-HSCT is obtained from a matched or mismatched donor with respect to the recipient.

178. The method of any one of claims 161-177, wherein the polydonor CD4^{IL-10} cells target and kill cells that express CD54.

179. The method of any one of claims 161-178, wherein the polydonor CD4^{IL-10} cells target and kill cancer cells that express HLA-class I and CD54.

180. The method of any one of claims 161-179, wherein the polydonor CD4^{IL-10} cells target and kill cancer cells that express CD112.

181. The method of any one of claims 161-180, wherein the polydonor CD4^{IL-10} cells target and kill cancer cells that express CD58.

182. The method of any one of claims 161-181, wherein the polydonor CD4^{IL-10} cells target and kill cancer cells in the host.

183. A method of treating a hematological cancer by allogeneic hematopoietic stem cell transplant (allo-HSCT), comprising:
administering allo-HSCT to a subject;
administering to the subject an amount of polydonor CD4^{IL-10} cells sufficient to suppress or prevent graft-versus-host disease (GvHD) without suppressing graft-versus-leukemia (GvL) or graft-versus-tumor (GvT) efficacy of the allo-HSCT;
wherein the polydonor CD4^{IL-10} cells comprise CD4⁺ T cells obtained from at least two different T cell donors and genetically modified by vector-mediated gene transfer of the coding sequence of human IL-10 under control of a constitutive or inducible promoter;
wherein the polydonor CD4^{IL-10} cells are non-autologous to the subject and non-autologous to the allo-HSCT donor;
wherein the polydonor CD4^{IL-10} cells are not anergized to subject's allo-antigens prior to administration to the subject; and
wherein the polydonor CD4^{IL-10} cells are polyclonal and Tr1-like.

184. The method of claim 183, wherein the allo-HSCT is administered after administration of the polydonor CD4^{IL-10} cells.

185. The method of claim 183, wherein the allo-HSCT is administered before administration of the polydonor CD4^{IL-10} cells.

186. A method of treating a hematological cancer by allogeneic hematopoietic stem cell transplant (allo-HSCT), comprising:
administering allo-HSCT to a subject;
administering to the subject an amount of polydonor CD4^{IL-10} cells sufficient to suppress or prevent graft-versus-host disease (GvHD) without suppressing graft-versus-leukemia (GvL) or graft-versus-tumor (GvT) efficacy of the allo-HSCT,
wherein the polydonor CD4^{IL-10} cells comprise CD4⁺ T cells obtained from at least two different T cell donors and genetically modified by vector-mediated gene transfer of the coding sequence of human IL-10 under control of a constitutive or inducible promoter,
wherein the polydonor CD4^{IL-10} cells target and kill cancer cells in the subject,
wherein the polydonor CD4^{IL-10} cells are not anergized to subject's allo-antigens prior to administration to the subject; and
wherein the polydonor CD4^{IL-10} cells are non-autologous to the subject, and polyclonal, and are Tr1-like.

187. The method of claim 186, wherein the allo-HSCT is administered after administration of the polydonor CD4^{IL-10} cells.

188. The method of claim 186, wherein the allo-HSCT is administered before administration of the polydonor CD4^{IL-10} cells.
